(19) United States
(12) Patent Application Publication
(10) Pub. No.: US 2008/0233127 A1
(43) Pub. Date: Sep. 25, 2008

(54) IMIDAZOLOPYRIMIDINE ANALOGS AND THEIR USE AS PI3 KINASE AND MTOR INHIBITORS

(75) Inventors: Matthew Gregory Bursavich, Sandy, UT (US); Aranapakam Mudumbai Venkatesan, Rego Park, NY (US); Pawel Wojciech Nowak, Montvale, NJ (US); Sabrina Lombardi, New Haven, CT (US); Adam Matthew Gilbert, Congers, NY (US); Christoph Martin Denhardt, New York, NY (US); Osvaldo Dos Santos, Astoria, NY (US); Efren Guillermo Delos Santos, Nanuet, NY (US); Natasja Brooijmans, New York, NY (US); Semiramis Ayral-Kaloustian, Tarrytown, NY (US); Zecheng Chen, New City, NY (US); Jeroen Cunera Verheijen, Highland Mills, NY (US); Joshua Kaplan, Nyack, NY (US); Arie Zask, New York, NY (US)

Correspondence Address: WYETH;PATENT LAW GROUP, 5 GIRALDA FARMS, MADISON, NJ, 07940, US

(73) Assignee: Wyeth, Madison, NJ (US)

(21) Appl. No.: 12/044,500

(22) Filed: Mar. 7, 2008

Related U.S. Application Data
(60) Provisional application No. 60/919,300, filed on Mar. 21, 2007.

(51) Int. Cl. A61K 39/395; C07D 473/00; A61K 31/5377; A61P 35/04; A61P 3/00; A61P 1/00; A61P 29/00; A61P 19/00; A61K 31/52

(52) U.S. Cl. 424/145.1; 544/264; 544/122; 514/234.2; 514/263.1

(57) ABSTRACT

The present invention relates to Imidazolopyrimidine Analogs, methods of making Imidazolopyrimidine Analogs, compositions comprising an Imidazolopyrimidine Analog, and methods for treating or preventing a PI3K-related disorder comprising administering to a subject in need thereof an effective amount of an Imidazolopyrimidine Analog. The invention also relates to methods for treating or preventing mTOR-related disorders comprising administering to a subject in need thereof an effective amount of an Imidazolopyrimidine Analog.

### FIELD OF THE INVENTION

The invention relates to Imidazolopyrimidine Analogs compositions comprising an Imidazolopyrimidine Analog and methods for treating or preventing PI3K-related diseases comprising the administration of an effective amount of an Imidazolopyrimidine Analog. The invention also relates to methods for treating or preventing mTOR-related diseases comprising the administration of an effective amount of an Imidazolopyrimidine Analog.

### BACKGROUND OF THE INVENTION

Phosphatidylinositol (hereinafter abbreviated as “PI”) is one of phospholipids in cell membranes. In recent years it has become clear that PI plays an important role also in intracellular signal transduction. It is well recognized in the art that especially PI (4,5) bisphosphate (PI(4,5)P2) is degraded into diacylglycerol and inositol (1,4,5) triphosphate by phospholipase C to induce activation of protein kinase C and intracellular calcium mobilization, respectively [M. J. Berridge et al., Nature, 312, 315 (1984); Y. Nishizuka, Science, 225, 1365 (1984)].

In the late 1980s, phosphatidylinositol-3 kinase (“PI3K”) was found to be an enzyme that phosphorylates the 3-position of the inositol ring of phosphatidylinositol [D. Whitman et al., Nature, 332, 664 (1988)].

When PI3K was discovered, it was originally considered to be a single enzyme. Recently however, it was clarified that a plurality of subtypes are present in PI3K. Three major classes of PI3Ks have now been identified on the basis of their in vitro substrate specificity [B. Vanhaesebroeck, Trend in Biol. Sci., 22, 267(1997)].

Substrates for class I PI3Ks are PI, PI(4)P and PI(4,5)P2. In these substrates, PI(4,5)P2 is the most advantageous substrate in cells. Class I PI3Ks are further divided into two groups, class Ia and class Ib, in terms of their activation mechanism. Class Ia PI3Ks, which include PI3K p110α, p110β and p110δ subtypes, are activated in the tyrosine kinase system. Class Ib PI3K is a p110γ subtype activated by a G protein-coupled receptor.

PI and PI(4)P are known as substrates for class II PI3Ks but PI(4,5)P2 is not a substrate for the enzymes of this class. Class II PI3Ks include PI3K C2α, C2β and C2γ subtypes, which are characterized by containing C2 domains at the C terminus, implying that their activity will be regulated by calcium ions.

The substrate for class III PI3Ks is PI only. A mechanism for activation of the class III PI3Ks is not clarified yet. Because each subtype has its own mechanism for the regulating activity, it is considered that the respective subtypes will be activated depending on their respective stimuli specific to each of them.

In the PI3K subtypes, the class Ia subtype has been most extensively investigated to date. The three subtypes of class Ia are hetero dimers of a catalytic 110 kDa subunit and regulatory subunits of 85 kDa and 55 kDa. The regulatory subunits contain SH2 domains and bind to tyrosine residues phosphorylated by growth factor receptors with a tyrosine kinase activity or oncogene products, thereby inducing the PI3K activity of the p110 catalytic subunit. Thus, the class Ia subtypes are considered to be associated with cell proliferation and carcinogenesis. Furthermore, the class Ia PI3K subtypes bind to activated ras oncogene to express their enzyme activity. It has been confirmed that the activated ras oncogene is present in many cancers, suggesting a role of class Ia PI3Ks in carcinogenesis.

Mammalian Target of Rapamycin, mTOR, is a cell-signaling protein that regulates the response of tumor cells to nutrients and growth factors, as well as controlling tumor blood supply through effects on Vascular Endothelial Growth Factor, VEGF. Inhibitors of mTOR starve cancer cells and shrink tumors by inhibiting the effect of mTOR. All mTOR inhibitors bind to the mTOR kinase. This has at least two important effects. First, mTOR is a downstream mediator of the PI3K/Akt pathway. The PI3K/Akt pathway is thought to be over activated in numerous cancers and may account for the widespread response from various cancers to mTOR inhibitors. The over activation of the upstream pathway would normally cause mTOR kinase to be over activated as well. However, in the presence of mTOR inhibitors, this process is blocked. The blocking effect prevents mTOR from signaling to downstream pathways that control cell growth. The interruption of the cell growth cycle may account for the fact that inhibitors are more likely to cause disease stability than shrinkage. Over activation of the PI3K/Akt kinase is frequently associated with mutations in the PTEN gene, which is common in many cancers and may help predict what tumors will respond to mTOR inhibitors. The second major effect of mTOR inhibition is anti-angiogenesis, via the lowering of VEGF levels.

In lab tests certain chemotherapy agents were found to be more effective in the presence of mTOR inhibitors. Geoerger, B., et al., Cancer Research, 2001, 61, 1527-1532. Additional lab results have shown that some rhabdomyosarcoma cells die in the presence of mTOR inhibitors. The complete functions of the mTOR kinase and the effects of mTOR inhibition are not completely understood.

As explained above, PI3K inhibitors and mTOR inhibitors are expected to be novel types of medicaments useful against cell proliferation disorders, especially as carcinostatic agents. Thus, it would be advantageous to have new PI3K inhibitors and mTOR inhibitors as potential treatment regimens for PI3K- and mTOR-related diseases. The instant invention is directed to these and other important ends.

### SUMMARY OF THE INVENTION

In one aspect, the invention provides compounds of the Formula I:

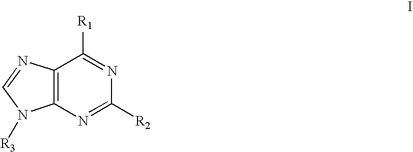

and pharmaceutically acceptable salts thereof, wherein: R₁, R₂ and R₃ are as defined below for compounds of Formula I.

In another aspect, the invention provides compounds of Formula Ia:

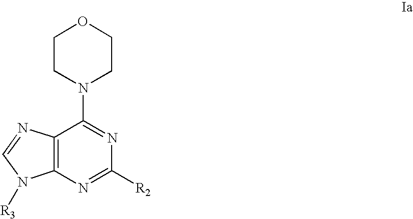

and pharmaceutically acceptable salts thereof, wherein R₂ and R₃ are as defined below for the compounds of Formula Ia.

In one aspect, the invention provides compounds of the Formula Ib

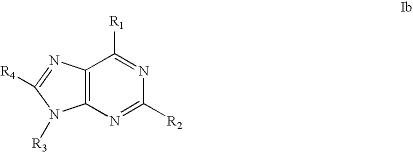

and pharmaceutically acceptable salts thereof, wherein: R₁, R₂, R₃, and R₄ are as defined below for compounds of Formula Ib

In one aspect, the invention provides compounds of the Formula Ic

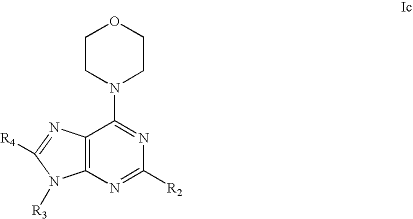

and pharmaceutically acceptable salts thereof, wherein: R₁, R₂, R₃, and R₄ are as defined below for compounds of Formula Ic

In another aspect, the invention provides compounds of Formula II:

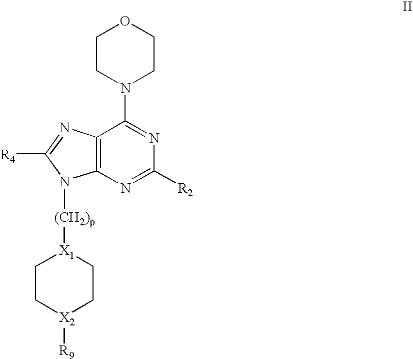

and pharmaceutically acceptable salts thereof; wherein R₂, R₄, R₉, X₁, X₂, and p are as defined below for the compounds of Formula II.

In another aspect, the invention provides compounds of Formula Ia:

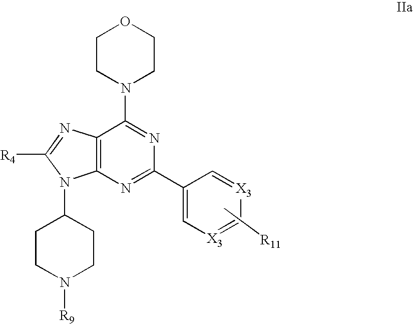

and pharmaceutically acceptable salts thereof, wherein R₄, R₉, R₁₁, and X₃ are as defined below for compounds of Formula IIa.

In another aspect, the invention provides compounds of Formula IIb:

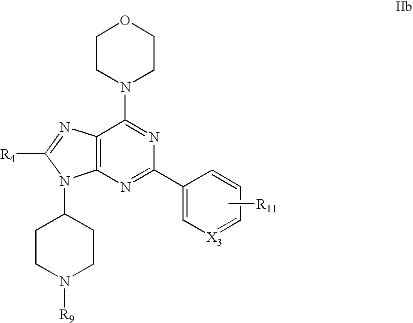

and pharmaceutically acceptable salts thereof, wherein R₄, R₉, R₁₁ and X₃ are as defined above for the compounds of Formula IIb.

### DETAILED DESCRIPTION OF THE INVENTION

In one aspect, the invention provides compounds of the Formula I:

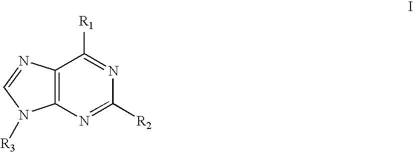

and pharmaceutically acceptable salts thereof,
wherein:
R₁ is N-morpholinyl, N-thiomorpholinyl, wherein the N-thiomorpholinyl sulfur atom may be substituted with one or two ═O, N-piperidinyl, or N-piperazinyl, wherein any one or more of the ring hydrogen atoms of the N-morpholinyl, N-thiomorpholinyl, N-piperidinyl, and N-piperazinyl can independently be substituted with C₁-C₃alkyl, C₁-C₃alkenyl, C₁-C₃alkynyl, C₁-C₃alkoxy, C₁-C₃ acyl, C₁-C₃acyloxy, -alkylamino, ═O, fluorine, or —CN;
R₂ is optionally substituted C₁-C₆ alkyl, optionally substituted C₂-C₁₀ alkenyl, provided that the substituent is not attached to a carbon of the double bond, optionally substituted C₂-C₁₀alkynyl, provided that the substituent is not attached to a carbon of the triple bond, optionally substituted aryl, optionally substituted heteroaryl, optionally substituted arylurea, optionally substituted arylcarbamate, optionally substituted —HC═CH-aryl, or optionally substituted —HC═CH-heteroaryl;
R₃ is hydrogen, optionally substituted C₁-C₆ alkyl, optionally substituted C₂-C₁₀ alkenyl, provided that the substituent is not attached to a carbon of the double bond, optionally substituted C₂-C₁₀alkynyl, provided that the substituent is not attached to a carbon of the triple bond, optionally substituted aryl, optionally substituted heteroaryl, alkylheterocycle, alkanol, alkylcarboxy, alkylamino-alkyloxy, C₁-C₆ perfluoro alkyl, —S(O)_{q}—C₁-C₆alkyl wherein the C₁-C₆alkyl of —S(O)_{q}—C₁-C₆alkyl can be optionally substituted, —S(O)_{q}-aryl wherein the aryl of —S(O)_{q}-aryl can be optionally substituted, optionally substituted C₃-C₈ carbocycle, 3- to 7-membered monocyclic heterocycle, nitrogen containing 3- to 7-membered monocyclic heterocycle, 7- to 10-membered bicyclic heterocycle, or nitrogen-containing 7- to 10-membered bicyclic heterocycle, wherein the nitrogen of the nitrogen containing 3- to 7-membered monocyclic heterocycle; and
q is 0, 1 or 2.

In another embodiment, R₁ is N-thiomorpholinyl.

In one embodiment, R₂ is optionally substituted aryl.

In one embodiment, R₂ is optionally substituted heteroaryl.

In another embodiment, R₂ is optionally substituted arylurea.

In another embodiment, R₂ is optionally substituted arylcarbamate.

In another embodiment, R₂ is —HC═CH-aryl.

In one embodiment, R₃ is hydrogen.

In one embodiment, R₃ is optionally substituted C₁-C₆ alkyl.

In one embodiment, R₃ is optionally substituted aryl.

In one embodiment, R₃ is optionally substituted heteroaryl.

In one embodiment, R₃ is —S(O)_{q}—C₁-C₆ alkyl.

In one embodiment, R₃ is —S(O)_{q}-aryl.

In one embodiment, R₃ is a 3- to 7-membered monocyclic heterocycle,

In one embodiment, R₃ is 7- to 10-membered bicyclic heterocycle.

In one embodiment, q is 0.

In one embodiment, q is 1.

In one embodiment, q is 2.

In another aspect, the invention provides compounds of Formula Ia:

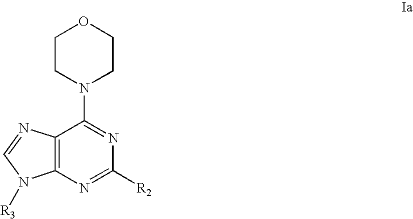

and pharmaceutically acceptable salts thereof,
wherein
R₂ is optionally substituted C₁-C₆ alkyl, optionally substituted C₂-C₁₀ alkenyl, provided that the substituent is not attached to a carbon of the double bond, optionally substituted C₂-C₁₀alkynyl, provided that the substituent is not attached to a carbon of the triple bond, optionally substituted aryl, optionally substituted heteroaryl, optionally substituted arylurea, optionally substituted arylcarbamate, optionally substituted —HC═CH-aryl, or optionally substituted —HC═CH-heteroaryl;
R₃ is hydrogen, optionally substituted C₁-C₆ alkyl, optionally substituted C₂-C₁₀ alkenyl, provided that the substituent is not attached to a carbon of the double bond, optionally substituted C₂-C₁₀alkynyl, provided that the substituent is not attached to a carbon of the triple bond, optionally substituted aryl, optionally substituted heteroaryl, alkylheterocycle, alkanol, alkylcarboxy, alkylamino-alkyloxy, C₁-C₆ perfluoro alkyl, —S(O)_{q}—C₁-C₆alkyl wherein the C₁-C₆alkyl of —S(O)_{q}—C₁-C₆alkyl can be optionally substituted, —S(O)_{q}-aryl wherein the aryl of —S(O)_{q}-aryl can be optionally substituted, optionally substituted C₃-C₈ carbocycle, 3- to 7-membered monocyclic heterocycle, nitrogen containing 3- to 7-membered monocyclic heterocycle, 7- to 10-membered bicyclic heterocycle, or nitrogen-containing 7- to 10-membered bicyclic heterocycle; and
q is 0, 1 or 2.

In one embodiment, R₁ is N-morpholinyl.

In one embodiment, R₂ is optionally substituted aryl.

In one embodiment, R₂ is optionally substituted heteroaryl.

In another embodiment, R₂ is optionally substituted arylurea.

In another embodiment, R₂ is optionally substituted arylcarbamate.

In another embodiment, R₂ is —HC═CH-aryl.

In one embodiment, R₃ is hydrogen.

In one embodiment, R₃ is optionally substituted C₁-C₆ alkyl.

In one embodiment, R₃ is optionally substituted aryl.

In one embodiment, R₃ is optionally substituted heteroaryl.

In one embodiment, R₃ is —S(O)_{q}—C₁-C₆ alkyl.

In one embodiment, R₃ is —S(O)_{q}-aryl.

In one embodiment, R₃ is a 3- to 7-membered monocyclic heterocycle.

In one embodiment, R₃ is 7- to 10-membered bicyclic heterocycle.

In one embodiment, q is 0.

In one embodiment, q is 1.

In one embodiment, q is 2.

In one aspect, the invention provides compounds of the Formula Ib:

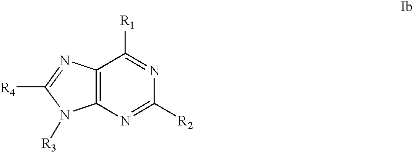

and pharmaceutically acceptable salts thereof, wherein:
R₁ is N-morpholinyl or N-thiomorpholinyl, wherein the N-thiomorpholinyl sulfur atom may be substituted with one or two ═O, wherein any one or more of the ring hydrogen atoms of the N-morpholinyl or N-thiomorpholinyl can independently be substituted with C₁-C₆alkyl, C₂-C₆alkenyl, C₂-C₆alkynyl, C₁-C₃alkoxy, C₁-C₃acyl, C₁-C₃alkylcarboxy, (C₁-C₆alkyl)amino, fluorine, or —CN;
where any two hydrogen atoms attached to the same carbon atom in R₁ can be replaced by an oxygen atom, where the oxygen atom taken together with the carbon to which it is attached, forms a carbonyl (C═O);
R₂ is
   (a) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
   (b) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
   (c) C₆-C₁₄aryl optionally substituted with from 1 to 3 substituents independently selected from:
      (i) halogen,
      (ii) C₁-C₆alkyl,
      (iii) C₁-C₆alkoxy, optionally substituted with C₁-C₆alkoxy,
      (iv) C₁-C₆hydroxylalkyl,
      (v) C₂-C₆alkenyl,
      (vi) C₂-C₆alkynyl,
      (vii) C₃-C₈carbocycle,
      (viii) C₆-C₁₄aryl,
      (ix) C₁-C₉heteroaryl,
      (x) C₁-C₆perfluoroalkyl-,
      (xi) hydroxyl,
      (xii) NR₁₂R₁₂,
      (xiii) NO₂,
      (xiv) CN,
      (xv) CO₂H,
      (xvi) CHO,
      (xvii) C₆-C₁₄aryl-O—,
      (xviii) (C₆-C₁₄aryl)alkyl-O—, optionally substituted with from 1 to 3 C₁-C₆alkoxy,
      (xix) —C(O)NR₁₂R₁₂,
      (xx) NHC(O)R₁₃,
      (xxi) —NHC(O)NR₁₂R₁₂,
      (xxii) —NHC(O)OR₁₃,
      (xxiii) —NH(SO₂)—(C₁-C₆alkyl),
      (xxiv) and —(SO₂)—(C₁-C₆alkyl);
where any two hydrogen atoms on adjacent carbon atoms of the C₆-C₁₄aryl can be replaced by an alkylenedioxy group so that the alkylenedioxy group, when taken together with the two carbon atoms to which it is attached, form a 5- to 7-membered heterocycle containing two oxygen atoms;
   (d) or C₁-C₉heteroaryl optionally substituted with from 1 to 3 substituents independently selected from:
      (i) halogen,
      (ii) C₁-C₆alkyl,
      (iii) C₁-C₆alkoxy, optionally substituted with C₁-C₆alkoxy,
      (iv) C₁-C₆hydroxylalkyl,
      (v) C₂-C₆alkenyl,
      (vi) C₂-C₆alkynyl,
      (vii) C₃-C₈carbocycle,
      (viii) C₆-C₁₄aryl,
      (ix) C₁-C₉heteroaryl,
      (x) C₁-C₆perfluoroalkyl-,
      (xi) hydroxyl,
      (xii) NR₁₂R₁₂,
      (xiii) NO₂,
      (xiv) CN,
      (xv) CO₂H,
      (xvi) CHO,
      (xvii) C₆-C₁₄aryl-O—,
      (xviii) (C₆-C₁₄aryl)alkyl-O—, optionally substituted with from 1 to 3 C₁-C₆alkoxy,
      (xix) —C(O)NR₁₂R₁₂,
      (xx) NHC(O)R₁₃,
      (xxi) —NHC(O)NR₁₂R₁₂,
      (xxii) —NHC(O)OR₁₃,
      (xxiii) —NH(SO₂)—(C₁-C₆alkyl),
      (xxiv) and —(SO₂)—(C₁-C₆alkyl);
where any two hydrogen atoms on adjacent carbon atoms of the C₁-C₉heteroaryl can be replaced by an alkylenedioxy group so that the alkylenedioxy group, when taken together with the two carbon atoms to which it is attached, form a 5- to 7-membered heterocycle containing two oxygen atoms;
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl;
R₃ is:
   (a) hydrogen;
   (b) C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (c) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
   (d) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
   (e) C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (f) C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (g) C₃-C₈carbocycle, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (h) heterocyclyl(C₁-C₆alkyl) optionally substituted with (C₆-C₁₄aryl)alkyl;
   (i) 3- to 7-membered monocyclic heterocycle optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl;
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (j) nitrogen containing 3- to 7-membered monocyclic heterocycle optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (k) 7- to 10-membered bicyclic heterocycle optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
      H) C₆-C₁₄aryl,
         I) and C₁-C₉heteroaryl,
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (l) or nitrogen-containing 7- to 10-membered bicyclic heterocycle, wherein the nitrogen of the nitrogen containing 3- to 7-membered monocyclic heterocycle is optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl;
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (m) C₁-C₆hydroxylalkyl;
   (n) C₁-C₆alkylcarboxy;
   (o) C₁-C₆perfluoroalkyl;
   (p) —S(O)_{q}—C₁-C₆alkyl;
   (q) or —S(O)_{q}—C₆-C₄aryl;
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
q is 0, 1 or 2;
with the proviso that R₃ and R₄ cannot simultaneously be unsubstituted C₁-C₆alkyl;
and that 3-[6-(4-morpholinyl)-9H-purin-2-yl]-phenol is excluded.

In another embodiment, R₁ is N-morpholinyl.

In one embodiment, R₂ is C₆-C₁₄aryl optionally substituted with from 1 to 3 substituents as specified in Formula Ib.

In one embodiment, R₂ is C₁-C₉heteroaryl optionally substituted with from 1 to 3 substituents as specified in Formula Ib.

In another embodiment, R₂ is C₆-C₁₄aryl substituted with from 1 to 3 —NHC(O)NR₁₂R₁₂.

In another embodiment, R₂ is C₆-C₁₄aryl substituted from 1 to 3 NHC(O)R₁₃.

In one embodiment, R₃ is hydrogen.

In one embodiment, R₃ is C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

In one embodiment, R₃ is C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

In one embodiment, R₃ is C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

In one embodiment, R₃ is —S(O)_{q}—C₁-C₆ alkyl.

In one embodiment, R₃ is —S(O)_{q}-aryl.

In one embodiment, R₃ is a 3- to 7-membered monocyclic heterocycle, optionally substituted with from 1 to 3 substituents as specified in Formula Ib.

In one embodiment, R₃ is 7- to 10-membered bicyclic heterocycle optionally substituted with from 1 to 3 substituents as specified in Formula Ib.

In one embodiment, q is 0.

In one embodiment, q is 1.

In one embodiment, q is 2.

In another aspect, the invention provides compounds of Formula Ic:

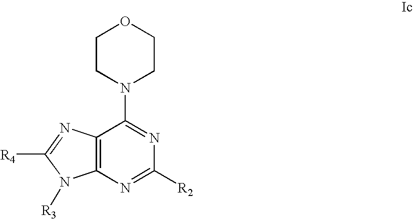

and pharmaceutically acceptable salts thereof, wherein
R₂ is
   (a) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
   (b) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
   (c) C₆-C₁₄aryl optionally substituted with from 1 to 3 substituents independently selected from:
      (i) halogen,
      (ii) C₁-C₆alkyl,
      (iii) C₁-C₆alkoxy, optionally substituted with C₁-C₆alkoxy,
      (iv) C₁-C₆hydroxylalkyl,
      (v) C₂-C₆alkenyl,
      (vi) C₂-C₆alkynyl,
      (vii) C₃-C₈carbocycle,
      (viii) C₆-C₁₄aryl,
      (ix) C₁-C₉heteroaryl,
      (x) C₁-C₆perfluoroalkyl-,
      (xi) hydroxyl,
      (xii) NR₁₂R₁₂,
      (xiii) NO₂,
      (xiv) CN,
      (xv) CO₂H,
      (xvi) CHO,
      (xvii) C₆-C₁₄aryl-O—,
      (xviii) (C₆-C₁₄aryl)alkyl-O—, optionally substituted with from 1 to 3 C₁-C₆alkoxy,
      (xix) —C(O)NR₁₂R₁₂,
      (xx) NHC(O)R₁₃,
      (xxi) —NHC(O)NR₁₂R₁₂,
      (xxii) —NHC(O)OR₁₃,
      (xxiii) —NH(SO₂)—(C₁-C₆alkyl),
      (xxiv) and —(SO₂)—(C₁-C₆alkyl);
where any two hydrogen atoms on adjacent carbon atoms of the C₆-C₁₄aryl can be replaced by an alkylenedioxy group so that the alkylenedioxy group, when taken together with the two carbon atoms to which it is attached, form a 5- to 7-membered heterocycle containing two oxygen atoms;
   (d) or C₁-C₉heteroaryl optionally substituted with from 1 to 3 substituents independently selected from:
      (i) halogen,
      (ii) C₁-C₆alkyl,
      (iii) C₁-C₆alkoxy, optionally substituted with C₁-C₆alkoxy,
      (iv) C₁-C₆hydroxylalkyl,
      (v) C₂-C₆alkenyl,
      (vi) C₂-C₆alkynyl,
      (vii) C₃-C₈carbocycle,
      (viii) C₆-C₁₄aryl,
      (ix) C₁-C₉heteroaryl,
      (x) C₁-C₆perfluoroalkyl-,
      (xi) hydroxyl,
      (xii) NR₁₂R₁₂,
      (xiii) NO₂,
      (xiv) CN,
      (xv) CO₂H,
      (xvi) CHO,
      (xvii) C₆-C₁₄aryl-O—,
      (xviii) (C₆-C₁₄aryl)alkyl-O—, optionally substituted with from 1 to 3 C₁-C₆alkoxy,
      (xix) —C(O)NR₁₂R₁₂,
      (xx) NHC(O)R₁₃,
      (xxi) —NHC(O)NR₁₂R₁₂,
      (xxii) —NHC(O)OR₁₃,
      (xxiii) —NH(SO₂)—(C₁-C₆alkyl),
      (xxiv) and —(SO₂)—(C₁-C₆alkyl);
where any two hydrogen atoms on adjacent carbon atoms of the C₁-C₉heteroaryl can be replaced by an alkylenedioxy group so that the alkylenedioxy group, when taken together with the two carbon atoms to which it is attached, form a 5- to 7-membered heterocycle containing two oxygen atoms;
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl;
R₃ is:
   (a) hydrogen;
   (b) C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (c) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
   (d) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
   (e) C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (f) C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (g) C₃-C₈carbocycle, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
   (h) heterocyclyl(C₁-C₆alkyl) optionally substituted with (C₆-C₁₄aryl)alkyl;
   (i) 3- to 7-membered monocyclic heterocycle optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl;
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (j) nitrogen containing 3- to 7-membered monocyclic heterocycle optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (k) 7- to 10-membered bicyclic heterocycle optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
      H) C₆-C₁₄aryl,
         I) and C₁-C₉heteroaryl,
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (l) or nitrogen-containing 7- to 10-membered bicyclic heterocycle, wherein the nitrogen of the nitrogen containing 3- to 7-membered monocyclic heterocycle is optionally substituted with one or more substituent independently selected from:
      (i) C₁-C₆alkyl,
      (ii) (C₁-C₆alkoxy)carbonyl,
      (iii) C₁-C₈acyl,
      (iv) (C₆-C₁₄aryl)alkyl, wherein the ring portion of the (C₆-C₁₄aryl)alkyl group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl;
      (v) heteroaryl(C₁-C₆alkyl), wherein the ring portion of the heteroaryl(C₁-C₆alkyl) group is optionally substituted by 1 to 3 substituents independently selected from:
         A) halogen,
         B) C₁-C₆alkyl,
         C) NH₂,
         D) (C₁-C₆alkyl)amino,
         E) di(C₁-C₆alkyl)amino,
         F) C₁-C₆alkoxy, wherein the C₁-C₆alkoxy is optionally substituted with NH₂, (C₁-C₆alkyl)amino, or di(C₁-C₆alkyl)amino,
         G) C₁-C₉heterocycle,
         H) C₆-C₁₄aryl,
         I) and C₁-C₉heteroaryl,
      (vi) —C(O)OH,
      (vii) halogen,
      (viii) —NH₂,
      (ix) —NH(C₁-C₆alkyl),
      (x) —N(C₁-C₆alkyl)(C₁-C₆alkyl),
      (xi) —N(C₁-C₃alkyl)C(O)(C₁-C₆alkyl),
      (xii) —NHC(O)(C₁-C₆alkyl),
      (xiii) —NHC(O)H,
      (xiv) —C(O)NH₂,
      (xv) —C(O)NH(C₁-C₆alkyl),
      (xvi) —C(O)N(C₁-C₆alkyl)(C₁-C₆alkyl),
      (xvii) —CN,
      (xviii) —OH,
      (xix) —O(C₁-C₆alkyl),
      (xx) C₆-C₁₄aryl,
      (xxi) C₁-C₉heteroaryl,
      (xxii) and C₃-C₈carbocycle;
   (m) C₁-C₆hydroxylalkyl;
   (n) C₁-C₆alkylcarboxy;
   (o) C₁-C₆perfluoroalkyl;
   (p) —S(O)_{q}—C₁-C₆alkyl;
   (q) or —S(O)_{q}—C₆-C₄aryl;
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
q is 0, 1 or 2;
with the proviso that R₃ and R₄ cannot simultaneously be unsubstituted C₁-C₆alkyl;
and that 3-[6-(4-morpholinyl)-9H-purin-2-yl]-phenol is excluded.

In another embodiment, R₁ is N-morpholinyl.

In one embodiment, R₂ is C₆-C₁₄aryl optionally substituted with from 1 to 3 substituents as specified in Formula Ic.

In one embodiment, R₂ is C₁-C₉heteroaryl optionally substituted with from 1 to 3 substituents as specified in Formula Ic.

In another embodiment, R₂ is C₆-C₁₄aryl substituted with from 1 to 3 —NHC(O)NR₁₂R₁₂.

In another embodiment, R₂ is C₆-C₁₄aryl substituted from 1 to 3 NHC(O)R₁₃.

In one embodiment, R₃ is hydrogen.

In one embodiment, R₃ is C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

In one embodiment, R₃ is C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

In one embodiment, R₃ is C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

In one embodiment, R₃ is —S(O)_{q}—C₁-C₆ alkyl.

In one embodiment, R₃ is —S(O)_{q}-aryl.

In one embodiment, R₃ is a 3- to 7-membered monocyclic heterocycle, optionally substituted with from 1 to 3 substituents as specified in Formula Ic.

In one embodiment, R₃ is 7- to 10-membered bicyclic heterocycle optionally substituted with from 1 to 3 substituents as specified in Formula Ic.

In one embodiment, q is 0.

In one embodiment, q is 1.

In one embodiment, q is 2.

In another aspect, the invention provides compounds of Formula II:

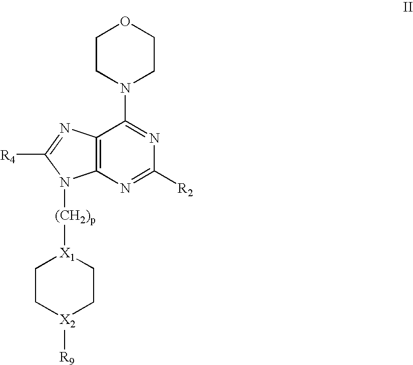

and pharmaceutically acceptable salts thereof; wherein
X₁ is —C(H)— or —N—;
X₂ is —C(H), —N—, —O—, or —S(O)ₙ—, provided that when X₂ is —O— or —S(O)ₙ—, R₉ is absent;
n is 0, 1, or 2;
p is 0, 1, 2, 3, 4, or 5, provided that when p is 0 a bond exists between the —N— and X₁ and X₁ cannot be —N—;
R₂ is:
   (a) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
   (b) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
   (c) C₆-C₁₄aryl optionally substituted with from 1 to 3 substituents independently selected from:
      (i) halogen,
      (ii) C₁-C₆alkyl,
      (iii) C₁-C₆alkoxy, optionally substituted with C₁-C₆alkoxy,
      (iv) C₁-C₆hydroxylalkyl,
      (v) C₂-C₆alkenyl,
      (vi) C₂-C₆alkynyl,
      (vii) C₃-C₈carbocycle,
      (viii) C₆-C₁₄aryl,
      (ix) C₁-C₉heteroaryl,
      (x) C₁-C₆perfluoroalkyl-,
      (xi) hydroxyl,
      (xii) NR₁₂R₁₂,
      (xiii) NO₂,
      (xiv) CN,
      (xv) CO₂H,
      (xvi) CHO,
      (xvii) C₆-C₁₄aryl-O—,
      (xviii) (C₆-C₁₄aryl)alkyl-O—, optionally substituted with from 1 to 3 C₁-C₆alkoxy,
      (xix) —C(O)NR₁₂R₁₂,
      (xx) NHC(O)R₁₃,
      (xxi) —NHC(O)NR₁₂R₁₂,
      (xxii) —NHC(O)OR₁₃,
      (xxiii) —NH(SO₂)—(C₁-C₆alkyl),
      (xxiv) and —(SO₂)—(C₁-C₆alkyl);
where any two hydrogen atoms on adjacent carbon atoms of the C₆-C₁₄aryl can be replaced by an alkylenedioxy group so that the alkylenedioxy group, when taken together with the two carbon atoms to which it is attached, form a 5- to 7-membered heterocycle containing two oxygen atoms;
   (d) or C₁-C₉heteroaryl optionally substituted with from 1 to 3 substituents independently selected from:
      (i) halogen,
      (ii) C₁-C₆alkyl,
      (iii) C₁-C₆alkoxy, optionally substituted with C₁-C₆alkoxy,
      (iv) C₁-C₆hydroxylalkyl,
      (v) C₂-C₆alkenyl,
      (vi) C₂-C₆alkynyl,
      (vii) C₃-C₈carbocycle,
      (viii) C₆-C₁₄aryl,
      (ix) C₁-C₉heteroaryl,
      (x) C₁-C₆perfluoroalkyl-,
      (xi) hydroxyl,
      (xii) NR₁₂R₁₂,
      (xiii) NO₂,
      (xiv) CN,
      (xv) CO₂H,
      (xvi) CHO,
      (xvii) C₆-C₁₄aryl-O—,
      (xviii) (C₆-C₁₄aryl)alkyl-O—, optionally substituted with from 1 to 3 C₁-C₆alkoxy,
      (xix) —C(O)NR₁₂R₁₂,
      (xx) NHC(O)R₁₃,
      (xxi) —NHC(O)NR₁₂R₁₂,
      (xxii) —NHC(O)OR₁₃,
      (xxiii) —NH(SO₂)—(C₁-C₆alkyl),
      (xxiv) and —(SO₂)—(C₁-C₆alkyl);
where any two hydrogen atoms on adjacent carbon atoms of the C₁-C₉heteroaryl can be replaced by an alkylenedioxy group so that the alkylenedioxy group, when taken together with the two carbon atoms to which it is attached, form a 5- to 7-membered heterocycle containing two oxygen atoms;
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl;
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
R₉ is:
   (a) hydrogen;
   (b) C₁-C₆alkyl;
   (c) (C₁-C₆alkoxy)carbonyl;
   (d) C₁-C₈acyl;
   (e) (C₆-C₁₄aryl)alkyl, wherein the ring portion of the (C₆-C₁₄aryl)alkyl group is optionally substituted by 1 to 3 substituents independently selected from:
      (i) halogen,
      (ii) C₁-C₆alkyl,
      (iii) NH₂,
      (iv) (C₁-C₆alkyl)amino,
      (v) di(C₁-C₆alkyl)amino,
      (vi) C₁-C₆alkoxy, wherein the C₁-C₆alkoxy is optionally substituted with NH₂, (C₁-C₆alkyl)amino, or di(C₁-C₆alkyl)amino,
      (vii) C₁-C₉heterocycle,
      (viii) C₆-C₁₄aryl,
      (ix) and C₁-C₉heteroaryl;
   (f) heteroaryl(C₁-C₆alkyl), wherein the ring portion of the heteroaryl(C₁-C₆alkyl) group is optionally substituted by 1 to 3 substituents independently selected from:
      (i) halogen,
      (ii) C₁-C₆alkyl,
      (iii) NH₂,
      (iv) (C₁-C₆alkyl)amino,
      (v) di(C₁-C₆alkyl)amino,
      (vi) C₁-C₆alkoxy, wherein the C₁-C₆alkoxy is optionally substituted with NH₂, (C₁-C₆alkyl)amino, or di(C₁-C₆alkyl)amino,
      (vii) C₁-C₉heterocycle,
      (viii) C₆-C₁₄aryl,
      (ix) and C₁-C₉heteroaryl;
   (g) —C(O)OH;
   (h) halogen;
   (i) —NH₂;
   (j) —NH(C₁-C₆alkyl);
   (k) —N(C₁-C₆alkyl)(C₁-C₆alkyl);
   (l) —N(C₁-C₃alkyl)C(O)(C₁-C₆alkyl);
   (m) —NHC(O)(C₁-C₆alkyl);
   (n) —NHC(O)H;
   (o) —C(O)NH₂;
   (p) —C(O)NH(C₁-C₆alkyl);
   (q) —C(O)N(C₁-C₆alkyl)(C₁-C₆alkyl);
   (r) —CN;
   (s) —OH;
   (t) —O(C₁-C₆alkyl);
   (u) C₆-C₁₄aryl,
   (v) C₁-C₉heteroaryl,
   (w) or C₃-C₈carbocycle.

In one embodiment, R₂ is C₆-C₁₄aryl optionally substituted with from 1 to 3 substituents as specified in Formula II.

In one embodiment, R₂ is C₁-C₉heteroaryl optionally substituted with from 1 to 3 substituents as specified in Formula II.

In another embodiment, R₂ is C₆-C₁₄aryl substituted with from 1 to 3 —NHC(O)NR₁₂R₁₂.

In another embodiment, R₂ is C₆-C₁₄aryl substituted from 1 to 3 NHC(O)R₁₃.

In one embodiment, R₄ is hydrogen.

In one embodiment, X₁ is —N—.

In one embodiment, X₁ is —C(H)—.

In some embodiments, X₁ is —C(H)— and X₂ is —N—.

In one embodiment, X₂ is —O— and R₉ is absent.

In some embodiments, p is 0.

In one embodiment, p is 1.

In some embodiments, R₉ is:
(a) C₁-C₆alkyl;
(b) (C₁-C₆alkoxy)carbonyl;
(c) C₁-C₈acyl;
(d) (C₆-C₁₄aryl)alkyl, wherein the ring portion of the (C₆-C₁₄aryl)alkyl group is optionally substituted by 1 to 3 substituents independently selected from:
   (i) halogen,
   (ii) C₁-C₆alkyl,
   (iii) NH₂,
   (iv) (C₁-C₆alkyl)amino,
   (v) di(C₁-C₆alkyl)amino,
   (vi) C₁-C₆alkoxy, wherein the C₁-C₆alkoxy is optionally substituted with NH₂, (C₁-C₆alkyl)amino, or di(C₁-C₆alkyl)amino,
   (vii) C₁-C₉heterocycle,
   (viii) C₆-C₁₄aryl,
   (ix) and C₁-C₉heteroaryl;
(e) heteroaryl(C₁-C₆alkyl), wherein the ring portion of the heteroaryl(C₁-C₆alkyl) group is optionally substituted by 1 to 3 substituents independently selected from:
   (i) halogen,
   (ii) C₁-C₆alkyl,
   (iii) NH₂,
   (iv) (C₁-C₆alkyl)amino,
   (v) di(C₁-C₆alkyl)amino,
   (vi) C₁-C₆alkoxy, wherein the C₁-C₆alkoxy is optionally substituted with NH₂, (C₁-C₆alkyl)amino, or di(C₁-C₆alkyl)amino,
   (vii) C₁-C₉heterocycle,
   (viii) C₆-C₁₄aryl,
   (ix) and C₁-C₉heteroaryl;
(f) —C(O)OH;
(g) halogen;
(h) —NH₂;
(i) —NH(C₁-C₆alkyl);
(j) —N(C₁-C₆alkyl)(C₁-C₆alkyl);
(k) —N(C₁-C₃alkyl)C(O)(C₁-C₆alkyl);
(l) —NHC(O)(C₁-C₆alkyl);
(m) —NHC(O)H;
(n) —C(O)NH₂;
(o) —C(O)NH(C₁-C₆alkyl);
(p) —C(O)N(C₁-C₆alkyl)(C₁-C₆alkyl);
(q) —CN;
(r) —OH;
(s) —O(C₁-C₆alkyl);
(t) C₆-C₁₄aryl,
(u) C₁-C₉heteroaryl,
(v) or C₃-C₈carbocycle.

In another aspect, the invention provides compounds of Formula IIa:

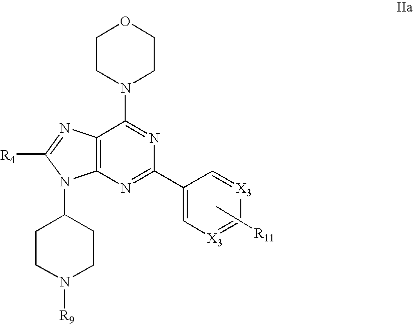

and pharmaceutically acceptable salts thereof, wherein
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
R₉ is:
   (a) hydrogen;
   (b) C₁-C₆alkyl;
   (c) (C₁-C₆alkoxy)carbonyl;
   (d) C₁-C₈acyl;
   (e) (C₆-C₁₄aryl)alkyl, wherein the ring portion of the (C₆-C₁₄aryl)alkyl group is optionally substituted by 1 to 3 substituents independently selected from:
      (i) halogen,
      (ii) C₁-C₆alkyl,
      (iii) NH₂,
      (iv) (C₁-C₆alkyl)amino,
      (v) di(C₁-C₆alkyl)amino,
      (vi) C₁-C₆alkoxy, wherein the C₁-C₆alkoxy is optionally substituted with NH₂, (C₁-C₆alkyl)amino, or di(C₁-C₆alkyl)amino,
      (vii) C₁-C₉heterocycle,
      (viii) C₆-C₁₄aryl,
      (ix) and C₁-C₉heteroaryl;
(f) heteroaryl(C₁-C₆alkyl), wherein the ring portion of the heteroaryl(C₁-C₆alkyl) group is optionally substituted by 1 to 3 substituents independently selected from:
   (i) halogen,
   (ii) C₁-C₆alkyl,
   (iii) NH₂,
   (iv) (C₁-C₆alkyl)amino,
   (v) di(C₁-C₆alkyl)amino,
   (vi) C₁-C₆alkoxy, wherein the C₁-C₆alkoxy is optionally substituted with NH₂, (C₁-C₆alkyl)amino, or di(C₁-C₆alkyl)amino,
   (vii) C₁-C₉heterocycle,
   (viii) C₆-C₁₄aryl,
   (ix) and C₁-C₉heteroaryl;
   (g) —C(O)OH;
   (h) halogen;
   (i) —NH₂;
   (j) —NH(C₁-C₆alkyl);
   (k) —N(C₁-C₆alkyl)(C₁-C₆alkyl);
   (l) —N(C₁-C₃alkyl)C(O)(C₁-C₆alkyl);
   (m) —NHC(O)(C₁-C₆alkyl);
   (n) —NHC(O)H;
   (o) —C(O)NH₂;
   (p) —C(O)NH(C₁-C₆alkyl);
   (q) —C(O)N(C₁-C₆alkyl)(C₁-C₆alkyl);
   (r) —CN;
   (s) —OH;
   (t) —O(C₁-C₆alkyl);
   (u) C₆-C₁₄aryl,
   (v) C₁-C₉heteroaryl,
   (w) or C₃-C₈carbocycle;
each X₃ is independently —N—, —C(H)—, —C((CH₂)_{w}—OH)—, or —C(C(O)H)—;
w is 0, 1, 2, 3, 4, or 5;
R₁₁ is
   (a) halogen;
   (b) C₁-C₆alkyl;
   (c) C₁-C₆alkoxy; optionally substituted with C₁-C₆alkoxy;
   (d) C₁-C₆hydroxylalkyl;
   (e) C₂-C₆alkenyl;
   (f) C₂-C₆alkynyl;
   (g) C₃-C₈carbocycle;
   (h) C₆-C₁₄aryl;
   (i) C₁-C₉heteroaryl;
   (j) C₁-C₆perfluoroalkyl-;
   (k) hydroxyl;
   (l) NR₁₂R₁₂;
   (m) NO₂;
   (n) CN;
   (o) CO₂H;
   (p) CHO;
   (q) C₆-C₁₄aryl-O—;
   (r) (C₆-C₁₄aryl)alkyl-O—; optionally substituted with from 1 to 3 C₁-C₆alkoxy;
   (s) —C(O)NR₁₂R₁₂;
   (t) NHC(O)R₁₃;
   (u) —NHC(O)NR₁₂R₁₂;
   (v) —NHC(O)OR₁₃;
   (w) —NH(SO₂)—(C₁-C₆alkyl);
   (x) or —(SO₂)—(C₁-C₆alkyl);
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl.

In one embodiment, R₄ is hydrogen.

In some embodiments, R₉ is C₁-C₆alkyl.

In some embodiments, R₉ is (C₁-C₆alkoxy)carbonyl.

In some embodiments, R₉ is C₁-C₈acyl.

In some embodiments, R₉ is (C₆-C₁₄aryl)alkyl, wherein the ring portion of the (C₆-C₁₄aryl)alkyl group is optionally substituted by 1 to 3 substituents as specified in Formula IIa.

In some embodiments, R₉ is heteroaryl(C₁-C₆alkyl), wherein the ring portion of the heteroaryl(C₁-C₆alkyl) group is optionally substituted by 1 to 3 substituents as specified in Formula IIa.

In some embodiments one X₃ is —N—.

In some embodiments, each X₃ is —N—.

In some embodiments, one X₃ is —C(H)—.

In some embodiments, X₃ is —C(C(O)H)—.

In some embodiments, R₁₁ is hydrogen.

In some embodiments, R₁₁ is hydroxyl.

In some embodiments, R₁₁ is —NR₁₂R₁₂.

In some embodiments, R₁₁ is —NHC(O)NR₁₂R₁₂.

In some embodiments, R₁₁ is —NHC(O)OR₁₃.

In some embodiments, R₁₂ is hydrogen.

In some embodiments, R₁₂ is C₁-C₆alkyl.

In some embodiments, R₁₂ is C₆-C₁₄aryl.

In some embodiments, R₁₃ is C₁-C₆alkyl.

In another aspect, the invention provides compounds of Formula IIb:

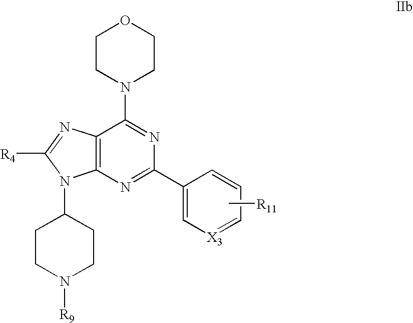

and pharmaceutically acceptable salts thereof, wherein
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
R₉ is:
   (a) hydrogen;
   (b) C₁-C₆alkyl;
   (c) (C₁-C₆alkoxy)carbonyl;
   (d) C₁-C₈acyl;
   (e) (C₆-C₁₄aryl)alkyl, wherein the ring portion of the (C₆-C₁₄aryl)alkyl group is optionally substituted by 1 to 3 substituents independently selected from:
      (i) halogen,
      (ii) C₁-C₆alkyl,
      (iii) NH₂,
      (iv) (C₁-C₆alkyl)amino,
      (v) di(C₁-C₆alkyl)amino,
      (vi) C₁-C₆alkoxy, wherein the C₁-C₆alkoxy is optionally substituted with NH₂, (C₁-C₆alkyl)amino, or di(C₁-C₆alkyl)amino,
      (vii) C₁-C₉heterocycle,
      (viii) C₆-C₁₄aryl,
      (ix) and C₁-C₉heteroaryl;
   (f) heteroaryl(C₁-C₆alkyl), wherein the ring portion of the heteroaryl(C₁-C₆alkyl) group is optionally substituted by 1 to 3 substituents independently selected from:
      (i) halogen,
      (ii) C₁-C₆alkyl,
      (iii) NH₂,
      (iv) (C₁-C₆alkyl)amino,
      (v) di(C₁-C₆alkyl)amino,
      (vi) C₁-C₆alkoxy, wherein the C₁-C₆alkoxy is optionally substituted with NH₂, (C₁-C₆alkyl)amino, or di(C₁-C₆alkyl)amino,
      (vii) C₁-C₉heterocycle,
      (viii) C₆-C₁₄aryl,
      (ix) and C₁-C₉heteroaryl;
   (g) —C(O)OH;
   (h) halogen;
   (i) —NH₂;
   (j) —NH(C₁-C₆alkyl);
   (k) —N(C₁-C₆alkyl)(C₁-C₆alkyl);
   (l) —N(C₁-C₃alkyl)C(O)(C₁-C₆alkyl);
   (m) —NHC(O)(C₁-C₆alkyl);
   (n) —NHC(O)H;
   (o) —C(O)NH₂;
   (p) —C(O)NH(C₁-C₆alkyl);
   (q) —C(O)N(C₁-C₆alkyl)(C₁-C₆alkyl);
   (r) —CN;
   (s) —OH;
   (t) —O(C₁-C₆alkyl);
   (u) C₆-C₁₄aryl,
   (v) C₁-C₉heteroaryl,
   (w) or C₃-C₈carbocycle;
X₃ is —N—, or —C(H)—;
R₁₁ is:
   (a) halogen,
   (b) C₁-C₆alkyl,
   (c) C₁-C₆alkoxy, optionally substituted with C₁-C₆alkoxy,
   (d) C₁-C₆hydroxylalkyl,
   (e) C₂-C₆alkenyl,
   (f) C₂-C₆alkynyl,
   (g) C₃-C₈carbocycle,
   (h) C₆-C₁₄aryl,
   (i) C₁-C₉heteroaryl,
   (j) C₁-C₆perfluoroalkyl-,
   (k) hydroxyl,
   (l) NR₁₂R₁₂,
   (m) NO₂,
   (n) CN,
   (o) CO₂H,
   (p) CHO,
   (q) C₆-C₁₄aryl-O—,
   (r) (C₆-C₁₄aryl)alkyl-O—, optionally substituted with from 1 to 3 C₁-C₆alkoxy,
   (s) —C(O)NR₁₂R₁₂,
   (t) NHC(O)R₁₃,
   (u) —NHC(O)NR₁₂R₁₂,
   (v) —NHC(O)OR₁₃,
   (w) —NH(SO₂)—(C₁-C₆alkyl),
   (x) or —(SO₂)—(C₁-C₆alkyl);
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl.

In one embodiment, R₄ is hydrogen.

In some embodiments, R₉ is C₁-C₆alkyl.

In some embodiments, R₉ is (C₁-C₆alkoxy)carbonyl.

In some embodiments, R₉ is C₁-C₈acyl.

In some embodiments, R₉ is (C₆-C₁₄aryl)alkyl, wherein the ring portion of the (C₆-C₁₄aryl)alkyl group is optionally substituted by 1 to 3 substituents as specified in Formula IIb.

In some embodiments, R₉ is heteroaryl(C₁-C₆alkyl), wherein the ring portion of the heteroaryl(C₁-C₆alkyl) group is optionally substituted by 1 to 3 substituents as specified in Formula IIb.

In some embodiments, X₃ is —N—.

In other embodiments, X₃ is —C(H)—.

In some embodiments, R₁₁ is hydrogen.

In some embodiments, R₁₁ is hydroxyl.

In some embodiments, R₁₁ is —NR₁₂R₁₂.

In some embodiments, R₁₁ is —NHC(O)NR₁₂R₁₂.

In some embodiments, R₁₁ is —NHC(O)OR₁₃.

In some embodiments, one R₁₂ is hydrogen.

In some embodiments, one R₁₂ is C₁-C₆alkyl.

In some embodiments, one R₁₂ is C₆-C₁₄aryl.

In some embodiments, R₁₃ is one C₁-C₆alkyl.

Additional illustrative compounds of formula IIb are set forth below:

| Compound | |
|---|---|
| Number | Compound Name |
| 33 | {3-[9-(1-benzylpiperidin-4-yl)-6-morpholin-4-yl-9H-purin-2- |
| | yl]phenyl}methanol |
| 36 | 5-(6-morpholin-4-yl-9-piperidin-4-yl-9H-purin-2-yl)pyridin-3-ol |
| 37 | 5-(9-{1-[(5-methyl-2-thienyl)methyl]piperidin-4-yl}-6-morpholin-4-yl-9H- |
| | purin-2-yl)pyridin-3-ol |
| 38 | 5-{9-[1-(4-chlorobenzyl)piperidin-4-yl]-6-morpholin-4-yl-9H-purin-2- |
| | yl}pyridin-3-ol |
| 39 | 5-{6-morpholin-4-yl-9-[1-(1H-pyrrol-2-ylmethyl)piperidin-4-yl]-9H- |
| | purin-2-yl}pyridin-3-ol |
| 40 | 5-{9-[1-(4-methylbenzyl)piperidin-4-yl]-6-morpholin-4-yl-9H-purin-2- |
| | yl}pyridin-3-ol |
| 45 | (3-{9-[1-(2-fluorobenzyl)piperidin-4-yl]-6-morpholin-4-yl-9H-purin-2- |
| | yl}phenyl)methanol |
| 47 | (3-{6-morpholin-4-yl-9-[1-(4-pyridin-4-ylbenzyl)piperidin-4-yl]-9H- |
| | purin-2-yl}phenyl)methanol |
| 49 | (3-{9-[1-(2,4-difluorobenzyl)piperidin-4-yl]-6-morpholin-4-yl-9H- |
| | purin-2-yl}phenyl)methanol |
| 52 | (3-{9-[1-(2-furylmethyl)piperidin-4-yl]-6-morpholin-4-yl-9H-purin-2- |
| | yl}phenyl)methanol |
| 61 | [3-(9-{1-[(6-fluoropyridin-3-yl)methyl]piperidin-4-yl}-6-morpholin-4- |
| | yl-9H-purin-2-yl)phenyl]methanol |
| 62 | (3-{9-[1-(3,4-difluorobenzyl)piperidin-4-yl]-6-morpholin-4-yl-9H- |
| | purin-2-yl}phenyl)methanol |
| 63 | [3-(9-{1-[(6-chloropyridin-3-yl)methyl]piperidin-4-yl}-6-morpholin-4- |
| | yl-9H-purin-2-yl)phenyl]methanol |
| 68 | (3-{6-morpholin-4-yl-9-[1-(1H-pyrrol-2-ylmethyl)piperidin-4-yl]-9H- |
| | purin-2-yl}phenyl)methanol |
| 71 | [3-(9-{1-[(6-bromopyridin-2-yl)methyl]piperidin-4-yl}-6-morpholin-4- |
| | yl-9H-purin-2-yl)phenyl]methanol |
| 73 | [3-(9-{1-[(5-fluoro-1H-indol-3-yl)methyl]piperidin-4-yl}-6-morpholin- |
| | 4-yl-9H-purin-2-yl)phenyl]methanol |
| 75 | {3-[9-(1-{4-[3-dimethylamino)propoxy]benzyl}piperidin-4-yl)-6- |
| | morpholin-4-yl-9H-purin-2-yl]phenyl}methanol |
| 78 | 3-(9-{1-[(6-chloropyridin-3-yl)methyl]piperidin-4-yl}-6-morpholin-4- |
| | yl-9H-purin-2-yl)phenol |
| 81 | 3-(9-{1-[(6-bromopyridin-3-yl)methyl]piperidin-4-yl}-6-morpholin-4- |
| | yl-9H-purin-2-yl)phenol |

The following definitions are used in connection with the Imidazolopyrimidine Analogs unless the context indicates otherwise. In general, the number of carbon atoms present in a given group is designated “Cₓ-C_{y}”, where x and y are the lower and upper limits, respectively. For example, a group designated as “C₁-C₆” contains from 1 to 6 carbon atoms. The carbon number as used in the definitions herein refers to carbon backbone and carbon branching, but does not include carbon atoms of the substituents, such as alkoxy substitutions and the like.

“Acyl” refers to a carbonyl group bonded to a moiety comprising a hydrogen atom or from 1 to 8 carbon atoms in a straight, branched, or cyclic configuration or a combination thereof, attached to the parent structure through the carbonyl functionality. The moiety may be saturated or unsaturated, aliphatic or aromatic, and carbocyclic or heterocyclic. Examples of C₁-C₈acyl include acetyl-, acryl-, benzoyl-, nicotinoyl, isonicotinyl N-oxide, propionyl-, isobutyryl-, oxalyl-, and the like. An acyl group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, or C₃-C₈cycloalkyl.

“(Alkoxy)carbonyl” refers to the group alkyl-O—C(O)—. An (alkoxy)carbonyl group can be unsubstituted or substituted with one or more of the following groups: halogen, hydroxyl, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, C₁-C₆alkoxy, —C(O)OH, —C(O)O(C₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂. Exemplary (C₁-C₆alkoxy)carbonyl groups include but are not limited to CH₃—O—C(O)—, CH₃CH₂—O—C(O)—, CH₃CH₂CH₂—O—C(O)—, (CH₃)₂CH—O—C(O)—, and CH₃CH₂CH₂CH₂—O—C(O)—.

“Alkyl” refers to a hydrocarbon chain that may be a straight chain or branched chain, containing the indicated number of carbon atoms. For example, C₁-C₁₀ indicates that the group may have from 1 to 10 (inclusive) carbon atoms in it. In the absence of any numerical designation, “alkyl” is a chain (straight or branched) having 1 to 6 (inclusive) carbon atoms in it.

“C₁-C₃ alkyl” refers to a straight or branched chain saturated hydrocarbon containing 1-3 carbon atoms. Examples of a C₁-C₃ alkyl group include, but are not limited to, methyl, ethyl, propyl and isopropyl.

“C₁-C₄ alkyl” refers to a straight or branched chain saturated hydrocarbon containing 1-4 carbon atoms. Examples of a C₁-C₄ alkyl group include, but are not limited to, methyl, ethyl, propyl, butyl, isopropyl, isobutyl, sec-butyl and tert-butyl.

“C₁-C₅ alkyl” refers to a straight or branched chain saturated hydrocarbon containing 1-4 carbon atoms. Examples of a C₁-C₅ alkyl group include, but are not limited to, methyl, ethyl, propyl, butyl, pentyl, isopropyl, isobutyl, sec-butyl and tert-butyl, isopentyl and neopentyl.

“C₁-C₈ alkyl” refers to a straight or branched chain saturated hydrocarbon containing 1-8 carbon atoms. Examples of a C₁-C₈ alkyl group include, but are not limited to, methyl, ethyl, propyl, butyl, pentyl, hexyl, heptyl, octyl, isopropyl, isobutyl, sec-butyl and tert-butyl, isopentyl, neopentyl, isohexyl, isoheptyl and isooctyl.

“C₁-C₉ alkyl” refers to a straight or branched chain saturated hydrocarbon containing 1-9 carbon atoms. Examples of a C₁-C₉ alkyl group include, but are not limited to, methyl, ethyl, propyl, butyl, pentyl, hexyl, heptyl, octyl, nonyl, isopropyl, isobutyl, sec-butyl and tert-butyl, isopentyl, neopentyl, isohexyl, isoheptyl, isooctyl and isononyl.

“C₁-C₁₀ alkyl” refers to a straight or branched chain saturated hydrocarbon containing 1-10 carbon atoms. Examples of a C₁-C₁₀ alkyl group include, but are not limited to, methyl, ethyl, propyl, butyl, pentyl, hexyl, heptyl, octyl, nonyl, decyl, isopropyl, isobutyl, sec-butyl and tert-butyl, isopentyl, neopentyl, isohexyl, isoheptyl, isooctyl, isononyl and isodecyl.

“C₂-C₆alkenyl” refers to a straight or branched chain unsaturated hydrocarbon containing 2-6 carbon atoms and at least one double bond. Examples of a C₂-C₆alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, and isohexene.

“C₂-C₁₀alkenyl” refers to a straight or branched chain unsaturated hydrocarbon containing 2-10 carbon atoms and at least one double bond. Examples of a C₂-C₁₀alkenyl group include, but are not limited to, ethylene, propylene, 1-butylene, 2-butylene, isobutylene, sec-butylene, 1-pentene, 2-pentene, isopentene, 1-hexene, 2-hexene, 3-hexene, isohexene, 1-heptene, 2-heptene, 3-heptene, 1-octene, 2-octene, 3-octene, 4-octene, 1-nonene, 2-nonene, 3-nonene, 4-nonene, 1-decene, 2-decene, 3-decene, 4-decene and 5-decene.

“C₂-C₁₀alkynyl” refers to a straight or branched chain unsaturated hydrocarbon containing 2-10 carbon atoms and at least one triple bond. Examples of a C₂-C₁₀alkynyl group include, but are not limited to, acetylene, propyne, 1-butyne, 2-butyne, isobutyne, sec-butyne, 1-pentyne, 2-pentyne, isopentyne, 1-hexyne, 2-hexyne, 3-hexyne, isohexyne, 1-heptyne, 2-heptyne, 3-heptyne, 1-octyne, 2-octyne, 3-octyne, 4-octyne, 1-nonyne, 2-nonyne, 3-nonyne, 4-nonyne, 1-decyne, 2-decyne, 3-decyne, 4-decyne and 5-decyne.

“C₃-C₆alkynyl” refers to a straight or branched chain unsaturated hydrocarbon containing 3-6 carbon atoms and at least one triple bond. Examples of a C₃-C₆alkynyl group include, but are not limited to propyne, 1-butyne, 2-butyne, isobutyne, sec-butyne, 1-pentyne, 2-pentyne, isopentyne, 1-hexyne, 2-hexyne, 3-hexyne, and isohexyne.

“C₁-C₄alkylene” refers to a C₁-C₄alkyl group in which one of the C₁-C₄alkyl group's hydrogen atoms has been replaced with a bond. Examples of a C₁-C₄alkylene include —CH₂—, —CH₂CH₂—, —CH₂CH₂CH₂— and —CH₂CH₂CH₂CH₂—.

“C₁-C₅alkylene” refers to a C₁-C₅alkyl group in which one of the C₁-C₅ alkyl group's hydrogen atoms has been replaced with a bond. Examples of a C₁-C₄alkylene include —CH₂—, —CH₂CH₂—, —CH₂CH₂CH₂— and examples of a C₁-C₄ alkylene include —CH₂—, —CH₂CH₂—, —CH₂CH₂CH₂—, —CH₂CH₂CH₂CH₂— and ——CH₂CH₂CH₂CH₂CH₂—

“C₃-C₆alkylene” refers to a straight or branched chain unsaturated hydrocarbon containing 3-6 carbon atoms and at least one double bond. Examples of a C₃-C₆alkylene group include, but are not limited to propene, 1-butene, 2-butene, isobutene, sec-butene, 1-pentene, 2-pentene, isopentene, 1-hexene, 2-hexene, 3-hexene, and isohexene.

“Alkylcarboxy” refers to an alkyl group, defined above, attached to the parent structure through the oxygen atom of a carboxyl (C(O)—O—) functionality. Examples include acetoxy, ethylcarboxy, propylcarboxy, and isopentylcarboxy.

“Alkylhalo” refers to a C₁-C₅alkyl group, as defined above, wherein one or more of the C₁-C₅ alkyl group's hydrogen atoms has been replaced with —F, —Cl, —Br or —I. Representative examples of an alkylhalo group include, but are not limited to —CH₂F, —CCl₃, —CF₃, —CH₂Cl, —CH₂CH₂Br, —CH₂CH₂I, —CH₂CH₂CH₂F, —CH₂CH₂CH₂Cl, —CH₂CH₂CH₂CH₂Br, —CH₂CH₂CH₂CH₂I, —CH₂CH₂CH₂CH₂CH₂Br, —CH₂CH₂CH₂CH₂CH₂I, —CH₂CH(Br)CH₃, —CH₂CH(Cl)CH₂CH₃, —CH(F)CH₂CH₃ and —C(CH₃)₂(CH₂Cl).

“(Alkyl)amino-” refers to an —NH group, the nitrogen atom of said group being attached to a alkyl group, as defined above. Representative examples of an (C₁-C₆alkyl)amino group include, but are not limited to —NHCH₃, —NHCH₂CH₃, —NHCH₂CH₂CH₃, —NHCH₂CH₂CH₂CH₃, —NHCH(CH₃)₂, —NHCH₂CH(CH₃)₂, —NHCH(CH₃)CH₂CH₃ and —NH—C(CH₃)₃. An (alkyl)amino group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, C₁-C₆alkoxy, C₁-C₆alkyl, —C(O)OH, —C(O)O(C₁-C₆alkyl), —C(O)(C₁-C₆alkyl), C₆-C₁₄aryl, C₁-C₉heteroaryl, C₃-C₈cycloalkyl, C₁-C₆haloalkyl-, C₁-C₆aminoalkyl-, —OC(O)(C₁-C₆alkyl), C₁-C₆carboxyamidoalkyl-, or —NO₂.

“Aminoalkyl-” refers to an alkyl group, as defined above, wherein one or more of the alkyl group's hydrogen atoms has been replaced with —NH₂. Representative examples of an C₁-C₆aminoalkyl-group include, but are not limited to —CH₂NH₂, —CH₂CH₂NH₂, —CH₂CH₂CH₂NH₂, —CH₂CH₂CH₂CH₂NH₂, —CH₂CH(NH₂)CH₃, —CH₂CH(NH₂)CH₂CH₃, —CH(NH₂)CH₂CH₃ and —C(CH₃)₂(CH₂NH₂), —CH₂CH₂CH₂CH₂CH₂NH₂, and —CH₂CH₂CH(NH₂)CH₂CH₃. An aminoalkyl-group can be unsubstituted or substituted with one or two of the following groups C₁-C₆alkoxy, C₆-C₁₄aryl, C₁-C₉heteroaryl, C₃-C₈cycloalkyl, and C₁-C₆alkyl.

“Di(alkyl)amino-” refers to a nitrogen atom which has attached to it two alkyl groups, as defined above. Each alkyl group can be independently selected. Representative examples of an di(C₁-C₆alkyl)amino-group include, but are not limited to, —N(CH₃)₂, —N(CH₂CH₃)(CH₃), —N(CH₂CH₃)₂, —N(CH₂CH₂CH₃)₂, —N(CH₂CH₂CH₂CH₃)₂, —N(CH(CH₃)₂)₂, —N(CH(CH₃)₂)(CH₃), —N(CH₂CH(CH₃)₂)₂, —NH(CH(CH₃)CH₂CH₃)₂, —N(C(CH₃)₃)₂, —N(C(CH₃)₃)(CH₃), and —N(CH₃)(CH₂CH₃). The two alkyl groups on the nitrogen atom, when taken together with the nitrogen to which they are attached, can form a 3- to 7-membered nitrogen containing heterocycle wherein up to two of the carbon atoms of the heterocycle can be replaced with —N(R)—, —O—, or —S(O)ₚ—. R is hydrogen, C₁-C₆alkyl, C₃-C₈cycloalkyl, C₆-C₁₄aryl, C₁-C₉heteroaryl, C₁-C₆aminoalkyl-, or arylamino. Variable p is 0, 1, or 2.

“Aryl” refers to a phenyl or pyridyl group. Examples of an aryl group include, but are not limited to, phenyl, N-pyridyl, 2-pyridyl, 3-pyridyl and 4-pyridyl. An aryl group can be unsubstituted or substituted with one or more of the following groups: —C₁-C₅ alkyl, halo, -alkylhalo, hydroxyl, —C₁-C₅ alkylhydroxy, —NH₂, —aminoalkyl, -aminodialkyl, —COOH, —C(O)O—(C₁-C₅ alkyl), —OC(O)—(C₁-C₅ alkyl), —N-amidoalkyl, —C(O)NH₂, -carboxamidoalkyl, or —NO₂.

“Arylalkyl” refers to an aryl group, as defined above, wherein one of the aryl group's hydrogen atoms has been replaced with a C₁-C₅ alkyl group, as defined above. Representative examples of an arylalkyl group include, but are not limited to, 2-methylphenyl, 3-methylphenyl, 4-methylphenyl, 2-ethylphenyl, 3-ethylphenyl, 4-ethylphenyl, 2-propylphenyl, 3-propylphenyl, 4-propylphenyl, 2-butylphenyl, 3-butylphenyl, 4-butylphenyl, 2-pentylphenyl, 3-pentylphenyl, 4-pentylphenyl, 2-isopropylphenyl, 3-isopropylphenyl, 4-isopropylphenyl, 2-isobutylphenyl, 3-isobutylphenyl, 4-isobutylphenyl, 2-sec-butylphenyl, 3-sec-butylphenyl, 4-sec-butylphenyl, 2-t-butylphenyl, 3-t-butylphenyl and 4-t-butylphenyl.

“Arylamido” refers to an aryl group, as defined above, wherein one of the aryl group's hydrogen atoms has been replaced with one or more —C(O)NH₂ groups. Representative examples of an arylamido group include 2-C(O)NH₂-phenyl, 3-C(O)NH₂-phenyl, 4-C(O)NH₂-phenyl, 2-C(O)NH₂-pyridyl, 3-C(O)NH₂-pyridyl and 4-C(O)NH₂-pyridyl.

“(Aryl)alkyl” refers to an alkyl group, as defined above, wherein one or more of the alkyl group's hydrogen atoms has been replaced with a C₆-C₁₄aryl group as defined above. (C₆-C₁₄Aryl)alkyl moieties include benzyl, 1-phenylethyl, 2-phenylethyl, 3-phenylpropyl, 2-phenylpropyl, 1-naphthylmethyl, 2-naphthylmethyl and the like. An (aryl)alkyl group can be unsubstituted or substituted with one or more of the following groups: halogen, —NH₂, hydroxyl, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(Cl₁-C₆alkyl), —C(O)(C₁-C₆alkyl), C₆-C₁₄aryl, C₁-C₉heteroaryl, C₃-C₈cycloalkyl, haloalkyl-, aminoalkyl-, —OC(O)(C₁-C₆alkyl), C₁-C₆carboxyamidoalkyl-, or —NO₂.

“Alkylheterocycle” refers to a C₁-C₅ alkyl group, as defined above, wherein one of the C₁-C₅ alkyl group's hydrogen atoms has been replaced with a heterocycle. Representative examples of an alkylheterocycle group include, but are not limited to, —CH₂CH₂-morpholine, —CH₂CH₂-piperidine, —CH₂CH₂CH₂-morpholine and —CH₂CH₂CH₂-imidazole.

“Alkylamido” refers to a C₁-C₅ alkyl group, as defined above, wherein one of the C₁-C₅ alkyl group's hydrogen atoms has been replaced with a —C(O)NH₂ group. Representative examples of an alkylamido group include, but are not limited to, —CH₂C(O)NH₂, —CH₂CH₂C(O)NH₂, —CH₂CH₂CH₂C(O)NH₂, —CH₂CH₂CH₂CH₂C(O)NH₂, —CH₂CH₂CH₂CH₂CH₂C(O)NH₂, —CH₂CH(C(O)NH₂)CH₃, —CH₂CH(C(O)NH₂)CH₂CH₃, —CH(C(O)NH₂)CH₂CH₃ and —C(CH₃)2CH₂C(O)NH₂.

“Alkanol” refers to a C₁-C₅ alkyl group, as defined above, wherein one of the C₁-C₅ alkyl group's hydrogen atoms has been replaced with a hydroxyl group. Representative examples of an alkanol group include, but are not limited to, —CH₂OH, —CH₂CH₂OH, —CH₂CH₂CH₂OH, —CH₂CH₂CH₂CH₂OH, —CH₂CH₂CH₂CH₂CH₂OH, —CH₂CH(OH)CH₃, —CH₂CH(OH)CH₂CH₃, —CH(OH)CH₂CH₃ and —C(CH₃)2CH₂OH.

“Alkylcarboxy” refers to a C₁-C₅ alkyl group, as defined above, wherein one of the C₁-C₅ alkyl group's hydrogen atoms has been replaced with a —COOH group. Representative examples of an alkylcarboxy group include, but are not limited to, —CH₂COOH, —CH₂CH₂COOH, —CH₂CH₂CH₂COOH, —CH₂CH₂CH₂CH₂COOH, —CH₂CH(COOH)CH₃, —CH₂CH₂CH₂CH₂CH₂COOH, —CH₂CH(COOH)CH₂CH₃, —CH(COOH)CH₂CH₃ and —C(CH₃)2CH₂COOH.

“N-amidoalkyl” refers to a —NHC(O)— group in which the carbonyl carbon atom of said group is attached to a C₁-C₅ alkyl group, as defined above. Representative examples of a N-amidoalkyl group include, but are not limited to, —NHC(O)CH₃, —NHC(O)CH₂CH₃, —NHC(O)CH₂CH₂CH₃, —NHC(O)CH₂CH₂CH₂CH₃, —NHC(O)CH₂CH₂CH₂CH₂CH₃, —NHC(O)CH(CH₃)2, —NHC(O)CH₂CH(CH₃)2, —NHC(O)CH(CH₃)CH₂CH₃, —NHC(O)—C(CH₃)₃ and —NHC(O)CH₂C(CH₃)₃.

“Carboxamidoalkyl” refers to a —C(O)NH— group in which the nitrogen atom of said group is attached to a C₁-C₅ alkyl group, as defined above. Representative examples of a carboxamidoalkyl group include, but are not limited to, —C(O)NHCH₃, —C(O)NHCH₂CH₃, —C(O)NHCH₂CH₂CH₃, —C(O)NHCH₂CH₂CH₂CH₃, —C(O)NHCH₂CH₂CH₂CH₂CH₃, —C(O)NHCH(CH₃)₂, —C(O)NHCH₂CH(CH₃)₂, —C(O)NHCH(CH₃)CH₂CH₃, —C(O)NH—C(CH₃)₃ and —C(O)NHCH₂C(CH₃)₃.

A “C₃-C₈ Carbocycle” is a non-aromatic, saturated hydrocarbon ring containing 3-8 carbon atoms. Representative examples of a C₃-C₈ carbocycle include, but are not limited to, cyclopropyl, cyclobutyl, cyclopentyl, cyclohexyl, cycloheptyl and cyclooctyl. A C₃-C₈ carbocycle can be unsubstituted or independently substituted with one or more of the following groups: —C₁-C₅ alkyl, halo, -alkylhalo, hydroxyl, —O—C₁-C₅ alkyl, —NH₂, -aminoalkyl, -aminodialkyl, —COOH, —C(O)O—(C₁-C₅ alkyl), —OC(O)—(C₁-C₅ alkyl), —N-amidoalkyl, —C(O)NH₂, -carboxyamidoalkyl or —NO₂.

“Halo” or halogen is —F, —Cl, —Br or —I.

“Heteroaryl” refers to mono and bicyclic aromatic groups containing from 4 to 10 atoms and at least one heteroatom. Heteroatom as used in the term heteroaryl refers to oxygen, sulfur and nitrogen atoms. Examples of monocyclic heteroaryls include, but are not limited to, oxazinyl, thiazinyl, diazinyl, triazinyl, tetrazinyl, imidazolyl, tetrazolyl, isoxazolyl, furanyl, furazanyl, oxazolyl, thiazolyl, thiophenyl, pyrazolyl, triazolyl, and pyrimidinyl. Examples of bicyclic heteroaryls include but are not limited to, benzimidazolyl, indolyl, isoquinolinyl, indazolyl, quinolinyl, quinazolinyl, purinyl, benzisoxazolyl, 6,8-dihydro-5H-imidazo[2,1-c][1,4]oxazin-3-yl, benzoxazolyl, benzthiazolyl, benzodiazolyl, benzotriazolyl, isoindolyl and indazolyl.

“Heteroaryl(alkyl)” refers to an alkyl group, as defined above, wherein one or more of the alkyl group's hydrogen atoms has been replaced with a heteroaryl group as defined above. Heteroaryl(C₁-C₆alkyl) moieties include 2-pyridylmethyl, 2-thiophenylethyl, 3-pyridylpropyl, 2-quinolinylmethyl, 2-indolylmethyl, and the like. A heteroaryl(alkyl) group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), monocyclic C₁-C₆heterocycle, C₆-C₁₄aryl, C₁-C₉heteroaryl, or C₃-C₈cycloalkyl.

The term “N-morpholinyl” refers to the structure A:

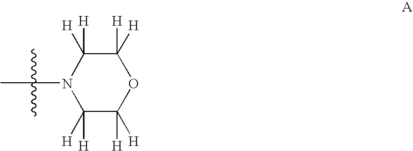

wherein any one or more of the eight morpholinyl hydrogen atoms can independently be substituted with C₁-C₆alkyl, C₂-C₆alkenyl, C₂-C₆alkynyl, C₁-C₃alkoxy, C₁-C₃acyl, C₁-C₃alkylcarboxy, —OH, (C₁-C₆alkyl)amino, halogen, ═O, or —CN.

The term “heteroatom” as used herein designates a sulfur, nitrogen, or oxygen atom.

“Heterocyclyl(alkyl)” refers to an alkyl group, as defined above, wherein one or more of the alkyl group's hydrogen atoms has been replaced with a heterocycle group. Heterocyclyl(C₁-C₆alkyl) moieties include 1-piperazinylethyl, 4-morpholinylpropyl, 6-piperazinylhexyl, and the like. A heterocyclyl(alkyl) group can be unsubstituted or substituted with one or more of the following groups: halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, hydroxyl, —O(C₁-C₆alkyl), C₁-C₆alkyl, —C(O)OH, —C(O)O(C₁-C₆alkyl), —C(O)(C₁-C₆alkyl), monocyclic C₁-C₆heterocycle, C₆-C₁₄aryl, C₁-C₉heteroaryl, or C₃-C₈cycloalkyl.

“Hydroxylalkyl-” refers to an alkyl group, as defined above, wherein one or more of the alkyl group's hydrogen atoms has been replaced with hydroxyl groups. Examples of C₁-C₆hydroxylalkyl-moieties include, for example, —CH₂OH, —CH₂CH₂OH, —CH₂CH₂CH₂OH, —CH₂CH(OH)CH₂OH, —CH₂CH(OH)CH₃, —CH(CH₃)CH₂OH and higher homologs.

“Perfluoroalkyl-” refers to a straight or branched chain hydrocarbon having two or more fluorine atoms. Examples of a C₁-C₆perfluoroalkyl-group include CF₃, CH₂CF₃, CF₂CF₃ and CH(CF₃)₂.

A “3- to 7-membered monocyclic heterocycle” refers to a monocyclic 3- to 7-membered non-aromatic monocyclic cycloalkyl in which 1-4 of the ring carbon atoms have been independently replaced with a N, O or S atom. Representative examples of a 3- to 7-membered monocyclic heterocycle group include, but are not limited to, morpholinyl, aziridine, oxirane, thiirane, pyrroline, pyrrolidine, dihydrofuran, tetrahydrofuran, dihydrothiophene, tetrahydrothiophene, dithiolane, piperidine, tetrahydropyran, pyran, thiane, thiine, piperazine, oxazine, thiazine, dithiane, dioxane, tetrahydroquinoline, and tetrahydroisoquinoline.

A “nitrogen containing 3- to 7-membered monocyclic heterocycle” refers to a monocyclic 3- to 7-membered non-aromatic monocyclic cycloalkyl group in which one of the cycloalkyl group's ring carbon atoms has been replaced with a nitrogen atom and 0-4 of the cycloalkyl group's remaining ring carbon atoms may be independently replaced with a N, O or S atom. Representative examples of nitrogen-containing-3- to 7-membered monocyclic heterocycles include, but are not limited to, piperidinyl, piperazinyl, aziridine, pyrroline, pyrrolidine, oxazine, thiazine, and morpholinyl. In one embodiment, a nitrogen containing 3- to 7-membered monocyclic heterocycle is substituted with up to three groups, independently chosen from: —C₁-C₅ alkyl, -halo, -halo-substituted C₁-C₅ alkyl, hydroxyl, —O—C₁-C₅ alkyl, —N(R^{a})₂, —COOH, —C(O)O—(C₁-C₅ alkyl), —OC(O)—(C₁-C₅ alkyl), —C(O)NH₂, or —NO₂, wherein each occurrence of R^{a} is independently —H, -benzyl, or —C₁-C₁₀ alkyl.

A “7- to 10-membered bicyclic heterocycle” refers to a bicyclic 7- to 10-membered non-aromatic bicyclic cycloalkyl in which 1-4 of the ring carbon atoms have been independently replaced with a N, O or S atom. Representative examples of a 7- to 10-membered bicyclic heterocycle group include, but are not limited to, azabicyclooctene, tetrahydroquinoline, tetrahydroisoquinoline, and indazolyl.

A “nitrogen-containing 7- to 10-membered bicyclic heterocycle” refers to a 7- to 10-membered bicyclic heterocycle, defined above, which contains at least one ring nitrogen atom. Representative nitrogen-containing 7- to 10-membered bicyclic heterocycles include azabicyclooctene, tetrahydroquinoline, tetrahydroisoquinoline, and indazolyl and the like.

The term “optionally substituted” as used herein means that at least one hydrogen atom of the optionally substituted group has been substituted with halogen, —NH₂—, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆ alkyl, —C(O)OH, —C(O)OC₁-C₆alkyl, —C(O)C₁-C₆, aryl, heteroaryl, or C₃-C₈ carbocycle.

A “subject” is a mammal, e.g., a human, mouse, rat, guinea pig, dog, cat, horse, cow, pig, or non-human primate, such as a monkey, chimpanzee, baboon or gorilla.

Representative “pharmaceutically acceptable salts” include, e.g., water-soluble and water-insoluble salts, such as the acetate, amsonate (4,4-diaminostilbene-2,2-disulfonate), benzenesulfonate, benzonate, bicarbonate, bisulfate, bitartrate, borate, bromide, butyrate, calcium edetate, camsylate, carbonate, chloride, citrate, clavulariate, dihydrochloride, edetate, edisylate, estolate, esylate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, and valerate salts.

The following abbreviations are used herein and have the indicated definitions: ACN is acetonitrile, AcOH is acetic acid, ATP is adenosine triphosphate, and BOC is t-butoxycarbonyl. Celite™ is flux-calcined diatomaceous earth. Celite™ is a registered trademark of World Minerals Inc. CHAPS is 3[(3-Cholamidopropyl)dimethylammonio]-propanesulfonic acid, DMF is N,N-dimethylformamide, DMSO is dimethylsulfoxide, DPBS is Dulbecco's Phosphate Buffered Saline Formulation, EDTA is ethylenediaminetetraacetic acid, ESI stands for Electrospray Ionization, EtOAc is ethyl acetate, EtOH is ethanol, HEPES is 4-(2-hydroxyethyl)-1-piperazineethanesulfonic acid, GMF is Glass microfiber, HPLC is high pressure liquid chromatography, LPS is lipopolysaccharide, MeCN is acetonitrile, MeOH is methanol, MS is mass spectrometry, NEt₃ is triethylamine, NMR is nuclear magnetic resonance, PBS is phosphate-buffered saline (pH 7.4), RPMI 1640 is a buffer (from Sigma-Aldrich Corp., St. Louis, Mo., USA), SDS is dodecyl sulfate (sodium salt), SRB is Sulforhodamine B, TCA is tricholoroacetic acid, TFA is trifluoroacetic acid, THF is tetrahydrofuran, TLC is thin-layer chromatography, and TRIS is Tris(hydroxymethyl)aminomethane.

The invention also includes pharmaceutical compositions comprising an effective amount of an Imidazolopyrimidine Analog and a pharmaceutically acceptable carrier. The invention includes an Imidazolopyrimidine Analog when provided as a pharmaceutically acceptable prodrug, hydrated salt, such as a pharmaceutically acceptable salt, or mixtures thereof

In other aspects, the compounds or pharmaceutically acceptable salts of the compounds of Formula (I), Formula (Ia), Formula (II), Formula (IIa) and Formula (IIb) are useful as pharmaceutical compositions comprising compounds or pharmaceutically acceptable salts of compounds of Formula (I), Formula (Ia), Formula (II), Formula (IIa) and Formula (IIb) and a pharmaceutically acceptable carrier.

In one aspect, the compounds or pharmaceutically acceptable salts of the compounds of Formula (I), Formula (Ia), Formula (II), Formula (IIa) and Formula (IIb) are useful as PI3K inhibitors.

In one aspect, the compounds or pharmaceutically acceptable salts of the compounds of Formula (I), Formula (Ia), Formula (II), Formula (IIa) and Formula (IIb) are useful as mTOR inhibitors.

In one embodiment, the invention provides methods for treating a PI3K-related disorder, comprising administering to a mammal in need thereof the compounds or pharmaceutically acceptable salts of compounds of Formula (I), Formula (Ia), Formula (II), Formula (IIa) and Formula (IIb) in an amount effective to treat a PI3K-related disorder.

In one embodiment, the invention provides methods for treating an mTOR-related disorder, comprising administering to a mammal in need thereof the compounds or pharmaceutically acceptable salts of compounds of Formula (I), Formula (Ia), Formula (II), Formula (IIa) and Formula (IIb) in an amount effective to treat an mTOR-related disorder.

An “effective amount” when used in connection an Imidazolopyrimidine Analog is an amount effective for treating or preventing a disease associated with PI3K or mTOR.

In other aspects, the invention provides methods of synthesizing the compounds or pharmaceutically acceptable salts of compounds of Formula (I), Formula (Ia), Formula (II), Formula (IIa) and Formula (IIb).

The Imidazolopyrimidine Analogs of the present invention exhibit a PI3K inhibitory activity and therefore, can be utilized in order to inhibit abnormal cell growth in which PI3K plays a role. Thus, the Imidazolopyrimidine Analogs are effective in the treatment of disorders with which abnormal cell growth actions of PI3K are associated, such as restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, cancer, etc. In particular, the Imidazolopyrimidine Analogs of the present invention possess excellent cancer cell growth inhibiting effects and are effective in treating cancers, preferably all types of solid cancers and malignant lymphomas, and especially, leukemia, skin cancer, bladder cancer, breast cancer, uterus cancer, ovary cancer, prostate cancer, lung cancer, colon cancer, pancreas cancer, renal cancer, gastric cancer, brain tumor, etc.

When administered to an animal, the Imidazolopyrimidine Analogs or pharmaceutically acceptable salts of the Imidazolopyrimidine Analogs can be administered neat or as a component of a composition that comprises a physiologically acceptable carrier or vehicle. A composition of the invention can be prepared using a method comprising admixing the Imidazolopyrimidine Analog or a pharmaceutically acceptable salt of the Imidazolopyrimidine Analog and a physiologically acceptable carrier, excipient, or diluent. Admixing can be accomplished using methods well known for admixing an Imidazolopyrimidine Analog or a pharmaceutically acceptable salt of the Imidazolopyrimidine Analog and a physiologically acceptable carrier, excipient, or diluent.

The present compositions, comprising Imidazolopyrimidine Analogs or pharmaceutically acceptable salts of the Imidazolopyrimidine Analogs of the invention can be administered orally. The Imidazolopyrimidine Analogs or pharmaceutically acceptable salts of Imidazolopyrimidine Analogs of the invention can also be administered by any other convenient route, for example, by infusion or bolus injection, by absorption through epithelial or mucocutaneous linings (e.g., oral, rectal, vaginal, and intestinal mucosa, etc.) and can be administered together with another therapeutic agent. Administration can be systemic or local. Various known delivery systems, including encapsulation in liposomes, microparticles, microcapsules, and capsules, can be used.

Methods of administration include, but are not limited to, intradermal, intramuscular, intraperitoneal, intravenous, subcutaneous, intranasal, epidural, oral, sublingual, intracerebral, intravaginal, transdermal, rectal, by inhalation, or topical, particularly to the ears, nose, eyes, or skin. In some instances, administration will result of release of the Imidazolopyrimidine Analog or a pharmaceutically acceptable salt of the Imidazolopyrimidine Analog into the bloodstream. The mode of administration is left to the discretion of the practitioner.

In one embodiment, the Imidazolopyrimidine Analog or a pharmaceutically acceptable salt of the Imidazolopyrimidine Analog is administered orally.

In another embodiment, the Imidazolopyrimidine Analog or a pharmaceutically acceptable salt of the Imidazolopyrimidine Analog is administered intravenously.

In another embodiment, it may be desirable to administer the Imidazolopyrimidine Analog or a pharmaceutically acceptable salt of the Imidazolopyrimidine Analog locally. This can be achieved, for example, by local infusion during surgery, topical application, e.g., in conjunction with a wound dressing after surgery, by injection, by means of a catheter, by means of a suppository or edema, or by means of an implant, said implant being of a porous, non-porous, or gelatinous material, including membranes, such as sialastic membranes, or fibers.

In certain embodiments, it can be desirable to introduce the Imidazolopyrimidine Analog or a pharmaceutically acceptable salt of the Imidazolopyrimidine Analog into the central nervous system, circulatory system or gastrointestinal tract by any suitable route, including intraventricular, intrathecal injection, paraspinal injection, epidural injection, enema, and by injection adjacent to the peripheral nerve. An intraventricular catheter, for example, can facilitate intraventricular injection attached to a reservoir, such as an Ommaya reservoir.

Pulmonary administration can also be employed, e.g., by use of an inhaler or nebulizer, and formulation with an aerosolizing agent, or via perfusion in a fluorocarbon or synthetic pulmonary surfactant. In certain embodiments, the Imidazolopyrimidine Analog or a pharmaceutically acceptable salt of the Imidazolopyrimidine Analog can be formulated as a suppository, with traditional binders and excipients such as triglycerides.

In another embodiment, the Imidazolopyrimidine Analog or a pharmaceutically acceptable salt of the Imidazolopyrimidine Analog can be delivered in a vesicle, in particular a liposome (see Langer, Science 249:1527-1533 (1990) and Treat et al., Liposomes in the Therapy of Infectious Disease and Cancer pp. 317-327 and pp. 353-365 (1989)).

In yet another embodiment, the Imidazolopyrimidine Analog or a pharmaceutically acceptable salt of the Imidazolopyrimidine Analog can be delivered in a controlled-release system or sustained-release system (see, e.g., Goodson, in Medical Applications of Controlled Release, vol. 2, pp. 115-138 (1984)). Other controlled or sustained-release systems discussed in the review by Langer, Science 249:1527-1533 (1990) can be used. In one embodiment, a pump can be used (Langer, Science 249:1527-1533 (1990); Sefton, CRC Crit. Ref. Biomed. Eng. 14:201 (1987); Buchwald et al., Surgery 88:507 (1980); and Saudek et al., N. Engl. J. Med. 321:574 (1989)). In another embodiment, polymeric materials can be used (see Medical Applications of Controlled Release (Langer and Wise eds., 1974); Controlled Drug Bioavailability, Drug Product Design and Performance (Smolen and Ball eds., 1984); Ranger and Peppas, J. Macromol. Sci. Rev. Macromol. Chem. 2:61 (1983); Levy et al., Science 228:190 (1935); During et al., Ann. Neural. 25:351 (1989); and Howard et al., J. Neurosurg. 71:105 (1989)).

In yet another embodiment, a controlled- or sustained-release system can be placed in proximity of a target of the Imidazolopyrimidine Analog or a pharmaceutically acceptable salt of the Imidazolopyrimidine Analog, e.g., the reproductive organs, thus requiring only a fraction of the systemic dose.

The present compositions can optionally comprise a suitable amount of a physiologically acceptable excipient.

Such physiologically acceptable excipients can be liquids, such as water and oils, including those of petroleum, animal, vegetable, or synthetic origin, such as peanut oil, soybean oil, mineral oil, sesame oil and the like. The physiologically acceptable excipients can be saline, gum acacia, gelatin, starch paste, talc, keratin, colloidal silica, urea and the like. In addition, auxiliary, stabilizing, thickening, lubricating, and coloring agents can be used. In one embodiment, the physiologically acceptable excipients are sterile when administered to an animal. The physiologically acceptable excipient should be stable under the conditions of manufacture and storage and should be preserved against the contaminating action of microorganisms. Water is a particularly useful excipient when the Imidazolopyrimidine Analog or a pharmaceutically acceptable salt of the Imidazolopyrimidine Analog is administered intravenously. Saline solutions and aqueous dextrose and glycerol solutions can also be employed as liquid excipients, particularly for injectable solutions. Suitable physiologically acceptable excipients also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The present compositions, if desired, can also contain minor amounts of wetting or emulsifying agents, or pH buffering agents.

Liquid carriers may be used in preparing solutions, suspensions, emulsions, syrups, and elixirs. The Imidazolopyrimidine Analog or pharmaceutically acceptable salt of the Imidazolopyrimidine Analog of this invention can be dissolved or suspended in a pharmaceutically acceptable liquid carrier such as water, an organic solvent, a mixture of both, or pharmaceutically acceptable oils or fat. The liquid carrier can contain other suitable pharmaceutical additives including solubilizers, emulsifiers, buffers, preservatives, sweeteners, flavoring agents, suspending agents, thickening agents, colors, viscosity regulators, stabilizers, or osmo-regulators. Suitable examples of liquid carriers for oral and parenteral administration include water (particular containing additives as above, e.g., cellulose Analogs, including sodium carboxymethyl cellulose solution), alcohols (including monohydric alcohols and polyhydric alcohols, e.g., glycols) and their Analogs, and oils (e.g., fractionated coconut oil and arachis oil). For parenteral administration the carrier can also be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellant.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the composition is in the form of a capsule. Other examples of suitable physiologically acceptable excipients are described in Remington's Pharmaceutical Sciences pp. 1447-1676 (Alfonso R. Gennaro, ed., 19th ed. 1995).

In one embodiment, the Imidazolopyrimidine Analog or a pharmaceutically acceptable salt of the Imidazolopyrimidine Analog is formulated in accordance with routine procedures as a composition adapted for oral administration to humans. Compositions for oral delivery can be in the form of tablets, lozenges, buccal forms, troches, aqueous or oily suspensions or solutions, granules, powders, emulsions, capsules, syrups, or elixirs for example. Orally administered compositions can contain one or more agents, for example, sweetening agents such as fructose, aspartame or saccharin; flavoring agents such as peppermint, oil of wintergreen, or cherry; coloring agents; and preserving agents, to provide a pharmaceutically palatable preparation. In powders, the carrier can be a finely divided solid, which is an admixture with the finely divided Imidazolopyrimidine Analog or pharmaceutically acceptable salt of the Imidazolopyrimidine Analog. In tablets, the Imidazolopyrimidine Analog or pharmaceutically acceptable salt of the Imidazolopyrimidine Analog is mixed with a carrier having the necessary compression properties in suitable proportions and compacted in the shape and size desired. The powders and tablets can contain up to about 99% of the Imidazolopyrimidine Analog or pharmaceutically acceptable salt of the Imidazolopyrimidine Analog.

Capsules may contain mixtures of the Imidazolopyrimidine Analogs or pharmaceutically acceptable salts of the Imidazolopyrimidine Analogs with inert fillers and/or diluents such as pharmaceutically acceptable starches (e.g., corn, potato, or tapioca starch), sugars, artificial sweetening agents, powdered celluloses (such as crystalline and microcrystalline celluloses), flours, gelatins, gums, etc.

Tablet formulations can be made by conventional compression, wet granulation, or dry granulation methods and utilize pharmaceutically acceptable diluents, binding agents, lubricants, disintegrants, surface modifying agents (including surfactants), suspending or stabilizing agents (including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrroldine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine.

Moreover, when in a tablet or pill form, the compositions can be coated to delay disintegration and absorption in the gastrointestinal tract, thereby providing a sustained action over an extended period of time. Selectively permeable membranes surrounding an osmotically active driving compound or a pharmaceutically acceptable salt of the compound are also suitable for orally administered compositions. In these latter platforms, fluid from the environment surrounding the capsule can be imbibed by the driving compound, which swells to displace the agent or agent composition through an aperture. These delivery platforms can provide an essentially zero order delivery profile as opposed to the spiked profiles of immediate release formulations. A time-delay material such as glycerol monostearate or glycerol stearate can also be used. Oral compositions can include standard excipients such as mannitol, lactose, starch, magnesium stearate, sodium saccharin, cellulose, and magnesium carbonate. In one embodiment, the excipients are of pharmaceutical grade.

In another embodiment, the Imidazolopyrimidine Analog or a pharmaceutically acceptable salt of the Imidazolopyrimidine Analog can be formulated for intravenous administration. Typically, compositions for intravenous administration comprise sterile isotonic aqueous buffer. Where necessary, the compositions can also include a solubilizing agent. Compositions for intravenous administration can optionally include a local anesthetic such as lignocaine to lessen pain at the site of the injection. Generally, the ingredients are supplied either separately or mixed together in unit dosage form, for example, as a dry lyophilized powder or water-free concentrate in a hermetically sealed container such as an ampoule or sachette indicating the quantity of active agent. Where the Imidazolopyrimidine Analog or a pharmaceutically acceptable salt of the Imidazolopyrimidine Analog is to be administered by infusion, it can be dispensed, for example, with an infusion bottle containing sterile pharmaceutical grade water or saline. Where the Imidazolopyrimidine Analog or a pharmaceutically acceptable salt of the Imidazolopyrimidine Analog is administered by injection, an ampoule of sterile water for injection or saline can be provided so that the ingredients can be mixed prior to administration.

In another embodiment, the Imidazolopyrimidine Analog or pharmaceutically acceptable salt of the Imidazolopyrimidine Analog can be administered transdermally through the use of a transdermal patch. Transdermal administrations include administrations across the surface of the body and the inner linings of the bodily passages including epithelial and mucosal tissues. Such administrations can be carried out using the present Imidazolopyrimidine Analogs or pharmaceutically acceptable salts of the Imidazolopyrimidine Analogs, in lotions, creams, foams, patches, suspensions, solutions, and suppositories (e.g., rectal or vaginal).

Transdermal administration can be accomplished through the use of a transdermal patch containing the Imidazolopyrimidine Analog or pharmaceutically acceptable salt of the Imidazolopyrimidine Analog and a carrier that is inert to the Imidazolopyrimidine Analog or pharmaceutically acceptable salt of the Imidazolopyrimidine Analog, is non-toxic to the skin, and allows delivery of the agent for systemic absorption into the blood stream via the skin. The carrier may take any number of forms such as creams or ointments, pastes, gels, or occlusive devices. The creams or ointments may be viscous liquid or semisolid emulsions of either the oil-in-water or water-in-oil type. Pastes comprised of absorptive powders dispersed in petroleum or hydrophilic petroleum containing the active ingredient may also be suitable. A variety of occlusive devices may be used to release the Imidazolopyrimidine Analog or pharmaceutically acceptable salt of the Imidazolopyrimidine Analog into the blood stream, such as a semi-permeable membrane covering a reservoir containing the Imidazolopyrimidine Analog or pharmaceutically acceptable salt of the Imidazolopyrimidine Analog with or without a carrier, or a matrix containing the active ingredient.

The Imidazolopyrimidine Analogs or pharmaceutically acceptable salts of the Imidazolopyrimidine Analogs of the invention may be administered rectally or vaginally in the form of a conventional suppository. Suppository formulations may be made from traditional materials, including cocoa butter, with or without the addition of waxes to alter the suppository's melting point, and glycerin. Water-soluble suppository bases, such as polyethylene glycols of various molecular weights, may also be used.

The Imidazolopyrimidine Analog or a pharmaceutically acceptable salt of the Imidazolopyrimidine Analog can be administered by controlled-release or sustained-release means or by delivery devices that are known to those of ordinary skill in the art. Such dosage forms can be used to provide controlled- or sustained-release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled- or sustained-release formulations known to those skilled in the art, including those described herein, can be readily selected for use with the active ingredients of the invention. The invention thus encompasses single unit dosage forms suitable for oral administration such as, but not limited to, tablets, capsules, gelcaps, and caplets that are adapted for controlled- or sustained-release. Advantages of controlled- or sustained-release compositions include extended activity of the drug, reduced dosage frequency, and increased compliance by the animal being treated. In addition, controlled- or sustained-release compositions can favorably affect the time of onset of action or other characteristics, such as blood levels of the Imidazolopyrimidine Analog or a pharmaceutically acceptable salt of the Imidazolopyrimidine Analog, and can thus reduce the occurrence of adverse side effects.

Controlled- or sustained-release compositions can initially release an amount of the Imidazolopyrimidine Analog or a pharmaceutically acceptable salt of the Imidazolopyrimidine Analog that promptly produces the desired therapeutic or prophylactic effect, and gradually and continually release other amounts of the Imidazolopyrimidine Analog or a pharmaceutically acceptable salt of the Imidazolopyrimidine Analog to maintain this level of therapeutic or prophylactic effect over an extended period of time. To maintain a constant level of the Imidazolopyrimidine Analog or a pharmaceutically acceptable salt of the Imidazolopyrimidine Analog in the body, the Imidazolopyrimidine Analog or a pharmaceutically acceptable salt of the Imidazolopyrimidine Analog can be released from the dosage form at a rate that will replace the amount of the Imidazolopyrimidine Analog or a pharmaceutically acceptable salt of the Imidazolopyrimidine Analog being metabolized and excreted from the body. Various conditions, including but not limited to, changes in pH, changes in temperature, concentration or availability of enzymes, concentration or availability of water, or other physiological conditions or Imidazolopyrimidine Analogs can stimulate controlled- or sustained-release of an active ingredient.

In certain embodiments, the present invention is directed to prodrugs of the Imidazolopyrimidine Analogs or pharmaceutically acceptable salts of Imidazolopyrimidine Analogs of the present invention. Various forms of prodrugs are known in the art, for example as discussed in Bundgaard (ed.), Design of Prodrugs, Elsevier (1985); Widder et al. (ed.), Methods in Enzymology, vol. 4, Academic Press (1985); Kgrogsgaard-Larsen et al. (ed.); “Design and Application of Prodrugs”, Textbook of Drug Design and Development, Chapter 5, 113-191 (1991); Bundgaard et al., Journal of Drug Delivery Reviews, 8:1-38 (1992); Bundgaard et al., J. Pharmaceutical Sciences, 77:285 et seq. (1988); and Higuchi and Stella (eds.), Prodrugs as Novel Drug Delivery Systems, American Chemical Society (1975).

The amount of the Imidazolopyrimidine Analog or a pharmaceutically acceptable salt of the Imidazolopyrimidine Analog that is effective for treating or preventing a PI3K-related disorder. In addition, in vitro or in vivo assays can optionally be employed to help identify optimal dosage ranges. The precise dose to be employed can also depend on the route of administration, the condition, the seriousness of the condition being treated, as well as various physical factors related to the individual being treated, and can be decided according to the judgment of a health-care practitioner. Equivalent dosages may be administered over various time periods including, but not limited to, about every 2 hours, about every 6 hours, about every 8 hours, about every 12 hours, about every 24 hours, about every 36 hours, about every 48 hours, about every 72 hours, about every week, about every two weeks, about every three weeks, about every month, and about every two months. The number and frequency of dosages corresponding to a completed course of therapy will be determined according to the judgment of a health-care practitioner. The effective dosage amounts described herein refer to total amounts administered; that is, if more than one Imidazolopyrimidine Analog or a pharmaceutically acceptable salt of the Imidazolopyrimidine Analog is administered, the effective dosage amounts correspond to the total amount administered.

The amount of the Imidazolopyrimidine Analog or a pharmaceutically acceptable salt of the Imidazolopyrimidine Analog that is effective for treating or preventing a PI3K-related disorder will typically range from about 0.001 mg/kg to about 250 mg/kg of body weight per day, in one embodiment, from about 1 mg/kg to about 250 mg/kg body weight per day, in another embodiment, from about 1 mg/kg to about 50 mg/kg body weight per day, and in another embodiment, from about 1 mg/kg to about 20 mg/kg of body weight per day.

In one embodiment, the pharmaceutical composition is in unit dosage form, e.g., as a tablet, capsule, powder, solution, suspension, emulsion, granule, or suppository. In such form, the composition is sub-divided in unit dose containing appropriate quantities of the active ingredient; the unit dosage form can be packaged compositions, for example, packeted powders, vials, ampoules, prefilled syringes or sachets containing liquids. The unit dosage form can be, for example, a capsule or tablet itself, or it can be the appropriate number of any such compositions in package form. Such unit dosage form may contain from about 1 mg/kg to about 250 mg/kg, and may be given in a single dose or in two or more divided doses.

The Imidazolopyrimidine Analog or a pharmaceutically acceptable salt of the Imidazolopyrimidine Analog can be assayed in vitro or in vivo for the desired therapeutic or prophylactic activity prior to use in humans. Animal model systems can be used to demonstrate safety and efficacy.

The present methods for treating or preventing a PI3K-related disorder, can further comprise administering another therapeutic agent to the animal being administered the Imidazolopyrimidine Analog or a pharmaceutically acceptable salt of the Imidazolopyrimidine Analog. In one embodiment, the other therapeutic agent is administered in an effective amount.

Effective amounts of the other therapeutic agents are well known to those skilled in the art. However, it is well within the skilled artisan's purview to determine the other therapeutic agent's optimal effective amount range. The Imidazolopyrimidine Analog or a pharmaceutically acceptable salt of the Imidazolopyrimidine Analog and the other therapeutic agent can act additively or, in one embodiment, synergistically. In one embodiment, of the invention, where another therapeutic agent is administered to an animal, the effective amount of the Imidazolopyrimidine Analog or a pharmaceutically acceptable salt of the Imidazolopyrimidine Analog is less than its effective amount would be where the other therapeutic agent is not administered. In this case, without being bound by theory, it is believed that the Imidazolopyrimidine Analog or a pharmaceutically acceptable salt of the Imidazolopyrimidine Analog and the other therapeutic agent act synergistically.

In one embodiment, the Imidazolopyrimidine Analog or a pharmaceutically acceptable salt of the Imidazolopyrimidine Analog is administered concurrently with another therapeutic agent.

In one embodiment, a composition comprising an effective amount of the Imidazolopyrimidine Analog or a pharmaceutically acceptable salt of the Imidazolopyrimidine Analog and an effective amount of another therapeutic agent within the same composition can be administered.

In another embodiment, a composition comprising an effective amount of the Imidazolopyrimidine Analog or a pharmaceutically acceptable salt of the Imidazolopyrimidine Analog and a separate composition comprising an effective amount of another therapeutic agent can be concurrently administered. In another embodiment, an effective amount of the Imidazolopyrimidine Analog or a pharmaceutically acceptable salt of the Imidazolopyrimidine Analog is administered prior to or subsequent to administration of an effective amount of another therapeutic agent. In this embodiment, the Imidazolopyrimidine Analog or a pharmaceutically acceptable salt of the Imidazolopyrimidine Analog is administered while the other therapeutic agent exerts its therapeutic effect, or the other therapeutic agent is administered while the Imidazolopyrimidine Analog or a pharmaceutically acceptable salt of the Imidazolopyrimidine Analog exerts its preventative or therapeutic effect for treating or preventing a PI3K-related disorder.

Suitable other therapeutic agents useful in the methods and compositions of the present invention include, but are not limited to temozolomide, a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, taxanes such as docetaxel and paclitaxel, leucovorin, levamisole, irinotecan, estramustine, etoposide, nitrogen mustards, BCNU, nitrosoureas such as carmustine and lomustine, vinca alkaloids such as vinblastine, vincristine and vinorelbine, platinum complexes such as cisplatin, carboplatin and oxaliplatin, imatinib mesylate, Avastin (Bevacizumab), hexamethylmelamine, topotecan, tyrosine kinase inhibitors, tyrphostins, herbimycin A, genistein, erbstatin, and lavendustin A.

Other therapeutic agents useful in the methods and compositions of the present invention include, but are not limited to hydroxyzine, glatiramer acetate, interferon beta-1a, interferon beta-1b, mitoxantrone, and natalizumab.

In another embodiment, the pharmaceutically acceptable carrier is suitable for oral administration and the composition comprises an oral dosage form.

The Imidazolopyrimidine Analogs and pharmaceutically acceptable salts of Imidazolopyrimidine Analogs can be prepared using a variety of methods starting from commercially available compounds, known compounds, or compounds prepared by known methods. General synthetic routes to many of the compounds of the invention are included in the following schemes. It is understood by those skilled in the art that protection and deprotection steps not shown in the Schemes may be required for these syntheses, and that the order of steps may be changed to accommodate functionality in the target molecule.

Schemes 1-8 demonstrate the synthesis of compounds and pharmaceutically acceptable salts of the compounds of Formulas (I), (Ia), (II), (IIa), and (IIb).

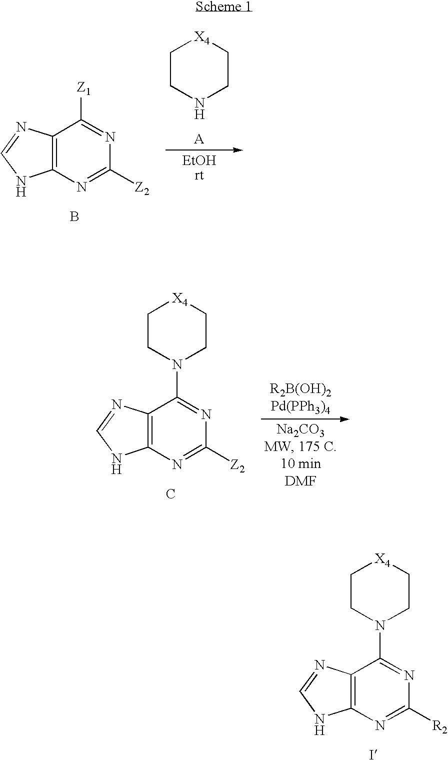

wherein X₄ is —O—, —CH₂—, —N(H)—, S(O)ₙ wherein n is 0, 1, or 2; Z₁, and Z₂ are each independently halogen and R₂ is as defined above.

The synthesis of the desired Imidazolopyrimidine Analogs may be prepared according to Scheme 1 by first reacting morpholine A with commercially available dichloropurine B in EtOH then subjecting the resulting pyrimidylchloride C to Suzuki reaction with boronic acids D under either microwave or thermal conditions to give product E. The boronic acids are commercially available or can be prepared synthetically via standard organic chemistry protocols.

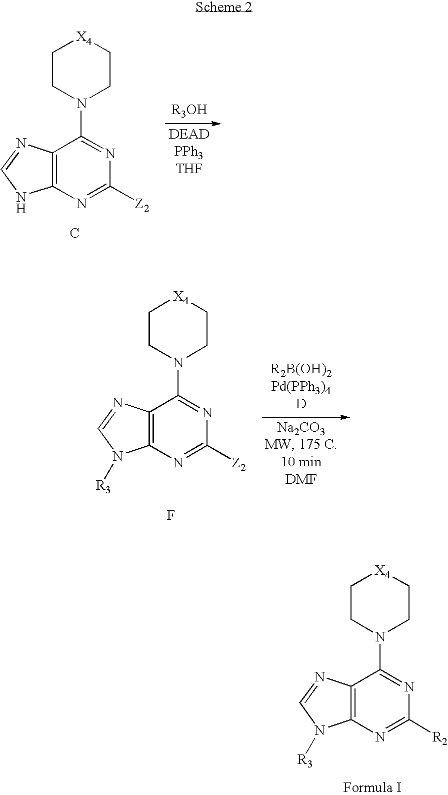

wherein X₄ and Z₂ are as defined in Scheme 1, and R₂ and R₃ are as defined above.

The substituted Imidazolopyrimidine Analogs may be prepared according to Scheme 2 by first reacting the synthesized morpholine intermediate C with alcohols under standard Mitsunobu reaction conditions. The resulting halogenated pyrimidine F is then subjected to Suzuki reaction with boronic acids D under microwave conditions to give an R₂ substituted compound of Formula I. The alcohols, boronic acids, and electrophiles are commercially available or can be prepared synthetically via standard organic chemistry protocols. In a more specific example, the monomorpholinyl intermediate C can be reacted with N-t-BOC protected piperidine alcohol under standard Mitsunobu reaction. The t-BOC can be removed after the Suzuki coupling and the liberated basic nitrogen can be alkylated using an alkyl halide. The piperidinyl nitrogen can also be alkylated using a reductive amination procedure using various aldehydes or ketones in the presence of NaCNBH₃ and ZnBr₂ as depicted in Scheme 3.

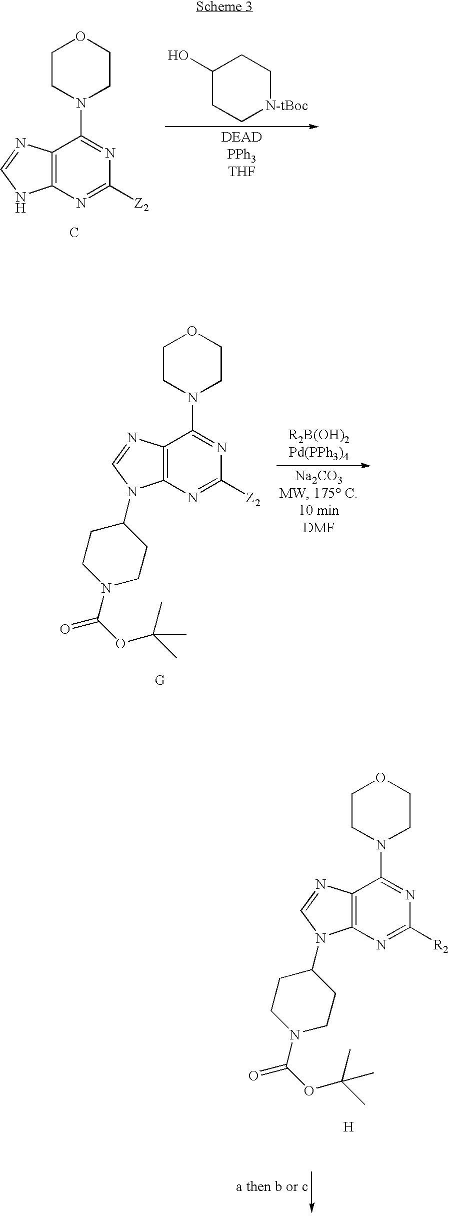

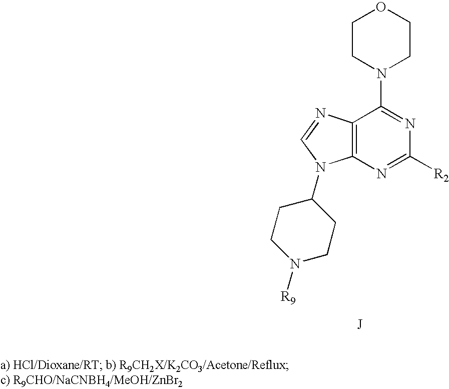

wherein Z₂ is as defined in Scheme 1, and R₂ and R₉ are as defined above.

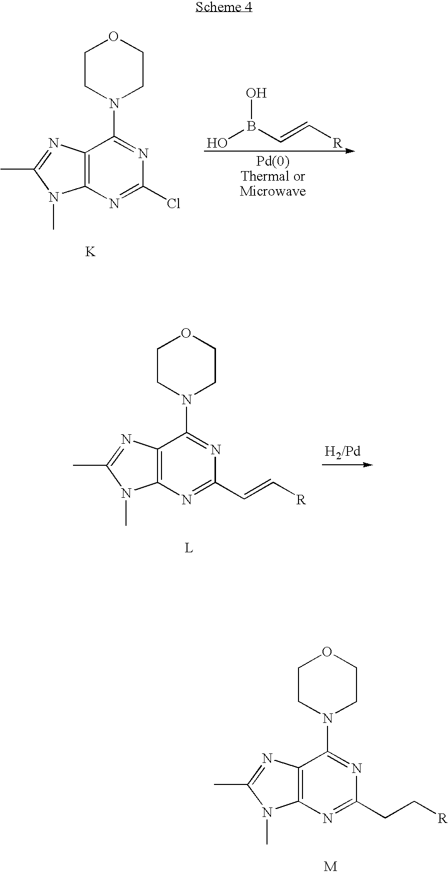

As set forth in Scheme 4, a compound of formula L can be formed by reaction of a compound of formula K with an vinyl boronic acid under Suzuki coupling reaction conditions such as Pd(0) catalyst in an organic solvent such as dimethoxy ethane or ethanol/toluene mixture at 80° C.-180° C. If desired the alkene compound of formula L can be further reduced to the alkyl substituent by treatment with Pd catalyst under a hydrogen atmosphere.

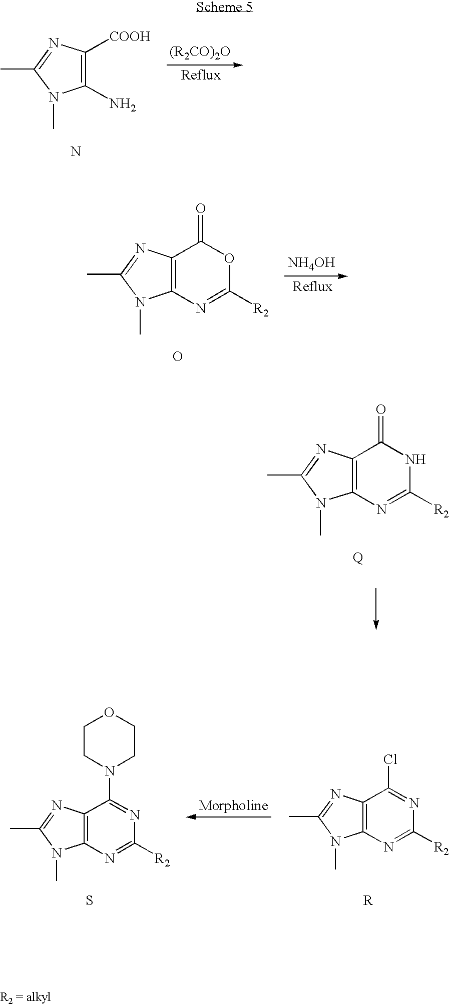

As set forth in Scheme 5, a compound of formula S can be obtained wherein R₂ is an alkyl substituent by first reacting a compound of formula N under reflux with a alkyl anhydride to give an isolatable intermediate compound of formula O. Further reflux in ammonium hydroxide gives a compound of formula Q that can be converted to the chloride using POCl₃. The chloride of a compound of formula R can be substituted with a morpholine type compound such as, for example, N morpholine to give a compound of formula S.

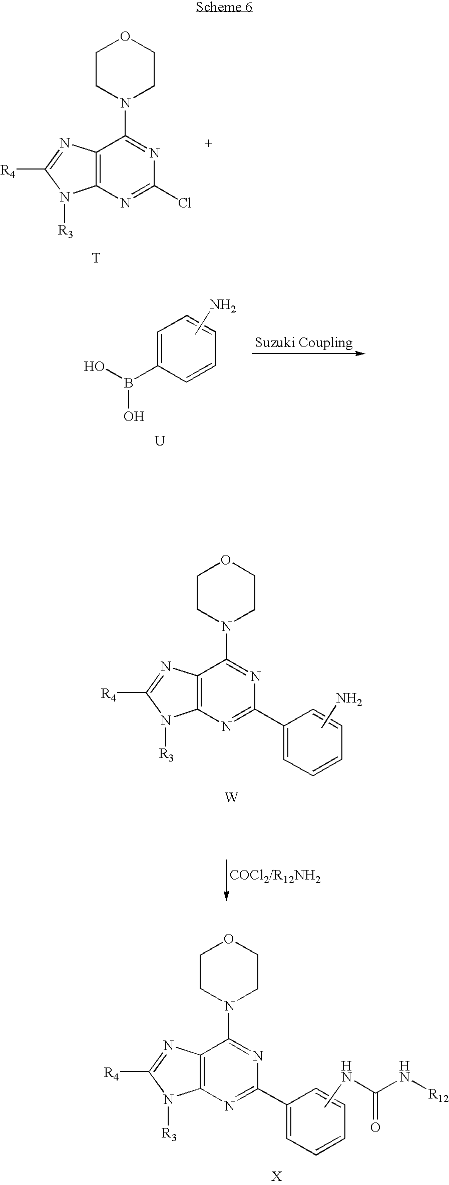

wherein R₃, R₄ and R₁₂ are as described above.

As set forth in scheme 6, an aryl urea compound of formula U can be synthesized by first reacting a compound of formula T with an aminoaryl boronic acid of the formula U under Suzuki coupling reaction conditions to give the isolatable synthetic intermediate compound of formula W. Reaction of a compound of formula W with an amine in the presence of phosgene will give a compound of the formula X.

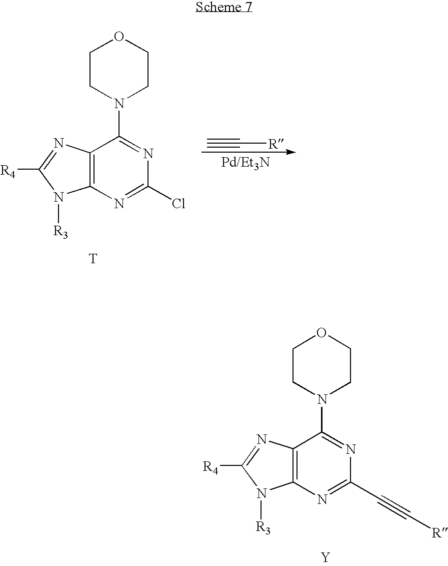

wherein R₃ and R₄ are as defined above and R″ is any group compatible with the conditions under which aryl chlorides are coupled to alkynes under palladium catalysis.

As set forth in Scheme 7, an alkyne compound of the formula Y can be obtained by reacting a compound of formula T with an alkyne in the presence of a Pd catalyst and triethylamine.

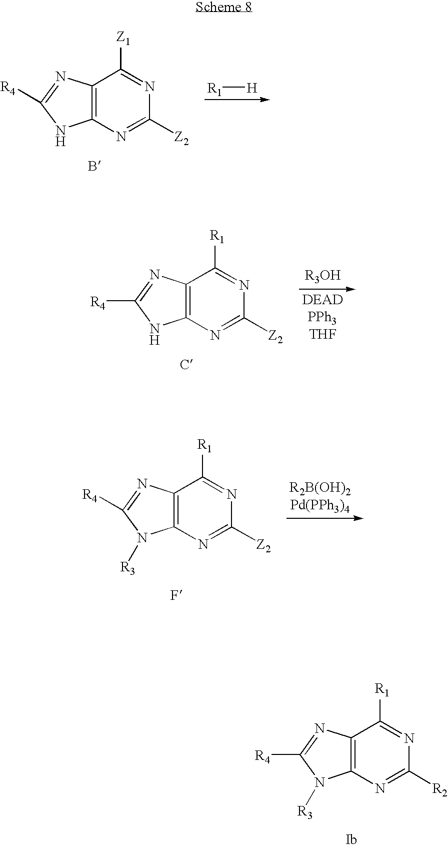

wherein Z₁, and Z₂ are each independently halogen and R₁—R₄ are as defined above in Formula Ib.

The synthesis of the desired Imidazolopyrimidine Analogs (Ib) may be prepared according to Scheme 8 by first reacting amine R₁—H with available dichloropurine B′ then subjecting the resulting purinyl chloride C′ with alcohols R₃OH under standard Mitsunobu reaction conditions. Suzuki reaction with boronic acids R₂B(OH)₂ under either microwave or thermal conditions gives product Ib. The boronic acids are commercially available or can be prepared synthetically via standard organic chemistry protocols. The starting purine compounds of Formula B′, used in Reaction Scheme 8, were obtained from either commercial sources or prepared by well-known literature procedures.

The general procedures used to synthesize the compounds of Formula I are described in Reaction Schemes 1-8 and are illustrated in the examples. Reasonable variations of the described procedures, which would be evident to one skilled in the art, are intended to be within the scope of the present invention.

The compounds herein described may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials.

### EXAMPLES

### General Methods

The following general methods outline the synthesis the Imidazolopyrimidine Analogs of the Examples.

### Synthesis of 6-Morpholin-4-yl-2-Aryl-9H-purine (Scheme 1)

Step 1: To a solution of the 2,6-dichloropurine (0.8 g, 4.23 mmol) dissolved in EtOH (40 mL) in is added morpholine (1.5 eq). The reaction is stirred for 12 hr at room temperature and the crude solid product filtered off. The crude product is washed with Et₂O and dried in vacuo affording 0.75 g of a beige solid.

To the morpholine product of Step 1 (50 mg, 0.21 mmol) dissolved in DMF (0.5 mL) is added the desired aryl boronic acid (1.5 eq), Na₂CO₃ solution (2 eq), and Pd(PPh₃)₄ (catalytic amount) to a microwave conical vial. The reaction is heated under MW irradiation at 175° C. for 10 minutes. The crude reaction is then concentrated and purified via preparative HPLC using a Gilson instrument (see below).

### Synthesis of 6-Morpholin-4-yl-2-Aryl-9-piperidin-4-yl-9H-purine (Scheme 2)

Step 2: To the desired 1-Benzyl-4-hydroxypiperidine (1.14 g, 5.97 mmol) and PPh₃ (1.6 g, 5.96 mmol) dissolved in THF (20 mL) is added DEAD (0.94 mL, 5.97 mmol). The mixture is stirred for 30 min. and the 2-chloro-6-morpholino purine (obtained from the step 1), (0.95 g, 3.98 mmol) in THF (10 mL) is added. The reaction is stirred for 72 hr, concentrated, and purified via silica gel chromatography (10% MeOH/EtOAc) affording a yellow solid.

To the yellow solid obtained above (100 mg, 0.24 mmol dissolved in DMF (1 mL)) is added the desired aryl boronic acid (1.5 eq), Na₂CO₃ solution (2 eq), and Pd(PPh₃)₄ (catalytic amount) in a microwave conical vial. The reaction is heated under MW irradiation at 175° C. for 10 minutes. The crude reaction then concentrated, utilized in the next step, or the desired product is purified via preparative HPLC using a Gilson instrument (see below).

To a solution of the benzyl piperidinyl substrate (˜100 mgs) dissolved in a MeOH/4% formic acid solution (5 mL) is added Pd/C (100 mgs). The mixture is stirred for 24 hrs, filtered, and the crude reaction concentrated to be utilized directly in the next step or is purified via preparative HPLC using a Gilson instrument (see below).

To a solution of the free NH piperidinyl substrate (0.103 mmol) dissolved in THF (3 mL) is added TEA (22 μL, 0.15 mmol) and acetyl chloride (8 μL, 0.103 mmol). The mixture is heated to 50° C. and stirred for 24 hrs. The crude reaction mixture is concentrated and purified via preparative HPLC using a Gilson instrument (see below).

The following HPLC and LC/MS methods were used for the analysis of the products of the syntheses outlined in the Examples.

### Method A: Gilson Instrument

The Gilson crude material is dissolved in 1.5 ml DMSO 0.5 ml MeCN, filtered through a 0.45 μm GMF, and purified on a Gilson HPLC using a Phenomenex LUNA C₁₈ column: 60 mm×21.20 mm I.D., 5 μm particle size: with ACN/water (containing 0.2% TFA or Et₃N) gradient elution. The appropriate fractions are analyzed by LC/MS as described below. Combining pure fractions and evaporating the solvent in a Speed-Vac isolates the title compound.

### Method B

Instrument: HP Agilent 1100 LC/MS; UV Detector: Agilent 1100 Diode Array Detector; Mass Spectrometer Detector: Agilent MSD; Column: Waters Xterra MS C18 30 mm×2.1 mm i.d., 3.5 um; Flow Rate: 1.00 ml/min; Run Time: 5.00 min; Gradient Elution: 0 min 90% water, 10% acetonitrile; 3 min 10% water, 90% acetonitrile; Column Temperature: 50° C.; UV Signals: 215 nm, 254 nm; MS Parameters: Mass Range 100-1000, Fragmentor 140, Gain EMV 1.0.

The following Imidazolopyrimidine Analogs were prepared according to the above procedures.

TABLE 1

| Compound | | LC/MS MW | | Ret. | HPLC^{a,b} |
|---|---|---|---|---|---|
| No. | Name | Obs. | Mol Ion | Time | Conditions |
| 1 | 6-morpholin-4-yl-2-(2-thienyl)-9H-purine | 288.1 | M + H | 2.16 | std method |
| | | | | | w/formic |
| 2 | 6-morpholin-4-yl-2-(3-thienyl)-9H-purine | 288.1 | M + H | 2.1 | std method |
| | | | | | w/formic |
| 3 | 2-(6-morpholin-4-yl-9H-purin-2-yl)phenol | 298.1 | M + H | 2.28 | std method |
| | | | | | w/formic |
| 4 | 4-(6-morpholin-4-yl-9H-purin-2-yl)phenol | 298.1 | M + H | 1.79 | std method |
| | | | | | w/formic |
| 5 | [4-(6-morpholin-4-yl-9H-purin-2-yl)phenyl]methanol | 312.1 | M + H | 1.83 | std method |
| | | | | | w/formic |
| 6 | 2-(1H-indol-5-yl)-6-morpholin-4-yl-9H-purine | 321.1 | M + H | 1.92 | std method |
| | | | | | w/formic |
| 7 | 2-(1,3-benzodioxol-5-yl)-6-morpholin-4-yl-9H-purine | 326.1 | M + H | 2.17 | std method |
| | | | | | w/formic |
| 8 | 6-morpholin-4-yl-2-(3-nitrophenyl)-9H-purine | 325.1 | M − H | 2.31 | std method |
| | | | | | w/formic |
| 9 | 2-(1-benzothien-3-yl)-6-morpholin-4-yl-9H-purine | 338.1 | M + H | 2.43 | std method |
| | | | | | w/formic |
| 10 | 6-morpholin-4-yl-2-[3-(trifluoromethyl)phenyl]-9H- | 350.1 | M + H | 2.47 | std method |
| | purine | | | | w/formic |
| 11 | 2-(3-methylphenyl)-6-morpholin-4-yl-9H-purine | 296.1 | M + H | 2.27 | std method |
| | | | | | w/formic |
| 12 | 2-(3-isopropylphenyl)-6-morpholin-4-yl-9H-purine | 324.2 | M + H | 2.48 | std method |
| | | | | | w/formic |
| 13 | 3-(6-morpholin-4-yl-9H-purin-2-yl)benzonitrile | 307.1 | M + H | 2.2 | std method |
| | | | | | w/formic |
| 14 | 2-biphenyl-3-yl-6-morpholin-4-yl-9H-purine | 358.2 | M + H | 2.58 | std method |
| | | | | | w/formic |
| 15 | N-[3-(6-morpholin-4-yl-9H-purin-2- | 375.1 | M + H | 1.92 | std method |
| | yl)phenyl]methanesulfonamide | | | | w/formic |
| 16 | 6-morpholin-4-yl-2-(2-phenoxyphenyl)-9H-purine | 374.2 | M + H | 2.28 | std method |
| | | | | | w/formic |
| 17 | N,N-dimethyl-4-(6-morpholin-4-yl-9H-purin-2- | 353.2 | M + H | 1.94 | std method |
| | yl)benzamide | | | | w/formic |
| 18 | 2-(2,3-dihydro-1,4-benzodioxin-6-yl)-6-morpholin-4- | 340.1 | M + H | 2.09 | std method |
| | yl-9H-purine | | | | w/formic |
| 19 | 2-(3-furyl)-6-morpholin-4-yl-9H-purine | 272.1 | M + H | 1.93 | std method |
| | | | | | w/formic |
| 20 | 2-[3-(methylsulfonyl)phenyl]-6-morpholin-4-yl-9H- | 360.1 | M + H | 1.96 | std method |
| | purine | | | | w/formic |
| 21 | 3-(6-Piperidin-1-yl-9H-purin-2-yl)-phenol | 296.1 | M + H | 2.03 | std method |
| | | | | | w/formic |
| 22 | 2-(4-Methanesulfonyl-phenyl)-6-morpholin-4-yl-9H- | 360.1 | M + H | 1.94 | std method |
| | purine | | | | w/formic |
| 23 | 2-[3-(3,5-Dimethoxy-benzyloxy)-phenyl]-6- | 448.2 | M + H | 2.44 | std method |
| | morpholin-4-yl-9H-purine | | | | w/formic |
| 24 | 2-(4-Benzyloxy-3-chloro-phenyl)-6-morpholin-4-yl- | 422.1 | M + H | 2.62 | std method |
| | 9H-purine | | | | w/formic |
| 25 | [3-(6-Morpholin-4-yl-9-piperidin-4-yl-9H-purin-2- | 395.2 | M + H | 1.69 | std method |
| | yl)-phenyl]-methanol | | | | w/formic |
| 26 | 1-(4-(2-(3-(hydroxymethyl)phenyl)-6-morpholino- | 437.2 | M + H | 2.03 | std method |
| | 9H-purin-9-yl)piperidin-1-yl)ethanone | | | | w/formic |
| 27 | 3-(9-((1-benzylpiperidin-4-yl)methyl)-6-morpholino- | 485.3 | M + H | 1.93 | std method |
| | 9H-purin-2-yl)phenol | | | | w/formic |
| 28 | 3-(9-(1-benzylpiperidin-4-yl)-6-morpholino-9H- | 471.2 | M + H | 1.89 | std method |
| | purin-2-yl)phenol | | | | w/formic |
| 29 | (3-(9-((1-benzylpiperidin-4-yl)methyl)-6-morpholino- | 499.3 | M + H | 1.91 | std method |
| | 9H-purin-2-yl)phenyl)methanol | | | | w/formic |
| 30 | (3-(9-(1-benzylpiperidin-4-yl)-6-morpholino-9H- | 485.3 | M + H | 1.9 | std method |
| | purin-2-yl)phenyl)methanol | | | | w/formic |
| 53 | 4-(2-(3-methoxyphenyl)-9H-purin-6-yl)morpholine | 312 | M + H | 2.24 | Std method |
| | | | | | W/formic |
| 54 | 4-(2-phenyl-9H-purin-6-yl)morpholine | 282 | M + H | 2.03 | Std method |
| | | | | | W/formic |
| 55 | 3-(6-morpholino-9H-purin-2-yl)phenol | 298 | M + H | 1.77 | Std method |
| | | | | | W/formic |

^{a}HPLC Conditions: Instrument - Agilent 1100
Column: Thermo Aquasil C18, 50 × 2.1 mm, and 5 μm
Mobile Phase A: 0.1% Formic Acid in water; B: 0.1% Formic Acid in
CAN; Flow Rate: 0.800 mL/min; Column Temperature: 40° C.; Injection Volume: 5 mL;
UV: monitor 215, 230, 254, 280, and 300 nm; Purity is reported at 254 nm unless
otherwise noted
Gradient Table:

| | |
|---|---|
| Time (min) | % B |
| 0 | 5 |
| 2.5 | 95 |
| 4.0 | 95 |
| 4.1 | 5 |
| 5.5 | 5 |

^{b}MS Conditions: Instrument: Agilent MSD; Ionization Mode: API-ES;
Gas Temperature: 350° C.; Drying Gas: 11.0 L/min.; Nebulizer Pressure: 55psig;
Polarity: 50% positive, 50% negative; VCap: 3000 V (positive), 2500 V (negative);
Fragmentor: 80 (positive), 120 (negative); Mass Range: 100-1000 m/z; Threshold:
150; Step size: 0.15; Gain: 1; Peak width: 0.15 min.

### General Procedure (Procedure A) for the Suzuki Reaction with 9-(1-Benzyl-piperidin-4-yl)-2-chloro-6-morpholin-4-yl-9H-purine

To a microwave processing tube dimethoxyethane (10 mL), 2M aqueous Na₂CO₃ solution or saturated aqueous NaHCO₃ (2 eq), (Ph₃P)₄Pd (233 mg, 0.2 mmol, 0.05 eq), appropriate boronic acid (1.2 eq) and 9-(1-Benzyl-piperidin-4-yl)-2-chloro-6-morpholin-4-yl-9H-purine (1 eq) are added and the vessel sealed. The mixture is heated to 175° C. for 10 to 20 minutes. The solvents are distilled on a rotary evaporator and the crude compound is purified by preparative HPLC (high pressure liquid chromatography) using ACN/water/NH₃-gradients as eluent or column chromatography with CH₂Cl₂/MeOH/NH₃ to give the product (in 25-65% yield)

### Compound 31: Preparation of 5-[9-(1-benzylpiperidin-4-yl)-6-morpholin-4-yl-9H-purin-2-yl]pyrimidin-2-amine

5-[9-(1-benzylpiperidin-4-yl)-6-morpholin-4-yl-9H-purin-2-yl]pyrimidin-2-amine is prepared from 9-(1-Benzyl-piperidin-4-yl)-2-chloro-6-morpholin-4-yl-9H-purine (100 mg, 0.24 mmol, 1 eq), saturated aqueous NaHCO₃ solution (1 ml), (Ph₃P)₄Pd (25 mg, 0.01 mmol, 0.05 eq), and 2-aminopyrimidine boronic acid (51 mg, 0.363 mmol, 1.5 eq) according to procedure A giving title product (57 mg, 48% yield; mp 238-240° C.; MS (ESI) m/z 472.4).

### Compound 32: Preparation of 5-[9-(1-benzylpiperidin-4-yl)-6-morpholin-4-yl-9H-purin-2-yl]pyridin-2-amine

5-[9-(1-benzylpiperidin-4-yl)-6-morpholin-4-yl-9H-purin-2-yl]pyridin-2-amine is prepared from 9-(1-benzyl-piperidin-4-yl)-2-chloro-6-morpholin-4-yl-9H-purine (100 mg, 0.24 mmol, 1 eq), saturated aqueous NaHCO₃ solution (1 ml), (Ph₃P)₄Pd (25 mg, 0.01 mmol, 0.05 eq), and 5-(4,4,5,5-Tetramethyl-[1,3,2]dioxaborolan-2-yl)-pyridin-2-ylamine (80 mg, 0.363 mmol, 1.5 eq) according to procedure A giving the title product (57 mg, 50% yield; mp 198-200° C.; MS (ESI) m/z 471.5; MS (ESI) m/z 256.8.

### Compound 33: {3-[9-(1-benzylpiperidin-4-yl)-6-morpholin-4-yl-9H-purin-2-yl]phenyl}methanol

5-[9-(1-benzylpiperidin-4-yl)-6-morpholin-4-yl-9H-purin-2-yl]pyridin-2-amine is prepared from 9-(1-benzyl-piperidin-4-yl)-2-chloro-6-morpholin-4-yl-9H-purine (230 mg, 0.55 mmol, 1 eq), saturated aqueous NaHCO₃ solution (2 ml), (Ph₃P)₄Pd (35 mg, 0.03 mmol, 0.05 eq), and 3-hydroxymethyl phenyl boronic acid (122 mg, 0.836 mmol, 1.5 eq) according to procedure A giving the title product (105 mg, 39% yield; mp 172-174° C.; MS (ESI) m/z 485.4; MS (ESI) m/z 263.7; HRMS: calcd. for C₂₈H₃₂N₆O₂+H⁺, 485.26595; found (ESI-FTMS, [M+H]1⁺ 485.26699).

### Compound 34: Preparation of 5-[9-(1-benzylpiperidin-4-yl)-6-morpholin-4-yl-9H-purin-2-yl]nicotinaldehyde

5-[9-(1-benzylpiperidin-4-yl)-6-morpholin-4-yl-9H-purin-2-yl]nicotinaldehyde is prepared from 9-(1-benzyl-piperidin-4-yl)-2-chloro-6-morpholin-4-yl-9H-purine (350 mg, 0.85 mmol, 1 eq), 2M aqueous Na₂CO₃ solution (0.85 mL, 1.7 mmol, 2 eq), (Ph₃P)₄Pd (50 mg, 0.04 mmol, 0.05 eq), 5-(4,4,5,5-Tetramethyl-[1,3,2]dioxaborolan-2-yl)-pyridine-3-carbaldehyde (396 mg, 1.7 mmol, 2 eq) according to procedure A giving the title product (350 mg, 80% yield; MS (ESI) m/z=484.4).

### Compound 35: Preparation of {5-[9-(1-benzylpiperidin-4-yl)-6-morpholin-4-yl-9H-purin-2-yl]pyridin-3-yl}methanol

In a nitrogen flushed 50 mL round bottom flask is charged to a stirring solution of 5-[9-(1-benzylpiperidin-4-yl)-6-morpholin-4-yl-9H-purin-2-yl]nicotinaldehyde (250 mg, 0.52 mmol, 1 eq) in methanol (20 mL), NaBH₄ (39 mg, 1.03 mmol, 2 eq) is added. The mixture is stirred for 30 minutes at room temperature to complete reduction. The solvent is evaporated and the residue dissolved in DMSO and purified by preparative HPLC giving the title product (35 mg, 14% yield; MS (ESI) m/z 486.3).

### Compound 36: Preparation of 5-(6-morpholin-4-yl-9-piperidin-4-yl-9H-purin-2-yl)pyridin-3-ol

In a 25 mL round bottom flask equipped with spin bar and reflux condenser is dissolved 4-[2-(5-Methoxymethoxy-pyridin-3-yl)-6-morpholin-4-yl-purin-9-yl]-piperidine-1-carboxylic acid tert-butyl ester (345 mg, 0.65 mmol) in methanol (5 mL) and conc. HCl (1 mL) is added. The mixture is heated to reflux for 1 h and then stirred for 1 h at room temperature. Precipitation of a white solid, which is collected by filtration, gives the product as the bis-HCl salt (195 mg in 66% yield; MS (ESI) m/z 382.3).

### Procedure B: For the N-alkylation of 5-(6-morpholin-4-yl-9-piperidin-4-yl-9H-purin-2-yl)pyridin-3-ol

To a stirred mixture of 5-(6-morpholin-4-yl-9-piperidin-4-yl-9H-purin-2-yl)pyridin-3-ol (21 mg, 0.06 mmol), the appropriate aldehyde (0.12 mmol, 2 eq) NaBH₃CN (30 mg, 0.47 mmol, 8 eq) in methanol, ZnCl₂ (30 mg, 0.22 mmol, 3.6 eq) in methanol (1 mL) is added. The reaction is stirred for 12 h, then DMSO (1 mL) is added. The mixture is filtered and purified by preparative HPLC (high pressure liquid chromatography) using ACN/water/NH₃-gradients as eluent, to obtain the product after removal of the solvent in 46-66% yield.

### Compound 37: Preparation of 5-(9-{1-[(5-methyl-2-thienyl)methyl]piperidin-4-yl}-6-morpholin-4-yl-9H-purin-2-yl)pyridin-3-ol

5-(6-morpholin-4-yl-9-piperidin-4-yl-9H-purin-2-yl)pyridin-3-ol (22 mg, 0.06 mmol) is N-benzylated according to procedure B above using 5-methylthiophene carbaldehyde to give the title product (16 mg, 49% yield; MS (ESI) m/z 492.6).

### Compound 38: Preparation of 5-{9-[1-(4-chlorobenzyl)piperidin-4-yl]-6-morpholin-4-yl-9H-purin-2-yl}pyridin-3-ol

5-(6-morpholin-4-yl-9-piperidin-4-yl-9H-purin-2-yl)pyridin-3-ol (22 mg, 0.06 mmol) is N-benzylated according to procedure B above using 4-chlorobenzaldehyde giving the title product (19 mg, 56% yield; MS (ESI) m/z 507.1).

### Compound 39: Preparation of 5-{6-morpholin-4-yl-9-[1-(1H-pyrrol-2-ylmethyl)piperidin-4-yl]-9H-purin-2-yl}pyridin-3-ol

5-(6-morpholin-4-yl-9-piperidin-4-yl-9H-purin-2-yl)pyridin-3-ol (22 mg, 0.06 mmol) is N-benzylated according to procedure B above using 2-pyrrolcarboxaldehyde giving the title product (14 mg, 46% yield; MS (ESI) m/z 461.3).

### Compound 40: Preparation of 5-{9-[1-(4-methylbenzyl)piperidin-4-yl]-6-morpholin-4-yl-9H-purin-2-yl}pyridin-3-ol

5-(6-morpholin-4-yl-9-piperidin-4-yl-9H-purin-2-yl)pyridin-3-ol (22 mg, 0.06 mmol) is N-benzylated according to procedure B above using p-tolualdehyde giving the title product (21 mg, 66% yield; MS (ESI) m/z 487.3).

### Compound 41: Preparation of 5-{9-[1-(6-fluoro-pyridin-3-ylmethyl)-piperidin-4-yl]-6-morpholin-4-yl-9H-purin-2-yl}-pyridin-3-ol

5-(6-morpholin-4-yl-9-piperidin-4-yl-9H-purin-2-yl)pyridin-3-ol (200 mg, 0.52 mmol) is N-benzylated according to procedure B above using 6-fluoronicotincarbaldehyde giving the title product (32 mg, 13% yield; MS (ESI) m/z 491.4).

### Compound 42: Preparation of 2-(5-methoxypyridin-3-yl)-6-morpholin-4-yl-9H-purine

To a microwave processing tube is charged dimethoxyethane (10 mL), 2M aqueous Na₂CO₃ solution (4.04 mL, 8.08 mmol, 2 eq), (Ph₃P)₄Pd (233 mg, 0.2 mmol, 0.05 eq), 3-methoxy-5-(4,4,5,5-tetramethyl-[1,3,2]dioxaborolan-2-yl)-pyridine (1140 mg, 4.85 mmol, 1.2 eq) and 2-chloro-6-morpholin-4-yl-9H-purine (968 mg, 4.04 mmol, 1 eq), and the vessel sealed. The mixture is heated to 175° C. for 10 minutes, afterwards, the mixture is evaporated to dryness. MeOH (20 mL) and silica-gel (g) are added and the solvent is removed in vacuo to form a silica-gel plug. The mixture is purified by chromatography with a mixture of CH₂Cl₂/MeOH/NH₃ 20:1:0.1 as eluent giving the product as off white solid (600 mg, 48% yield; MS (ESI) m/z=312.2).

### Compound 43: Preparation of 5-(6-morpholin-4-yl-9H-purin-2-yl)pyridin-3-ol

In a sealed tube 2-(5-Methoxy-pyridin-3-yl)-6-morpholin-4-yl-9H-purine (370 mg, 1.18 mmol) in 48% HBr (15 mL) is placed and heated for 12 h to 110° C. The HBr is removed under reduced pressure and the residue neutralized with saturated aqueous NaHCO₃ solution and extracted with THF (5 mL). The solvent is removed and the product purified by preparative HPLC using ACN/water/TFA and ACN/water/NH₃ giving the title product (6 mg, 1.7% yield; MS (ESI) m/z 299.2).

### Compound 44: Synthesis of (3-{6-morpholin-4-yl-9-[1-(pyridin-2-ylmethyl)piperidin-4-yl]-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 3-pyridinecarboxaldehyde (12.12 mg, 0.112 mmol) in methanol is stirred for 24 hours at room temperature. The mixture is then filtered, dissolved in DMSO (1 mL) and purified by chromatography by HPLC giving 7.8 mg of the title product (21.1% yield, MS (ESI) m/z 486.0).

### Compound 45: Synthesis of (3-{9-[1-(2-fluorobenzyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 2-fluoro-benzaldehyde (14.1 mg, 0.114 mmol) in methanol is stirred for 24 hours at room temperature. The mixture is then filtered, dissolved in DMSO (1 mL) and purified by chromatography by HPLC giving 6.5 mg of the title product (17.1% yield, MS (ESI) m/z 503.5).

### Compound 46: Synthesis of (3-{9-[1-(4-fluorobenzyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and p-fluoro-benzaldehyde (14.16 mg, 0.114 mmol) in methanol is stirred for 24 hours at room temperature. The mixture is then filtered, dissolved in DMSO (1 mL) and purified by chromatography by HLPC giving 6.2 mg of the product (16.2% yield, MS (ESI) m/z 503.5).

### Compound 47: Synthesis of (3-{6-morpholin-4-yl-9-[1-(4-pyridin-4-ylbenzyl)piperidin-4-yl]-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 4-pyridin-4-yl-benzaldehyde (20.9 mg, 0.114 mmol) in methanol is stirred for 24 hours at room temperature. The mixture is then filtered, dissolved in DMSO (1 mL) and purified by chromatography by HPLC giving 7.3 mg of the product (17.1% yield, MS (ESI) m/z 562.6).

### Compound 48: Synthesis of {3-[9-(1-butylpiperidin-4-yl)-6-morpholin-4-yl-9H-purin-2-yl]phenyl}methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and pentanal (8.22 mg, 0.114 mmol) in methanol is stirred for 24 hours at room temperature. The mixture is then filtered, dissolved in DMSO (1 mL) and purified by chromatography by HPLC giving 11 mg of the title product (32.2% yield, MS (ESI) m/z 451.5).

### Compound 49: Synthesis of (3-{9-[1-(2,4-difluorobenzyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 2,4-difluoro-benzaldehyde (16 mg, 0.114 mmol) in methanol is stirred for 24 hours at room temperature. The mixture is then filtered, dissolved in DMSO (1 mL) and purified by chromatography by HPLC giving 7.3 mg of the product (18.1% yield, MS (ESI) m/z 521.5).

### Compound 50: Synthesis of (3-{9-[1-(3-fluorobenzyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 3-fluoro-benzaldehyde (14.1 mg, 0.114 mmol) in methanol is stirred for 24 hours at room temperature. The mixture is then filtered, dissolved in DMSO (1 mL) and purified by chromatography by Gilson giving 5.3 mg of the product (13.8% yield, MS (ESI) m/z 503.5).

### Compound 51: Synthesis of {3-[9-(1-benzylpiperidin-4-yl)-6-morpholin-4-yl-9H-purin-2-yl]phenyl}methanol

A mixture of [3-(6-morpholin-4-yl-9-piperidin-4-yl-9H-purin-2-yl)phenyl]methanol, (30 mg, 0.076 mmol), NaCNBH₃ (25 mg, 0.40 mmol), zinc chloride (20 mg, 0.183 mmol) and 2-fluoro-benzaldehyde (12.1 mg, 0.114 mmol) in methanol is stirred for 24 hours at room temperature. The mixture is then filtered, dissolved in DMSO (1 mL) and purified by chromatography by HPLC giving 5.1 mg of the product (14.2% yield, MS (ESI) m/z 485.6).

### Compound 52: (3-{9-[1-(2-furylmethyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 2-fluoro-benzaldehyde (10.9 mg, 0.114 mmol) in methanol is stirred for 24 hours at room temperature. The mixture is then filtered, dissolved in DMSO (1 mL) and purified by chromatography by HPLC giving 11.2 mg of the product (31% yield, MS (ESI) m/z 475.5).

### Compound 56: Synthesis of tert-butyl 4-(2-choro-6-morpholino-9H-purin-9-yl)piperidine-1-carboxylate

A mixture of tert-butyl 4-(2-choro-6-morpholino-9H-purin-9-yl)piperidine-1-carboxylate (0.35 g, 1.73 mmol) THF (20 mL), triphenyphosphine (0.44 g, 1.7 mmols) is stirred at room temperature for 5 minutes under nitrogen atmosphere. Diethyl azodicarboxylate (0.72 g, 4.1 mmol) is then added to the mixture and stirred for 1 hour. To the mixture is added 2,6-dichloro-purine (0.27 g, 1.0 mmol) and further stirred for 24 hours. The mixture is then taken up in chloroform (200 mL) and water (200 mL). The organic layer is separated, and the water layer extracted twice with chloroform. The combined organic layer is the dried over anhydrous MgSO₄ and filtered. The solvent is evaporated and the residue purified by chromatography on silica using 1:1 Hexanes/EtOAc to give 0.24 g (51%) of the product, Tert-butyl 4-(2-choro-6-morpholino-9H-purin-9-yl)piperidine-1-carboxylate yield (MS (ESI) m/z 423.3).

### Compound 57: Synthesis of tert-butyl 4-{2-(3-hydroxyphenyl)-6-morpholino-9H-purin-9-yl}piperidine-1-carboxylate

A mixture of tert-butyl 4-(2-choro-6-morpholino-9H-purin-9-yl)piperidine-1-carboxylate (0.40 g 0.94 mmol), DMF (2 mL), 3-hydroxyphenylboronic acid (0.196, 1.42 mmol), Pd(Ph₃P)₄ (catalytic amount), sodium carbonate solution (2M) (1.0 ml) is heated to 160° C. for 10 minutes under microwave conditions. The mixture is then filtered through Celite and extracted with chloroform. The organic layer is combined, dried with magnesium sulfate and filtered. The solvent is evaporated and the residue purified by chromatography on silica gel using 2/1 hexanes/EtOAc giving 40 mg (8% yield) of the title product (MS (ESI) m/z 481.3).

### Compound 58: Synthesis of tert-butyl-4-{2-[5-(methoxymethoxy)pyridin-3-yl]-6-morpholin-4-yl-9H-purin-9-yl}piperidine-1-carboxylate

A mixture of tert-butyl 4-(2-choro-6-morpholino-9H-purin-9-yl)piperidine-1-carboxylate (0.355 g 0.84 mmol), DME (8 mL), 3-Methoxymethoxy-5-(4,4,5,5-tetramethyl-[1,3,2]dioxaborolan-2-yl)-pyridine (0.435 g, 1.68 mmol, 2 eq), Pd(Ph₃P)₄ (90 mg, 0.08 mmol, 0.1 eq) (catalytic amount), sodium carbonate solution (2M) (0.84 ml, 2 eq) is heated at 175° C. for 15 minutes in a microwave apparatus. The mixture is then filtered through Celite, taken up in chloroform (150 mL), and water (150 mL), the organic layer separated, extracted with chloroform (150 mL), and washed with water twice. The organic layers are combined, dried with magnesium sulfate, and then filtered. The solvent is evaporated and the residue purified by chromatography on silica gel with as eluent CH₂Cl₂, MeOH, NH₃ to give 0.345 g (78% yield) of the titled product (MS (ESI) m/z 526.4).

### Compound 59: Synthesis of tert-butyl 4-{2-[3-(hydroxymethyl)phenyl]-6-morpholin-4-yl-9H-purin-9-yl}piperidine-1-carboxylate

A mixture of tert-butyl 4-(2-choro-6-morpholino-9H-purin-9-yl)piperidine-1-carboxylate (0.71 g 1.68 mmol), DME (25 mL), 3-hydroxyphenylmethyl boronic acid (0.76 g, 5.0 mmol), Pd(Ph₃P)₄ (catalytic amount), sodium carbonate solution (2M) (1.0 ml) is heated to reflux for 16 hours. The mixture is then filtered through Celite, taken up in chloroform (150 mL) and water (150 mL), and the organic layer separated, extracted with chloroform (150 mL), and washed with water twice. The organic layer is combined, dried with magnesium sulfate, and then filtered. The solvent is evaporated and the residue purified by chromatography on silica gel 2/1 hexanes/EtOAc to give 0.80 g (89% yield) of the title product (MS (ESI) m/z 495.4).

### Compound 60: Preparation of (3-{6-morpholin-4-yl-9-[1-(2,4,6-trifluorobenzyl)piperidin-4-yl]-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 2,4,6-trifluoro-benzaldehyde (18 mg, 0.11 mmol) in methanol is stirred for 24 hours at room temperature. The mixture is then filtered, dissolved in DMSO (1 mL) and purified by chromatography by HPLC to give 12 mg (30% yield) of the title product (MS (ESI) m/z 539.6).

### Compound 61: Synthesis of [3-(9-{1-[(6-fluoropyridin-3-yl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 6-fluoronicotinicaldehyde (14 mg, 0.11 mmol) in methanol is stirred for 24 hours at room temperature. The mixture is then filtered, dissolved in DMSO (1 mL) and purified by chromatography by HPLC to give 9 mg (24% yield) of the title product.

### Compound 62: Synthesis of (3-{9-[1-(3,4-difluorobenzyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 3,4-difluoro-benzaldehyde (16 mg, 0.11 mmol) in methanol is stirred for 24 hours at room temperature. The mixture is then filtered, dissolved in DMSO (1 mL) and purified by chromatography by HPLC to give 10 mg (26% yield) of the title product (MS (ESI) m/z 521=7).

### Compound 63: [3-(9-{1-[(6-chloropyridin-3-yl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 6-chloronicotinoylaldehyde (16 mg, 0.11 mmol) in methanol is stirred for 24 hours at room temperature. The mixture is then filtered, dissolved in DMSO (1 mL) and purified by chromatography by HPLC to give 10 mg (26% yield) of the title product. MS (ESI) m/z 521.

### Compound 64: Synthesis of [3-(9-{1-[(6-methoxypyridin-3-yl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 6-methoxynicotinoylaldehyde (16 mg, 0.11 mmol) in methanol is stirred for 24 hours at room temperature. The mixture is then filtered, dissolved in DMSO (1 mL) and purified by chromatography by HPLC to give 10 mg (26% yield) of the title product (MS (ESI) m/z 516.6).

### Compound 65: [3-(9-{1-[(2,6-dimethoxypyridin-3-yl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 3,5-dimethoxy-4-pyridinecarbaldehyde (62 mg, 0.37 mmol) in methanol is stirred for 24 hours at room temperature. The mixture is then filtered, dissolved in DMSO (1 mL) and purified by chromatography by HPLC to give 12 mg (29% yield) of the title product (MS (ESI) m/z 546.6).

### Compound 66: [3-(9-{1-[(5-fluoropyridin-3-yl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 5-fluoronicotinoylaldehyde (14 mg, 0.11 mmol) in methanol is stirred for 24 hours at room temperature. The mixture is then filtered, dissolved in DMSO (1 mL) and purified by chromatography by HPLC to give 10 mg (25% yield) of the title product (MS (ESI) m/z 504.6).

### Compound 67: Synthesis of [3-(9-{1-[(5-methyl-2-thienyl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 5-methyl-2-thiphenecarbaldehyde (14 mg, 0.11 mmol) in methanol is stirred for 24 hours at room temperature. The mixture is then filtered, dissolved in DMSO (1 mL) and purified by chromatography by HPLC to give 12 mg (31% yield) of the title product (MS (ESI) m/z 505.7).

### Compound 68: Synthesis of (3-{6-morpholin-4-yl-9-[1-(1H-pyrrol-2-ylmethyl)piperidin-4-yl]-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and pyrrole-2-carbaldehyde (13 mg, 0.11 mmol) in methanol is stirred for 24 hours at room temperature. The mixture is then filtered, dissolved in DMSO (1 mL) and purified by chromatography by HPLC to give 4 mg (10% yield) of the title product (MS (ESI) m/z 474.4).

### Compound 69: Synthesis of (3-{9-[1-(2-chloro-4-fluorobenzyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 2-chloro-4-fluorobenzaldehyde (11 mg, 0.11 mmol) in methanol is stirred for 24 hours at room temperature. The mixture is then filtered, dissolved in DMSO (1 mL) and purified by chromatography by HPLC to give 6 mg (16% yield) of the product. MS (ESI) m/z 538.2.

### Compound 70: Synthesis of (3-{9-[1-(1H-imidazol-2-ylmethyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and imidazolo-2-carbaldehyde (15 mg, 0.11 mmol) in methanol is stirred for 24 hours at room temperature. The mixture is then filtered, dissolved in DMSO (1 mL) and purified by chromatography by HPLC to give 15 mg (41% yield) of the title product (MS (ESI) m/z=475.4).

### Compound 71: Synthesis of [3-(9-{1-[(6-bromopyridin-2-yl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 6-bromopicolinicaldehyde (22 mg, 0.11 mmol) in methanol is stirred for 24 hours at room temperature. The mixture is then filtered, dissolved in DMSO (1 mL) and purified by chromatography by HPLC to give 8 mg (20% yield) of the title product (MS (ESI) m/z 565.5).

### Compound 72: Synthesis of [3-(6-morpholin-4-yl-9-{1-[(6-morpholin-4-ylpyridin-2-yl)methyl]piperidin-4-yl}-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 6-morpholinoylpicolinicaldehyde (15 mg, 0.11 mmol) in methanol is stirred for 24 hours at room temperature. The mixture is then filtered, dissolved in DMSO (1 mL) and purified by chromatography by HPLC to give 5 mg (11% yield) of the title product (MS (ESI) m/z 571.7).

### Compound 73: Synthesis of [3-(9-{1-[(5-fluoro-1H-indol-3-yl)methyl]piperidin-4-yl}-6-morpholin-4-yl-9H-purin-2-yl)phenyl]methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 5-fluoroindole-3-carbaldehyde (16 mg, 0.11 mmol) in methanol is stirred for 24 hours at room temperature. The mixture is then filtered, dissolved in DMSO (1 mL) and purified by chromatography by HPLC to give 13 mg (30% yield) of the title product (MS (ESI) m/z 542.7).

### Compound 74: Synthesis of (3-{9-[1-(5,6-dihydro-8H-imidazo[2,1-c][1,4]oxazin-3-ylmethyl)piperidin-4-yl]-6-morpholin-4-yl-9H-purin-2-yl}phenyl)methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 5,6-Dihydro-8H-imidazo[2,1-c][1,4]oxazine-3-carbaldehyde (17 mg, 0.11 mmol) in methanol is stirred for 24 hours at room temperature. The mixture is then filtered, dissolved in DMSO (1 mL) and purified by chromatography by HPLC to give 17 mg (43% yield) of the title product (MS (ESI) m/z 531.5).

### Compound 75: Synthesis of {3-[9-(1-{4-[3-dimethylamino)propoxy]benzyl}piperidin-4-yl)-6-morpholin-4-yl-9H-purin-2-yl]phenyl}methanol

A mixture of [3-(6-morpholin-4-yl-9-piperidin-4-yl-9H-purin-2-yl)phenyl]methanol (30 mg, 0.076 mmol), NaCNBH₃ (25 mg, 0.40 mmol), zinc chloride (20 mg, 0.183 mmol) and 4-N,N-dimethylpropoxybenzadlehyde (23 mg, 0.11 mmol) in methanol is stirred for 24 hours at room temperature. The mixture is then filtered, dissolved in DMSO (1 mL) and purified by chromatography by HPLC to give 10 mg (23% yield) of the title product (MS (ESI) m/z 586.8).

### Compound 76: Synthesis of 3-{6-morpholin-4-yl-9-[1-(pyridin-3-ylmethyl)piperidin-4-yl]-9H-purin-2-yl}phenol

A mixture of [3-(6-morpholin-4-yl-9-piperidin-4-yl-9H-purin-2-yl)phenol (50 mg, 0.131 mmol), NaCNBH₄ (25 mg, 0.40 mmol), zinc chloride (20 mg, 0.183 mmol) and 3-pyridinecarbaldehyde (28 mg, 0.262 mmol) in methanol is stirred for 24 hours at room temperature. The mixture is then filtered, dissolved in DMSO (1 mL) and purified by chromatography by HPLC to give 40 mg (65% yield) of the title product (MS (ESI) m/z 472.4).

### Compound 77: Synthesis of 3-{6-morpholin-4-yl-9-[1-(pyridin-2-ylmethyl)piperidin-4-yl]-9H-purin-2-yl}phenol

A mixture of [3-(6-morpholin-4-yl-9-piperidin-4-yl-9H-purin-2-yl)phenol (50 mg, 0.131 mmol), NaCNBH₃ (25 mg, 0.40 mmol), zinc chloride (20 mg, 0.183 mmol) and 2-pyridinecarbaldehyde (28 mg, 0.262 mmol) in methanol is stirred for 24 hours at room temperature. The mixture is then filtered, dissolved in DMSO (1 mL) and purified by chromatography by HPLC to give 43 mg (70% yield) of the title product (MS (ESI) m/z 472.3).

### Compound 78: Synthesis of 3-(9-{1-[(6-chloropyridin-3-yl)methyl]piperidin-4-yl}-6-morpholin-4-yl-9H-purin-2-yl)phenol

A mixture of [3-(6-morpholin-4-yl-9-piperidin-4-yl-9H-purin-2-yl)phenol (50 mg, 0.131 mmol), NaCNBH₃ (25 mg, 0.40 mmol), zinc chloride (20 mg, 0.183 mmol) and 6-chloronicotinoylaldehyde (37 mg, 0.262 mmol) in methanol is stirred for 24 hours at room temperature. The mixture is then filtered, dissolved in DMSO (1 mL) and purified by chromatography by HPLC to give 32 mg (48% yield) of the title product (MS (ESI) m/z 507.2).

### Compound 79: Synthesis of 3-(9-{1-[(6-methoxypyridin-3-yl)methyl]piperidin-4-yl}-6-morpholin-4-yl-9H-purin-2-yl)phenol

A mixture of [3-(6-morpholin-4-yl-9-piperidin-4-yl-9H-purin-2-yl)phenol (50 mg, 0.131 mmol), NaCNBH₃ (25 mg, 0.40 mmol), zinc chloride (20 mg, 0.183 mmol) and 6-methoxynicotinoylaldehyde (36 mg, 0.262 mmol) in methanol is stirred for 24 hours at room temperature. The mixture is then filtered, dissolved in DMSO (1 mL) and purified by chromatography by HPLC to give 32 mg (49% yield) of the title product (MS (ESI) m/z 502.7).

### Compound 80: Synthesis of 3-(9-{1-[(2,6-dimethoxypyridin-4-yl)methyl]piperidin-4-yl}-6-morpholin-4-yl-9H-purin-2-yl)phenol

A mixture of [3-(6-morpholin-4-yl-9-piperidin-4-yl-9H-purin-2-yl)phenol (50 mg, 0.131 mmol), NaCNBH₃ (25 mg, 0.40 mmol), zinc chloride (20 mg, 0.183 mmol) and 3,5-dimethoxy-4-pyridinecarbaldehyde (44 mg, 0.262 mmol) in methanol is stirred for 24 hours at room temperature. The mixture is then filtered, dissolved in DMSO (1 mL) and purified by chromatography by HPLC to give 41 mg (59% yield) of the title product (MS (ESI) m/z 532.7).

### Compound 81: Synthesis of 3-(9-{1-[(6-bromopyridin-3-yl)methyl]piperidin-4-yl}-6-morpholin-4-yl-9H-purin-2-yl)phenol

A mixture of [3-(6-morpholin-4-yl-9-piperidin-4-yl-9H-purin-2-yl)phenol (50 mg, 0.131 mmol), NaCNBH₃ (25 mg, 0.40 mmol), zinc chloride (20 mg, 0.183 mmol) and 6-bromonicotinoylaldehyde (49 mg, 0.262 mmol) in methanol is stirred for 24 hours at room temperature. The mixture is then filtered, dissolved in DMSO (1 mL) and purified by chromatography by HPLC to give 21 mg (29% yield) of the title product (MS (ESI) m/z 551.7).

### Compound 82: Synthesis of 3-(6-morpholin-4-yl-9-{1-[(6-morpholin-4-ylpyridin-3-yl)methyl]piperidin-4-yl}-9H-purin-2-yl)phenol

A mixture of [3-(6-morpholin-4-yl-9-piperidin-4-yl-9H-purin-2-yl)phenol (50 mg, 0.131 mmol), NaCNBH₃ (25 mg, 0.40 mmol), zinc chloride (20 mg, 0.183 mmol) and 6-morpholinoylnicotinoylaldehyde (50 mg, 0.262 mmol) in methanol is stirred for 24 hours at room temperature. The mixture is then filtered, dissolved in DMSO (1 mL) and purified by chromatography by HPLC to give 33 mg (45% yield) of the title product (MS (ESI) m/z 557.7).

### Compound 83: Synthesis of 3-[9-(1-{[4-(dimethylamino)-1-naphthyl]methyl}piperidin-4-yl)-6-morpholin-4-yl-9H-purin-2-yl]phenol

A mixture of [3-(6-morpholin-4-yl-9-piperidin-4-yl-9H-purin-2-yl)phenol (50 mg, 0.131 mmol), NaCNBH₃ (25 mg, 0.40 mmol), zinc chloride (20 mg, 0.183 mmol) and 4-dimethylamino-naphthalene-1-carbaldehyde (52 mg, 0.262 mmol) in methanol is stirred for 24 hours at room temperature. The mixture is then filtered, dissolved in DMSO (1 mL) and purified by chromatography by HPLC to give 32 mg (43% yield) of the title product (MS (ESI) m/z 564.8).

### Compound 84: Synthesis of 3-(9-{1-[(6-fluoropyridin-3-yl)methyl]piperidin-4-yl}-6-morpholin-4-yl-9H-purin-2-yl)phenol

A mixture of [3-(6-morpholin-4-yl-9-piperidin-4-yl-9H-purin-2-yl)phenol (100 mg, 0.262 mmol), NaCNBH₃ (50 mg, 0.80 mmol), zinc chloride (40 mg, 0.36 mmol) and 4-6-fluoronicotinic aldehyde (66 mg, 0.522 mmol) in methanol is stirred for 24 hours at room temperature. The mixture is then filtered, dissolved in DMSO (2 mL) and purified by chromatography by HPLC to give 68 mg (53% yield) of the title product (MS (ESI)=m/z 490.4).

### 2-Chloro-6-morpholin-4-yl-9-(2-piperidin-1-yl-ethyl)-9H-purine

To 2-chloro-6-morpholinoylpurine (500 mg, 2.09 mmol) 1-hydroxyethylpiperidine (405 mg, 3.13 mmol) and PBu₃ (821 mg, 3.13 mmol) dissolved in THF (2 mL) was added DEAD (545 mg, 3.13 mmol). The mixture is stirred over night, and the crude product purified via silica gel chromatography (10% MeOH/EtOAc) to afford 330 mg (45% yield) of a yellow solid.

### Compound 85: Synthesis of 3-[6-morpholin-4-yl-9-(2-piperidin-1-ylethyl)-9H-purin-2-yl]phenol

3-[6-morpholin-4-yl-9-(2-piperidin-1-ylethyl)-9H-purin-2-yl]phenol is prepared from 2-Chloro-6-morpholin-4-yl-9-(2-piperidin-1-yl-ethyl)-9H-purine (100 mg, 0.29 mmol, 1 eq), saturated aqueous NaHCO₃ (1 ml), (Ph₃P)₄Pd (18 mg, 0.02 mmol, 0.05 eq), and 3-hydroxymethyl phenyl boronic acid (59 mg, 0.428 mmol, 1.5 eq) in DME (2 mL) by heating in a microwave apparatus to 175° C. for 15 min. The solvent is removed and the material dissolved in DMSO (2 mL) and purified by HPLC to give the title product (45 mg, 39% yield; MS (ESI) m/z 409.4).

### Compound 86: Synthesis of {3-[6-morpholin-4-yl-9-(2-piperidin-1-ylethyl)-9H-purin-2-yl]phenyl}methanol

{3-[6-morpholin-4-yl-9-(2-piperidin-1-ylethyl)-9H-purin-2-yl]phenyl}methanol was prepared from 2-Chloro-6-morpholin-4-yl-9-(2-piperidin-1-yl-ethyl)-9H-purine (130 mg, 0.37 mmol, 1 eq), saturated aqueous NaHCO₃ solution (0.37 ml), (Ph₃P)₄Pd (24 mg, 0.02 mmol, 0.05 eq), and 3-hydroxymethyl phenyl boronic acid (85 mg, 0.556 mmol, 1.5 eq) in DMF (1.5 mL) by heating in a microwave apparatus to 175° C. for 15 min. The solvent is removed and the material dissolved in DMSO (2 mL) and purified by HPLC to give the product (109 mg, 70% yield; MS (ESI) m/z 423.4).

### Compound 87: Synthesis of 5-[9-(1-benzylpiperidin-4-yl)-6-morpholin-4-yl-9H-purin-2-yl]pyridin-3-ol

5-(6-morpholin-4-yl-9-piperidin-4-yl-9H-purin-2-yl)pyridin-3-ol (100 mg, 0.26 mmol) was N-benzylated according to procedure B above using benzaldehyde to give the titled product (40 mg, yield, 32%; MS (ESI) m/z 472.4).

### Compound 88: Synthesis of 5-(9-{1-[(6-fluoropyridin-3-yl)methyl]piperidin-4-yl}-6-morpholin-4-yl-9H-purin-2-yl)pyridin-3-ol

5-(6-morpholin-4-yl-9-piperidin-4-yl-9H-purin-2-yl)pyridin-3-ol (200 mg, 0.52 mmol) was N-benzylated according to procedure B above using 6-fluoro nicotinyl carbaldehyde to give the titled product (23 mg, yield, 9%; MS (ESI) m/z 491.4).

### Compound 89: Synthesis of 1-methyl-3-(4-(6-morpholino-9-(1-(pyridin-3-ylmethyl)piperidin-4-yl)-9H-purin-2-yl)phenyl)urea

Step 1: A mixture of 2,6-dichloropurine (0.47 g, 2.5 mmol), tert-butyl 4-hydroxypiperidine-1-carboxylate (1.0 g, 5.0 mmol) and triphenylphosphine (1.3 g, 5.0 mmol) in tetrahydrofuran (20 mL) was cooled to 0° C. Diisopropylazodicarboxylate (1 mL) was added to the mixture, which was allowed to warm to room temperature. The mixture was concentrated to dryness under reduced pressure. The residue was purified by reverse phase HPLC, employing a gradient elution of 80% A solvent (0.1% aqueous trifluoroacetic acid) to 100% B solvent (acetonitrile). Tert-butyl 4-(2,6-dichloro-9H-purin-9-yl)piperidine-1-carboxylate was obtained as a solid (800 mg, 86%).

Step 2: Tert-butyl 4-(2,6-dichloro-9H-purin-9-yl)piperidine-1-carboxylate (0.23 g, 0.62 mmol) was taken up as a suspension in ethanol and treated with morpholine. Mixture heated until solids had dissolved and then concentrated to dryness under reduced pressure to afford tert-butyl 4-(2-chloro-6-morpholino-9H-purin-9-yl)piperidine-1-carboxylate in an assumed quantitative yield. MS (ES⁺): 423.1, 425.1 (M+H)⁺

Step 3: Crude tert-butyl 4-(2-chloro-6-morpholino-9H-purin-9-yl)piperidine-1-carboxylate (approximately 2.2 mmol) was dissolved in dichloromethane (50 mL) and treated with trifluoroacetic acid (5 mL). Mixture was concentrated under reduced pressure and triturated with diethyl ether to give 4-(2-chloro-9-(piperidin-4-yl)-9H-purin-6-yl)morpholine trifluoroacetate (0.61 g, 63%). MS (ES⁺): 323.0, 325.0 (M+H)⁺

Step 4: A suspension of 4-(2-chloro-9-(piperidin-4-yl)-9H-purin-6-yl)morpholine trifluoroacetate (0.30 g, 0.69 mmol) in tetrahydrofuran (15 mL) was treated with pyridine 3-carboxaldehyde (0.11 g, 1.0 mmol), followed after fifteen minutes by sodium triacetoxyborohydride (0.21 g, 1.0 mmol). Upon completion, the mixture was diluted with dichloromethane and washed successively with saturated aqueous sodium hydrogen carbonate solution, and 1 M sodium hydroxide solution. The organic phase was dried over anhydrous magnesium sulfate, filtered, and concentrated under reduced pressure to afford crude 4-(2-chloro-9-(1-(pyridin-3-ylmethyl)piperidin-4-yl)-9H-purin-6-yl)morpholine (0.29 g, 100%). MS (ES⁺): 414.1, 416.1 (M+H)⁺

Step 5: A mixture of crude 4-(2-chloro-9-(1-(pyridin-3-ylmethyl)piperidin-4-yl)-9H-purin-6-yl)morpholine (90 mg, 0.22 mmol), tetrakis(triphenylphosphine)palladium (25 mg, 0.02 mmol), and 4-aminophenylboronic acid pinacol ester (71 mg, 0.33 mmol) in 1,2-dimethoxyethane (2 mL) and 2 M aqueous sodium carbonate (0.5 mL) was heated in a microwave reactor for one hour at 180° C. After being allowed to cool to room temperature, the mixture was partitioned between ethyl acetate and water. The aqueous phase was extracted with ethyl acetate. Organics were washed with saturated aqueous sodium chloride solution, dried over anhydrous magnesium sulfate, filtered, and concentrated under reduced pressure to afford crude 4-(6-morpholino-9-(1-(pyridin-3-ylmethyl)piperidin-4-yl)-9H-purin-2-yl)aniline as a brown syrup. MS (ES⁺): 471.1, 472.1 (M+H)⁺

Step 6: Crude 4-(6-morpholino-9-(1-(pyridin-3-ylmethyl)piperidin-4-yl)-9H-purin-2-yl)aniline (100 mg, approx 0.21 mmol) was dissolved in dichloromethane (1 mL) and then treated with triphosgene (32 mg). Additional dichloromethane was added for solubility. After five minutes, methylamine solution (2.0 M in tetrahydrofuran, 2 mL) was added to the suspension. The mixture was concentrated under reduced pressure and purified by reverse phase HPLC, employing a gradient elution of 95% A solvent (0.1% aqueous trifluoroacetic acid) to 90% B solvent (acetonitrile) to afford 1-methyl-3-(4-(6-morpholino-9-(1-(pyridin-3-ylmethyl)piperidin-4-yl)-9H-purin-2-yl)phenyl)urea trifluoroacetate (31 mg). MS (ES⁻): 528.1, 529.1 (M+H)⁺

### Compound 90: Synthesis of 1-ethyl-3-(4-(6-morpholino-9-(1-(pyridin-3-ylmethyl)piperidin-4-yl)-9H-purin-2-yl)phenyl)urea

Crude 4-(6-morpholino-9-(1-(pyridin-3-ylmethyl)piperidin-4-yl)-9H-purin-2-yl)aniline (100 mg, approx 0.21 mmol) was dissolved in dichloromethane (1 mL) and then treated with triphosgene (32 mg). Additional dichloromethane was added for solubility. After five minutes, ethylamine solution (2.0 M in tetrahydrofuran, 2 mL) was added to the suspension. The mixture was concentrated under reduced pressure and purified by reverse phase HPLC, employing a gradient elution of 95% A solvent (0.1% aqueous trifluoroacetic acid) to 90% B solvent (acetonitrile) to afford 1-ethyl-3-(4-(6-morpholino-9-(1-(pyridin-3-ylmethyl)piperidin-4-yl)-9H-purin-2-yl)phenyl)urea trifluoroacetate (24 mg); MS (ES⁻): 542.3 (M+H)⁺

### Compound 91: Synthesis of 1-(2-(3-hydroxyphenyl)-6-morpholino-9H-purin-9-yl)prop-2-en-1-one

Step 1: 2,6-dichloropurine (approximately 0.20 g, 1.1 mmol) was taken up in ethanol (50 mL) and treated with morpholine (2 mL). The white precipitate was collected by filtration, washed with ethanol, and dried under house vacuum to provide 4-(2-chloro-9H-purin-6-yl)morpholine. MS (ES⁺): 240.0, 242.0 (M+H)⁺

Step 2: A mixture of 4-(2-chloro-9H-purin-6-yl)morpholine (0.18 g, 0.75 mmol), tetrakis(triphenylphosphine)palladium (30 mg), and 3-hydroxyphenylboronic acid (0.16 g, 1.1 mmol) in 1,2-dimethoxyethane (2.6 mL) and 2 M aqueous sodium carbonate (0.75 mL) was heated in a microwave reactor for one hour at 180° C. After being allowed to cool to room temperature, the mixture was acidified with 5% aqueous potassium hydrogen sulfate solution and then extracted with ethyl acetate. Organics were washed successively with water and saturated aqueous sodium hydrogen carbonate solution, then dried over anhydrous magnesium sulfate, filtered, and concentrated under reduced pressure to afford a crude white solid. The crude material was purified by reverse phase HPLC, employing a gradient elution of 85% A solvent (0.1% aqueous trifluoroacetic acid) to 100% B solvent (acetonitrile) to afford 3-(6-morpholino-9H-purin-2-yl)phenol as a white powder (80 mg). MS (ES⁺): 298.0 (M+H)⁺

Step 3: 3-(6-morpholino-9H-purin-2-yl)phenol (80 mg, 0.27 mmol) was dissolved in N,N-dimethylacetamide (2 mL) and then treated with acryloyl chloride (200 μL). The crude mixture was purified by reverse phase HPLC, employing a gradient elution of 85% A solvent (0.1% aqueous trifluoroacetic acid) to 100% B solvent (acetonitrile) to afford 1-(2-(3-hydroxyphenyl)-6-morpholino-9H-purin-9-yl)prop-2-en-1-one (20 mg). MS (ES⁺): 352.3 (M+H)⁺

### Biological Evaluation—P13K Fluorescence Polarization Assay Protocol

PI3-Kinase reactions are performed in 5 mM HEPES, pH 7, 2.5 mM MgCl₂, and 25 μM ATP, with diC8-PI(4,5)P2 (Echelon, Salt Lake City Utah) as substrate. Nunc 384 well black polypropylene fluorescent plates are used for PI3K assays. Reactions are quenched by the addition of EDTA to a final concentration of 10 mM. Final reaction volumes are 10 μl. For evaluation of PI3K inhibitors, 5 ng of enzyme and 2.5 μM of substrate are used per 10 μl reaction volume, and inhibitor concentrations range from 100 pM to 20 μM; the final level of DMSO in reactions never exceed 2%. Reactions are allowed to proceed for one hour at 25° C. After 1 hour, GST-tagged GRP1 (general receptor for phosphoinositides) PH domain fusion protein is added to a final concentration of 100 nM, and BODIPY-TMRI(1,3,4,5)P4 (Echelon) is also added to a final concentration of 5 nM. Final sample volumes are 25 μl with a final DMSO concentration of 0.8%. Assay Plates are read on Perkin-Elmer Envision plate readers with appropriate filters for Tamra [BODIPY-TMRI(1,3,4,5)P4].

### Qualitative and Quantitative Cell Based Assays Using Sulforhodamine B (SRB) to Identify Inhibitors of P13 Kinase

Cell Lines used are human pancreatic (PC3) and ovarian (OVCAR3) tumor cell lines. PC3 and OVCAR3 are plated in 96-well culture plates at approximately 3000 cells per well. One day following plating, various concentrations of PI3K inhibitors in DMSO are added to cells (final DMSO concentration in cell assays is 0.25%). Three days after drug treatment, viable cell densities are determined by cell mediated metabolic conversion of the dye MTS, a well-established indicator of cell proliferation in vitro. Cell growth assays are performed using kits purchased from Promega Corporation (Madison, Wis.), following the protocol provided by the vendor. Measuring absorbance at 490 nm generates MTS assay results. Compound effect on cell proliferation is assessed relative to untreated control cell growth. The drug concentration that conferred 50% inhibition of growth is determined as IC₅₀ (μM).

Qualitative screen: To calculate % inhibition of a compound at 25 μM, the following formula is used: 1-(experimental absorbance @ 25 μM compound/“0” control absorbance)×100=% inhibition at 25 μM. Compounds exhibiting >50% inhibition at 25 μM are then placed in the quantitative assay.

Quantitative Assay: A standard curve is constructed by plotting the concentration of compound against the average absorbance calculated at that concentration. A curve is plotted and the concentration at which the curve passes through the 50% absorbance mark seen in the “0” control well is the IC₅₀ calculated for that compound. Roymans, et al., Eur. J. Biochem. 268: 487 (2001); Fruman, et al., Eur. J. Biochem. 67: 481 (1998).

### mTOR Kinase Inhibitor Assay Methods

### mTOR Enzyme Assay

The routine human TOR assays with purified enzyme are performed in 96-well plates by DELFIA format as follows. Enzymes are first diluted in kinase assay buffer (10 mM HEPES (pH 7.4), 50 mM NaCl, 50 mM β-glycerophosphate, 10 mM MnCl₂, 0.5 mM DTT, 0.25 μM microcystin LR, and 100 μg/mL BSA). To each well, 12 μL of the diluted enzyme are mixed briefly with 0.5 μL test inhibitor or control vehicle dimethylsulfoxide (DMSO). The kinase reaction is initiated by adding 12.5 μL kinase assay buffer containing ATP and His6-S6K to give a final reaction volume of 25 μL containing 800 ng/mL FLAG-TOR, 100 μM ATP and 1.25 μM His6-S6K. The reaction plate is incubated for 2 hours (linear at 1-6 hours) at room temperature with gentle shaking and then terminated by adding 25 μL Stop buffer (20 mM HEPES (pH 7.4), 20 mM EDTA, 20 mM EGTA). The DELFIA detection of the phosphorylated (Thr-389) His6-S6K is performed at room temperature using a monoclonal anti-P(T389)-p70S6K antibody (1A5, Cell Signaling) labeled with Europium-N1-ITC (Eu) (10.4 Eu per antibody, PerkinElmer). The DELFIA Assay buffer and Enhancement solution are purchased from PerkinElmer. 45 μL of the terminated kinase reaction mixture is transferred to a MaxiSorp plate (Nunc) containing 55 μL PBS. The His6-S6K is allowed to attach for 2 hours after which the wells are aspirated and washed once with PBS. 100 μL of DELFIA Assay buffer with 40 ng/mL Eu-P(T389)-S6K antibody is added. The antibody binding is continued for 1 hour with gentle agitation. The wells are then aspirated and washed 4 times with PBS containing 0.05% Tween-20 (PBST). 100 μL of DELFIA Enhancement solution is added to each well and the plates read in a PerkinElmer Victor model plate reader.

### In vitro Cell Growth Assay

Cells of human tumor lines LNCap and MDA468 are plated in 96-well culture plates at approximately 3000 cells per well. One day following plating, various doses of mTOR inhibitors are added to cells. Three days after drug treatment, viable cell densities are determined by metabolic conversion (by viable cells) of the dye MTS, a well-established cell proliferation assay. The assays are performed using an assay kit purchased from Promega Corp. (Madison, Wis.) following the protocol supplied with the kit. The MTS assay results are read in a 96-well plate reader by measuring absorbance at 490 nm. The effect of each compound and its concentration is calculated as percent of control growth relative to the vehicle-treated cells grown in the same culture plate. The drug concentration that conferred 50% inhibition of growth is determined as IC₅₀ (μM).

### Rat1-IGF1 Stimulated PI3K/AKT/TOR Activation Assay

For IGF-1 induction experiments, Rat1 cells are plated in 6-well culture plates and serum-starved for 24 hours. Serum-starved cells are treated either with control vehicle or with various concentrations of mTOR inhibitors for 2 hours, stimulated by IGF-1 (100 ng/mL) for 30 minutes. Total cellular lysates are prepared using NuPAGE-LDS sample buffer (Invitrogen), sonicated and then clarified by centrifugation. Equal amounts of proteins are subject to immunoblotting analysis using NuPAGE electrophoresis system and probed with phosphor-specific antibodies against AKT, GSK3, FKHRL, TOR, S6K1, 4EBP1.

### Tumor Cell TOR Inhibition Assay

Human prostate tumor LNCap cells are plated in 6-well plates in growth media for overnight. Cells were treated with various doses of mTOR inhibitors for 6 hours. Total cellular lysates are prepared and analyzed as in Rat1-IGF1 assay.

TABLE 2

| Compound | | PI3 Kinase | TOR Kinase |
|---|---|---|---|
| Number | Compound Name | Median IC₅₀ (nM) | Median IC₅₀ (uM) |
| 1 | 6-morpholin-4-yl-2-(2-thienyl)-9H-purine | 724 | 2.8 |
| 2 | 6-morpholin-4-yl-2-(3-thienyl)-9H-purine | 3290 | 3.9 |
| 3 | 2-(6-morpholin-4-yl-9H-purin-2-yl)phenol | 616 | 2.5 |
| 4 | 4-(6-morpholin-4-yl-9H-purin-2-yl)phenol | 2677 | 0.45 |
| 5 | [4-(6-morpholin-4-yl-9H-purin-2- | 3750 | 4.5 |
| | yl)phenyl]methanol | | |
| 6 | 2-(1H-indol-5-yl)-6-morpholin-4-yl-9H- | 2749 | 0.18 |
| | purine | | |
| 7 | 2-(1,3-benzodioxol-5-yl)-6-morpholin-4-yl- | 1227 | 8.9 |
| | 9H-purine | | |
| 8 | 6-morpholin-4-yl-2-(3-nitrophenyl)-9H- | 8900 | Not determined |
| | purine | | |
| 9 | 2-(1-benzothien-3-yl)-6-morpholin-4-yl- | 1999 | Not determined |
| | 9H-purine | | |
| 10 | 6-morpholin-4-yl-2-[3- | 5712 | Not determined |
| | (trifluoromethyl)phenyl]-9H-purine | | |
| 11 | 2-(3-methylphenyl)-6-morpholin-4-yl-9H- | 4213 | Not determined |
| | purine | | |
| 12 | 2-(3-isopropylphenyl)-6-morpholin-4-yl- | 9922 | Not determined |
| | 9H-purine | | |
| 13 | 3-(6-morpholin-4-yl-9H-purin-2- | 3588 | Not determined |
| | yl)benzonitrile | | |
| 14 | 2-biphenyl-3-yl-6-morpholin-4-yl-9H- | 4443 | Not determined |
| | purine | | |
| 15 | N-(3-(6-morpholino-9H-purin-2- | 3899 | Not determined |
| | yl)phenyl)methanesulfonamide | | |
| 16 | 6-morpholin-4-yl-2-(2-phenoxyphenyl)- | 8935 | Not determined |
| | 9H-purine | | |
| 17 | N,N-dimethyl-4-(6-morpholin-4-yl-9H- | 5928 | Not determined |
| | purin-2-yl)benzamide | | |
| 18 | 2-(2,3-dihydro-1,4-benzodioxin-6-yl)-6- | 3890 | Not determined |
| | morpholin-4-yl-9H-purine | | |
| 19 | 2-(3-furyl)-6-morpholin-4-yl-9H-purine | 4340 | Not determined |
| 20 | 2-[3-(methylsulfonyl)phenyl]-6-morpholin- | 4255 | Not determined |
| | 4-yl-9H-purine | | |
| 21 | 3-(6-Piperidin-1-yl-9H-purin-2-yl)-phenol | 4303 | Not determined |
| 22 | 2-(4-Methanesulfonyl-phenyl)-6- | 2165 | Not determined |
| | morpholin-4-yl-9H-purine | | |
| 23 | 2-[3-(3,5-Dimethoxy-benzyloxy)-phenyl]- | 6198 | 16.5 |
| | 6-morpholin-4-yl-9H-purine | | |
| 24 | 2-(4-Benzyloxy-3-chloro-phenyl)-6- | 4532 | >20.000 |
| | morpholin-4-yl-9H-purine | | |
| 25 | [3-(6-Morpholin-4-yl-9-piperidin-4-yl-9H- | 57 | 7.2 |
| | purin-2-yl)-phenyl]-methanol | | |
| 26 | 1-(4-(2-(3-(hydroxymethyl)phenyl)-6- | 189 | 2.1 |
| | morpholino-9H-purin-9-yl)piperidin-1- | | |
| | yl)ethanone | | |
| 27 | 3-(9-((1-benzylpiperidin-4-yl)methyl)-6- | 193 | 0.6 |
| | morpholino-9H-purin-2-yl)phenol | | |
| 28 | 3-(9-(1-benzylpiperidin-4-yl)-6- | 75 | 0.14 |
| | morpholino-9H-purin-2-yl)phenol | | |
| 29 | (3-(9-((1-benzylpiperidin-4-yl)methyl)-6- | 243 | 10.5 |
| | morpholino-9H-purin-2- | | |
| | yl)phenyl)methanol | | |
| 31 | 5-(9-(1-benzylpiperidin-4-yl)-6- | 43 | 0.22 |
| | morpholino-9H-purin-2-yl)pyrimidin-2- | | |
| | amine | | |
| 32 | 5-[9-(1-benzylpiperidin-4-yl)-6-morpholin- | 160 | 0.12 |
| | 4-yl-9H-purin-2-yl]pyridin-2-amine | | |
| 33 | {3-[9-(1-benzylpiperidin-4-yl)-6- | 45 | 0.700 |
| | morpholin-4-yl-9H-purin-2- | | |
| | yl]phenyl}methanol | | |
| 34 | 5-[9-(1-benzylpiperidin-4-yl)-6-morpholin- | 77 | 0.17 |
| | 4-yl-9H-purin-2-yl]nicotinaldehyde | | |
| 35 | {5-[9-(1-benzylpiperidin-4-yl)-6- | 63 | 1.0 |
| | morpholin-4-yl-9H-purin-2-yl]pyridin-3- | | |
| | yl}methanol | | |
| 36 | 5-(6-morpholin-4-yl-9-piperidin-4-yl-9H- | 14 | 0.97 |
| | purin-2-yl)pyridin-3-ol | | |
| 37 | 5-(9-{1-[(5-methyl-2- | 26 | 0.14 |
| | thienyl)methyl]piperidin-4-yl}-6- | | |
| | morpholin-4-yl-9H-purin-2-yl)pyridin-3-ol | | |
| 38 | 5-{9-[1-(4-chlorobenzyl)piperidin-4-yl]-6- | 16 | 0.16 |
| | morpholin-4-yl-9H-purin-2-yl}pyridin-3-ol | | |
| 39 | 5-{6-morpholin-4-yl-9-[1-(1H-pyrrol-2- | 31 | 0.94 |
| | ylmethyl)piperidin-4-yl]-9H-purin-2- | | |
| | yl}pyridin-3-ol | | |
| 40 | 5-{9-[1-(4-methylbenzyl)piperidin-4-yl]-6- | 42 | 0.59 |
| | morpholin-4-yl-9H-purin-2-yl}pyridin-3-ol | | |
| 41 | 5-{9-[1-(6-fluoro-pyridin-3-ylmethyl)- | 94 | 0.57 |
| | piperidin-4-yl]-6-morpholin-4-yl-9H-purin- | | |
| | 2-yl}-pyridin-3-ol | | |
| 42 | 2-(5-methoxypyridin-3-yl)-6-morpholin-4- | 598 | 3 |
| | yl-9H-purine | | |
| 43 | 5-(6-morpholin-4-yl-9H-purin-2-yl)pyridin- | 11 | 0.62 |
| | 3-ol | | |
| 44 | (3-{6-morpholin-4-yl-9-[1-(pyridin-2- | 65 | 0.31 |
| | ylmethyl)piperidin-4-yl]-9H-purin-2- | | |
| | yl}phenyl)methanol | | |
| 45 | (3-{9-[1-(2-fluorobenzyl)piperidin-4-yl]-6- | 42 | 0.16 |
| | morpholin-4-yl-9H-purin-2- | | |
| | yl}phenyl)methanol | | |
| 46 | (3-{9-[1-(4-fluorobenzyl)piperidin-4-yl]-6- | 70 | 1.7 |
| | morpholin-4-yl-9H-purin-2- | | |
| | yl}phenyl)methanol | | |
| 47 | (3-{6-morpholin-4-yl-9-[1-(4-pyridin-4- | 23 | 0.72 |
| | ylbenzyl)piperidin-4-yl]-9H-purin-2- | | |
| | yl}phenyl)methanol | | |
| 48 | {3-[9-(1-butylpiperidin-4-yl)-6-morpholin- | 96 | 4.2 |
| | 4-yl-9H-purin-2-yl]phenyl}methanol | | |
| 49 | (3-{9-[1-(2,4-difluorobenzyl)piperidin-4- | 44 | 2.05 |
| | yl]-6-morpholin-4-yl-9H-purin-2- | | |
| | yl}phenyl)methanol | | |
| 50 | (3-{9-[1-(3-fluorobenzyl)piperidin-4-yl]-6- | 94 | 1.4 |
| | morpholin-4-yl-9H-purin-2- | | |
| | yl}phenyl)methanol | | |
| 51 | {3-[9-(1-benzylpiperidin-4-yl)-6- | 63 | 1 |
| | morpholin-4-yl-9H-purin-2- | | |
| | yl]phenyl}methanol | | |
| 52 | (3-{9-[1-(2-furylmethyl)piperidin-4-yl]-6- | 46 | 0.49 |
| | morpholin-4-yl-9H-purin-2- | | |
| | yl}phenyl)methanol | | |
| 53 | 4-(2-(3-methoxyphenyl)-9H-purin-6- | | >20.000 |
| | yl)morpholine | | |
| 54 | 4-(2-phenyl-9H-purin-6-yl)morpholine] | | 19 |
| 55 | 3-(6-morpholino-9H-purin-2-yl)phenol | 264 | 2.125 |
| 56 | tert-butyl 4-(2-choro-6-morpholino-9H- | | |
| | purin-9-yl)piperidine-1-carboxylate | | |
| 57 | tert-butyl 4-{2-(3-hydroxyphenyl)-6- | 288 | 0.27 |
| | morpholino-9H-purin-9-yl}piperidine-1- | | |
| | carboxylate | | |
| 58 | tert-butyl-4-{2-[5- | 2093 | >4.000 |
| | (methoxymethoxy)pyridin-3-yl]-6- | | |
| | morpholin-4-yl-9H-purin-9-yl}piperidine- | | |
| | 1-carboxylate | | |
| 59 | tert-butyl 4-{2-[3-(hydroxymethyl)phenyl]- | 95 | 1.4 |
| | 6-morpholin-4-yl-9H-purin-9- | | |
| | yl}piperidine-1-carboxylate | | |
| 60 | (3-{6-morpholin-4-yl-9-[1-(2,4,6- | 194 | 3.3 |
| | trifluorobenzyl)piperidin-4-yl]-9H-purin-2- | | |
| | yl}phenyl)methanol | | |
| 61 | [3-(9-{1-[(6-fluoropyridin-3- | 47 | 0.71 |
| | yl)methyl]piperidin-4-yl}-6-morpholin-4- | | |
| | yl-9H-purin-2-yl)phenyl]methanol | | |
| 62 | (3-{9-[1-(3,4-difluorobenzyl)piperidin-4- | 42 | 1.2 |
| | yl]-6-morpholin-4-yl-9H-purin-2- | | |
| | yl}phenyl)methanol | | |
| 63 | [3-(9-{1-[(6-chloropyridin-3- | 45 | 0.65 |
| | yl)methyl]piperidin-4-yl}-6-morpholin-4- | | |
| | yl-9H-purin-2-yl)phenyl]methanol | | |
| 64 | [3-(9-{1-[(6-methoxypyridin-3- | 168 | >4.000 |
| | yl)methyl]piperidin-4-yl}-6-morpholin-4- | | |
| | yl-9H-purin-2-yl)phenyl]methanol | | |
| 65 | [3-(9-{1-[(2,6-dimethoxypyridin-3- | 127 | >4.000 |
| | yl)methyl]piperidin-4-yl}-6-morpholin-4- | | |
| | yl-9H-purin-2-yl)phenyl]methanol | | |
| 66 | [3-(9-{1-[(5-fluoropyridin-3- | 177 | >4.000 |
| | yl)methyl]piperidin-4-yl}-6-morpholin-4- | | |
| | yl-9H-purin-2-yl)phenyl]methanol | | |
| 67 | [3-(9-{1-[(5-methyl-2- | 518 | >4.000 |
| | thienyl)methyl]piperidin-4-yl}-6- | | |
| | morpholin-4-yl-9H-purin-2- | | |
| | yl)phenyl]methanol | | |
| 68 | (3-{6-morpholin-4-yl-9-[1-(1H-pyrrol-2- | 37 | 2.5 |
| | ylmethyl)piperidin-4-yl]-9H-purin-2- | | |
| | yl}phenyl)methanol | | |
| 69 | (3-{9-[1-(2-chloro-4- | 77 | 1.55 |
| | fluorobenzyl)piperidin-4-yl]-6-morpholin- | | |
| | 4-yl-9H-purin-2-yl}phenyl)methanol | | |
| 70 | (3-{9-[1-(1H-imidazol-2- | 152 | 1.5 |
| | ylmethyl)piperidin-4-yl]-6-morpholin-4-yl- | | |
| | 9H-purin-2-yl}phenyl)methanol | | |
| 71 | [3-(9-{1-[(6-bromopyridin-2- | 50 | 0.58 |
| | yl)methyl]piperidin-4-yl}-6-morpholin-4- | | |
| | yl-9H-purin-2-yl)phenyl]methanol | | |
| 72 | [3-(6-morpholin-4-yl-9-{1-[(6-morpholin- | 99 | 1.8 |
| | 4-ylpyridin-2-yl)methyl]piperidin-4-yl}- | | |
| | 9H-purin-2-yl)phenyl]methanol | | |
| 73 | [3-(9-{1-[(5-fluoro-1H-indol-3- | 30 | 1.3 |
| | yl)methyl]piperidin-4-yl}-6-morpholin-4- | | |
| | yl-9H-purin-2-yl)phenyl]methanol | | |
| 74 | (3-{9-[1-(5,6-dihydro-8H-imidazo[2,1- | 94 | 2 |
| | c][1,4]oxazin-3-ylmethyl)piperidin-4-yl]-6- | | |
| | morpholin-4-yl-9H-purin-2- | | |
| | yl}phenyl)methanol | | |
| 75 | {3-[9-(1-{4-[3- | 16 | >4.000 |
| | dimethylamino)propoxy]benzyl}piperidin- | | |
| | 4-yl)-6-morpholin-4-yl-9H-purin-2- | | |
| | yl]phenyl}methanol | | |
| 76 | 3-{6-morpholin-4-yl-9-[1-(pyridin-3- | 66 | 0.17 |
| | ylmethyl)piperidin-4-yl]-9H-purin-2- | | |
| | yl}phenol | | |
| 77 | 3-{6-morpholin-4-yl-9-[1-(pyridin-2- | 156 | 0.29 |
| | ylmethyl)piperidin-4-yl]-9H-purin-2- | | |
| | yl}phenol | | |
| 78 | 3-(9-{1-[(6-chloropyridin-3- | 37 | 0.2 |
| | yl)methyl]piperidin-4-yl}-6-morpholin-4- | | |
| | yl-9H-purin-2-yl)phenol | | |
| 79 | 3-(9-{1-[(6-methoxypyridin-3- | 55 | 0.051 |
| | yl)methyl]piperidin-4-yl}-6-morpholin-4- | | |
| | yl-9H-purin-2-yl)phenol | | |
| 80 | 3-(9-{1-[(2,6-dimethoxypyridin-4- | 75 | 0.17 |
| | yl)methyl]piperidin-4-yl}-6-morpholin-4- | | |
| | yl-9H-purin-2-yl)phenol | | |
| 81 | 3-(9-{1-[(6-bromopyridin-3- | 49 | 0.12 |
| | yl)methyl]piperidin-4-yl}-6-morpholin-4- | | |
| | yl-9H-purin-2-yl)phenol | | |
| 82 | 3-(6-morpholin-4-yl-9-{1-[(6-morpholin-4- | 55 | 0.145 |
| | ylpyridin-3-yl)methyl]piperidin-4-yl}-9H- | | |
| | purin-2-yl)phenol | | |
| 83 | 3-[9-(1-{[4-(dimethylamino)-1- | 83 | 0.36 |
| | naphthyl]methyl}piperidin-4-yl)-6- | | |
| | morpholin-4-yl-9H-purin-2-yl]phenol | | |
| 84 | 3-(9-{1-[(6-fluoropyridin-3- | 70 | 0.31 |
| | yl)methyl]piperidin-4-yl}-6-morpholin-4- | | |
| | yl-9H-purin-2-yl)phenol | | |
| 85 | 3-[6-morpholin-4-yl-9-(2-piperidin-1- | 163 | 9.8 |
| | ylethyl)-9H-purin-2-yl]phenol | | |
| 86 | {3-[6-morpholin-4-yl-9-(2-piperidin-1- | 91 | 13 |
| | ylethyl)-9H-purin-2-yl]phenyl}methanol | | |
| 87 | 5-[9-(1-benzylpiperidin-4-yl)-6-morpholin- | 87 | 0.55 |
| | 4-yl-9H-purin-2-yl]pyridin-3-ol | | |
| 88 | 5-(9-{1-[(6-fluoropyridin-3- | 85 | 0.50 |
| | yl)methyl]piperidin-4-yl}-6-morpholin-4- | | |
| | yl-9H-purin-2-yl)pyridin-3-ol | | |
| 89 | 1-methyl-3-(4-(6-morpholino-9-(1- | 340 | 0.0097 |
| | (pyridin-3-ylmethyl)piperidin-4-yl)-9H- | | |
| | purin-2-yl)phenyl)urea | | |
| 90 | 1-ethyl-3-(4-(6-morpholino-9-(1-(pyridin- | 1350 | 0.012 |
| | 3-ylmethyl)piperidin-4-yl)-9H-purin-2- | | |
| | yl)phenyl)urea | | |
| 91 | 1-(2-(3-hydroxyphenyl)-6-morpholino-9H- | 43 | 0.073 |
| | purin-9-yl)prop-2-en-1-one | | |

Throughout this application, various publications are referenced. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure, as it appears in the U.S. Patent and Trademark Office patent file or records, but otherwise reserves any and all copyright rights whatsoever.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A compound of Formula Ib: 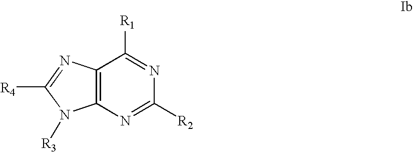
and pharmaceutically acceptable salts thereof, wherein:
R₁ is N-morpholinyl or N-thiomorpholinyl, wherein the N-thiomorpholinyl sulfur atom may be substituted with one or two ═O, wherein any one or more of the ring hydrogen atoms of the N-morpholinyl or N-thiomorpholinyl can independently be substituted with C₁-C₆alkyl, C₂-C₆alkenyl, C₂-C₆alkynyl, C₁-C₃alkoxy, C₁-C₃acyl, C₁-C₃alkylcarboxy, (C₁-C₆alkyl)amino, fluorine, or —CN;
where any two hydrogen atoms attached to the same carbon atom in R₁ can be replaced by an oxygen atom, where the oxygen atom taken together with the carbon to which it is attached, forms a carbonyl (C═O);
R₂ is
(a) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
(b) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
(c) C₆-C₁₄aryl optionally substituted with from 1 to 3 substituents independently selected from:
(i) halogen,
(ii) C₁-C₆alkyl,
(iii) C₁-C₆alkoxy, optionally substituted with C₁-C₆alkoxy,
(iv) C₁-C₆hydroxylalkyl,
(v) C₂-C₆alkenyl,
(vi) C₂-C₆alkynyl,
(vii) C₃-C₈carbocycle,
(viii) C₆-C₁₄aryl,
(ix) C₁-C₉heteroaryl,
(x) C₁-C₆perfluoroalkyl-,
(xi) hydroxyl,
(xii) NR₁₂R₁₂,
(xiii) NO₂,
(xiv) CN,
(xv) CO₂H,
(xvi) CHO,
(xvii) C₆-C₁₄aryl-O—,
(xviii) (C₆-C₁₄aryl)alkyl-O—, optionally substituted with from 1 to 3 C₁-C₆alkoxy,
(xix) —C(O)NR₁₂R₁₂,
(xx) NHC(O)R₁₃,
(xxi) —NHC(O)NR₁₂R₁₂,
(xxii) —NHC(O)OR₁₃,
(xxiii) —NH(SO₂)—(C₁-C₆alkyl),
(xxiv) and —(SO₂)—(C₁-C₆alkyl);
where any two hydrogen atoms on adjacent carbon atoms of the C₆-C₁₄aryl can be replaced by an alkylenedioxy group so that the alkylenedioxy group, when taken together with the two carbon atoms to which it is attached, form a 5- to 7-membered heterocycle containing two oxygen atoms;
(d) or C₁-C₉heteroaryl optionally substituted with from 1 to 3 substituents independently selected from:
(i) halogen,
(ii) C₁-C₆alkyl,
(iii) C₁-C₆alkoxy, optionally substituted with C₁-C₆alkoxy,
(iv) C₁-C₆hydroxylalkyl,
(v) C₂-C₆alkenyl,
(vi) C₂-C₆alkynyl,
(vii) C₃-C₈carbocycle,
(viii) C₆-C₁₄aryl,
(ix) C₁-C₉heteroaryl,
(x) C₁-C₆perfluoroalkyl-,
(xi) hydroxyl,
(xii) NR₁₂R₁₂,
(xiii) NO₂,
(xiv) CN,
(xv) CO₂H,
(xvi) CHO,
(xvii) C₆-C₁₄aryl-O—,
(xviii) (C₆-C₁₄aryl)alkyl-O—, optionally substituted with from 1 to 3 C₁-C₆alkoxy,
(xix) —C(O)NR₁₂R₁₂,
(xx) NHC(O)R₁₃,
(xxi) —NHC(O)NR₁₂R₁₂,
(xxii) —NHC(O)OR₁₃,
(xxiii) —NH(SO₂)—(C₁-C₆alkyl),
(xxiv) and —(SO₂)—(C₁-C₆alkyl);
where any two hydrogen atoms on adjacent carbon atoms of the C₁-C₉heteroaryl can be replaced by an alkylenedioxy group so that the alkylenedioxy group, when taken together with the two carbon atoms to which it is attached, form a 5- to 7-membered heterocycle containing two oxygen atoms;
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl;
R₃ is:
(a) hydrogen;
(b) C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(c) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
(d) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
(e) C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(f) C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(g) C₃-C₈carbocycle, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(h) heterocyclyl(C₁-C₆alkyl) optionally substituted with (C₆-C₁₄aryl)alkyl;
(i) 3- to 7-membered monocyclic heterocycle optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(v) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl;
(i) —C(O)OH,
(ii) halogen,
(iii) —NH₂,
(iv) —NH(C₁-C₆alkyl),
(v) —N(C₁-C₆alkyl)(C₁-C₆alkyl),
(vi) —N(C₁-C₃alkyl)C(O)(C₁-C₆alkyl),
(vii) —NHC(O)(C₁-C₆alkyl),
(viii) —NHC(O)H,
(ix) —C(O)NH₂,
(x) —C(O)NH(C₁-C₆alkyl),
(xi) —C(O)N(C₁-C₆alkyl)(C₁-C₆alkyl),
(xii) —CN,
(xiii) —OH,
(xiv) —O(C₁-C₆alkyl),
(xv) C₆-C₁₄aryl,
(xvi) C₁-C₉heteroaryl,
(xvii) and C₃-C₈carbocycle;
(j) nitrogen containing 3- to 7-membered monocyclic heterocycle optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(ii) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(iii) —C(O)OH,
(iv) halogen,
(v) —NH₂,
(vi) —NH(C₁-C₆alkyl),
(vii) —N(C₁-C₆alkyl)(C₁-C₆alkyl),
(viii) —N(C₁-C₃alkyl)C(O)(C₁-C₆alkyl),
(ix) —NHC(O)(C₁-C₆alkyl),
(x) —NHC(O)H,
(xi) —C(O)NH₂,
(xii) —C(O)NH(C₁-C₆alkyl),
(xiii) —C(O)N(C₁-C₆alkyl)(C₁-C₆alkyl),
(xiv) —CN,
(xv) —OH,
(xvi) —O(C₁-C₆alkyl),
(xvii) C₆-C₁₄aryl,
(xviii) C₁-C₉heteroaryl,
(xix) and C₃-C₈carbocycle;
(k) 7- to 10-membered bicyclic heterocycle optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(ii) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(iii) —C(O)OH,
(iv) halogen,
(v) —NH₂,
(vi) —NH(C₁-C₆alkyl),
(vii) —N(C₁-C₆alkyl)(C₁-C₆alkyl),
(viii) —N(C₁-C₃alkyl)C(O)(C₁-C₆alkyl),
(ix) —NHC(O)(C₁-C₆alkyl),
(x) —NHC(O)H,
(xi) —C(O)NH₂,
(xii) —C(O)NH(C₁-C₆alkyl),
(xiii) —C(O)N(C₁-C₆alkyl)(C₁-C₆alkyl),
(xiv) —CN,
(xv) —OH,
(xvi) —O(C₁-C₆alkyl),
(xvii) C₆-C₁₄aryl,
(xviii) C₁-C₉heteroaryl,
(xix) and C₃-C₈carbocycle;
(l) or nitrogen-containing 7- to 10-membered bicyclic heterocycle, wherein the nitrogen of the nitrogen containing 3- to 7-membered monocyclic heterocycle is optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl;
(ii) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(iii) —C(O)OH,
(iv) halogen,
(v) —NH₂,
(vi) —NH(C₁-C₆alkyl),
(vii) —N(C₁-C₆alkyl)(C₁-C₆alkyl),
(viii) —N(C₁-C₃alkyl)C(O)(C₁-C₆alkyl),
(ix) —NHC(O)(C₁-C₆alkyl),
(x) —NHC(O)H,
(xi) —C(O)NH₂,
(xii) —C(O)NH(C₁-C₆alkyl),
(xiii) —C(O)N(C₁-C₆alkyl)(C₁-C₆alkyl),
(xiv) —CN,
(xv) —OH,
(xvi) —O(C₁-C₆alkyl),
(xvii) C₆-C₁₄aryl,
(xviii) C₁-C₉heteroaryl,
(xix) and C₃-C₈carbocycle;
(m) C₁-C₆hydroxylalkyl;
(n) C₁-C₆alkylcarboxy;
(o) C₁-C₆perfluoroalkyl;
(p) —S(O)_{q}—C₁-C₆alkyl;
(q) or —S(O)_{q}—C₆-C₄aryl;
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
q is 0, 1 or 2;
with the proviso that R₃ and R₄ cannot simultaneously be unsubstituted C₁-C₆alkyl;
and that 3-[6-(4-morpholinyl)-9H-purin-2-yl]-phenol is excluded.

2. The compound of claim 1 wherein R₁ is N-morpholinyl.

3. The compound of claim 1 wherein, R₂ is C₆-C₁₄aryl optionally substituted with from 1 to 3 substituents as specified in claim 1.

4. The compound of claim 1 wherein R₂ is C₁-C₉heteroaryl optionally substituted with from 1 to 3 substituents as specified in claim 1.

5. The compound of claim 1 wherein R₂ is C₆-C₁₄aryl substituted with from 1 to 3 —NHC(O)NR₁₂R₁₂.

6. The compound of claim 1 wherein R₂ is C₆-C₁₄aryl substituted from 1 to 3 NHC(O)R₁₃.

7. The compound of claim 1 wherein R₃ is hydrogen.

8. The compound of claim 1 wherein R₃ is C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

9. The compound of claim 1 wherein R₃ is C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

10. The compound of claim 1 wherein R₃ is C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

11. The compound of claim 1 wherein R₃ is —S(O)_{q}—C₁-C₆ alkyl.

12. The compound of claim 1 wherein R₃ is —S(O)_{q}-aryl.

13. The compound of claim 1 wherein R₃ is a 3- to 7-membered monocyclic heterocycle, optionally substituted with from 1 to 3 substituents as specified in claim 1.

14. The compound of claim 1 wherein R₃ is 7- to 10-membered bicyclic heterocycle optionally substituted with from 1 to 3 substituents as specified in claim 1.

15. The compound of claim 11 wherein q is 0.

16. The compound of claim 11 wherein q is 1.

17. The compound of claim 11 wherein q is 2.

18. A compound of Formula Ic: 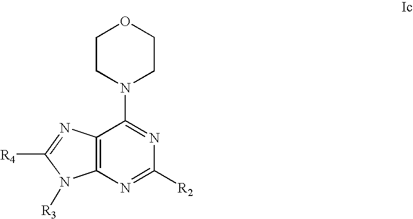
and pharmaceutically acceptable salts thereof, wherein
R₂ is
(a) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
(b) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
(c) C₆-C₁₄aryl optionally substituted with from 1 to 3 substituents independently selected from:
(i) halogen,
(ii) C₁-C₆alkyl,
(iii) C₁-C₆alkoxy, optionally substituted with C₁-C₆alkoxy,
(iv) C₁-C₆hydroxylalkyl,
(v) C₂-C₆alkenyl,
(vi) C₂-C₆alkynyl,
(vii) C₃-C₈carbocycle,
(viii) C₆-C₁₄aryl,
(ix) C₁-C₉heteroaryl,
(x) C₁-C₆perfluoroalkyl-,
(xi) hydroxyl,
(xii) NR₁₂R₁₂,
(xiii) NO₂,
(xiv) CN,
(xv) CO₂H,
(xvi) CHO,
(xvii) C₆-C₁₄aryl-O—,
(xviii) (C₆-C₁₄aryl)alkyl-O—, optionally substituted with from 1 to 3 C₁-C₆alkoxy,
(xix) —C(O)NR₁₂R₁₂,
(xx) NHC(O)R₁₃,
(xxi) —NHC(O)NR₁₂R₁₂,
(xxii) —NHC(O)OR₁₃,
(xxiii) —NH(SO₂)—(C₁-C₆alkyl),
(xxiv) and —(SO₂)—(C₁-C₆alkyl);
where any two hydrogen atoms on adjacent carbon atoms of the C₆-C₁₄aryl can be replaced by an alkylenedioxy group so that the alkylenedioxy group, when taken together with the two carbon atoms to which it is attached, form a 5- to 7-membered heterocycle containing two oxygen atoms;
(d) or C₁-C₉heteroaryl optionally substituted with from 1 to 3 substituents independently selected from:
(i) halogen,
(ii) C₁-C₆alkyl,
(iii) C₁-C₆alkoxy, optionally substituted with C₁-C₆alkoxy,
(iv) C₁-C₆hydroxylalkyl,
(v) C₂-C₆alkenyl,
(vi) C₂-C₆alkynyl,
(vii) C₃-C₈carbocycle,
(viii) C₆-C₁₄aryl,
(ix) C₁-C₉heteroaryl,
(x) C₁-C₆perfluoroalkyl-,
(xi) hydroxyl,
(xii) NR₁₂R₁₂,
(xiii) NO₂,
(xiv) CN,
(xv) CO₂H,
(xvi) CHO,
(xvii) C₆-C₁₄aryl-O—,
(xviii) (C₆-C₁₄aryl)alkyl-O—, optionally substituted with from 1 to 3 C₁-C₆alkoxy,
(xix) —C(O)NR₁₂R₁₂,
(xx) NHC(O)R₁₃,
(xxi) —NHC(O)NR₁₂R₁₂,
(xxii) —NHC(O)OR₁₃,
(xxiii) —NH(SO₂)—(C₁-C₆alkyl),
(xxiv) and —(SO₂)—(C₁-C₆alkyl);
where any two hydrogen atoms on adjacent carbon atoms of the C₁-C₉heteroaryl can be replaced by an alkylenedioxy group so that the alkylenedioxy group, when taken together with the two carbon atoms to which it is attached, form a 5- to 7-membered heterocycle containing two oxygen atoms;
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl;
R₃ is:
(a) hydrogen;
(b) C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(c) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
(d) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
(e) C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(f) C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(g) C₃-C₈carbocycle, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle;
(h) heterocyclyl(C₁-C₆alkyl) optionally substituted with (C₆-C₁₄aryl)alkyl;
(i) 3- to 7-membered monocyclic heterocycle optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(v) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl;
(vi) —C(O)OH,
(vii) halogen,
(viii) —NH₂,
(ix) —NH(C₁-C₆alkyl),
(x) —N(C₁-C₆alkyl)(C₁-C₆alkyl),
(xi) —N(C₁-C₃alkyl)C(O)(C₁-C₆alkyl),
(xii) —NHC(O)(C₁-C₆alkyl),
(xiii) —NHC(O)H,
(xiv) —C(O)NH₂,
(xv) —C(O)NH(C₁-C₆alkyl),
(xvi) —C(O)N(C₁-C₆alkyl)(C₁-C₆alkyl),
(xvii) —CN,
(xviii) —OH,
(xix) —O(C₁-C₆alkyl),
(xx) C₆-C₁₄aryl,
(xxi) C₁-C₉heteroaryl,
(xxii) and C₃-C₈carbocycle;
(j) nitrogen containing 3- to 7-membered monocyclic heterocycle optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(v) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(vi) —C(O)OH,
(vii) halogen,
(viii) —NH₂,
(ix) —NH(C₁-C₆alkyl),
(x) —N(C₁-C₆alkyl)(C₁-C₆alkyl),
(xi) —N(C₁-C₃alkyl)C(O)(C₁-C₆alkyl),
(xii) —NHC(O)(C₁-C₆alkyl),
(xiii) —NHC(O)H,
(xiv) —C(O)NH₂,
(xv) —C(O)NH(C₁-C₆alkyl),
(xvi) —C(O)N(C₁-C₆alkyl)(C₁-C₆alkyl),
(xvii) —CN,
(xviii) —OH,
(xix) —O(C₁-C₆alkyl),
(xx) C₆-C₁₄aryl,
(xxi) C₁-C₉heteroaryl,
(xxii) and C₃-C₈carbocycle;
(xxiii)
(k) 7- to 10-membered bicyclic heterocycle optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(v) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(vi) —C(O)OH,
(vii) halogen,
(viii) —NH₂,
(ix) —NH(C₁-C₆alkyl),
(x) —N(C₁-C₆alkyl)(C₁-C₆alkyl),
(xi) —N(C₁-C₃alkyl)C(O)(C₁-C₆alkyl),
(xii) —NHC(O)(C₁-C₆alkyl),
(xiii) —NHC(O)H,
(xiv) —C(O)NH₂,
(xv) —C(O)NH(C₁-C₆alkyl),
(xvi) —C(O)N(C₁-C₆alkyl)(C₁-C₆alkyl),
(xvii) —CN,
(xviii) —OH,
(xix) —O(C₁-C₆alkyl),
(xx) C₆-C₁₄aryl,
(xxi) C₁-C₉heteroaryl,
(xxii) and C₃-C₈carbocycle;
(l) or nitrogen-containing 7- to 10-membered bicyclic heterocycle, wherein the nitrogen of the nitrogen containing 3- to 7-membered monocyclic heterocycle is optionally substituted with one or more substituent independently selected from:
(i) C₁-C₆alkyl,
(ii) (C₁-C₆alkoxy)carbonyl,
(iii) C₁-C₈acyl,
(iv) (C₆-C₁₄aryl)alkyl, wherein the ring portion of the (C₆-C₁₄aryl)alkyl group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl;
(v) heteroaryl(C₁-C₆alkyl), wherein the ring portion of the heteroaryl(C₁-C₆alkyl) group is optionally substituted by 1 to 3 substituents independently selected from:
A) halogen,
B) C₁-C₆alkyl,
C) NH₂,
D) (C₁-C₆alkyl)amino,
E) di(C₁-C₆alkyl)amino,
F) C₁-C₆alkoxy, wherein the C₁-C₆alkoxy is optionally substituted with NH₂, (C₁-C₆alkyl)amino, or di(C₁-C₆alkyl)amino,
G) C₁-C₉heterocycle,
H) C₆-C₁₄aryl,
I) and C₁-C₉heteroaryl,
(vi) —C(O)OH,
(vii) halogen,
(viii) —NH₂,
(ix) —NH(C₁-C₆alkyl),
(x) —N(C₁-C₆alkyl)(C₁-C₆alkyl),
(xi) —N(C₁-C₃alkyl)C(O)(C₁-C₆alkyl),
(xii) —NHC(O)(C₁-C₆alkyl),
(xiii) —NHC(O)H,
(xiv) —C(O)NH₂,
(xv) —C(O)NH(C₁-C₆alkyl),
(xvi) —C(O)N(C₁-C₆alkyl)(C₁-C₆alkyl),
(xvii) —CN,
(xviii) —OH,
(xix) —O(C₁-C₆alkyl),
(xx) C₆-C₁₄aryl,
(xxi) C₁-C₉heteroaryl,
(xxii) and C₃-C₈carbocycle;
(m) C₁-C₆hydroxylalkyl;
(n) C₁-C₆alkylcarboxy;
(o) C₁-C₆perfluoroalkyl;
(p) —S(O)_{q}—C₁-C₆alkyl;
(q) or —S(O)_{q}—C₆-C₄aryl;
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
q is 0, 1 or 2;
with the proviso that R₃ and R₄ cannot simultaneously be unsubstituted C₁-C₆alkyl;
and that 3-[6-(4-morpholinyl)-9H-purin-2-yl]-phenol is excluded.

19. The compound of claim 18, wherein R₁ is N-morpholinyl.

20. The compound of claim 18, wherein R₂ is C₆-C₁₄aryl optionally substituted with from 1 to 3 substituents as specified in claim 18.

21. The compound of claim 18, wherein R₂ is C₁-C₉heteroaryl optionally substituted with from 1 to 3 substituents as specified in claim 18.

22. The compound of claim 18, wherein R₂ is C₆-C₁₄aryl substituted with from 1 to 3 —NHC(O)NR₁₂R₁₂.

23. The compound of claim 18, wherein R₂ is C₆-C₁₄aryl substituted from 1 to 3 NHC(O)R₁₃.

24. The compound of claim 18, wherein R₃ is hydrogen.

25. The compound of claim 18, wherein R₃ is C₁-C₆alkyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

26. The compound of claim 18, wherein R₃ is C₆-C₁₄aryl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

27. The compound of claim 18, wherein R₃ is C₁-C₉heteroaryl optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle.

28. The compound of claim 18, wherein R₃ is —S(O)_{q}—C₁-C₆ alkyl.

29. The compound of claim 18, wherein R₃ is —S(O)_{q}-aryl.

30. The compound of claim 18, wherein R₃ is a 3- to 7-membered monocyclic heterocycle, optionally substituted with from 1 to 3 substituents as specified in claim 18.

31. The compound of claim 18, wherein R₃ is 7- to 10-membered bicyclic heterocycle optionally substituted with from 1 to 3 substituents as specified in claim 18.

32. The compound of claim 28, wherein q is 0.

33. The compound of claim 28, wherein q is 1.

34. The compound of claim 28, wherein q is 2.

35. A compound of Formula II: 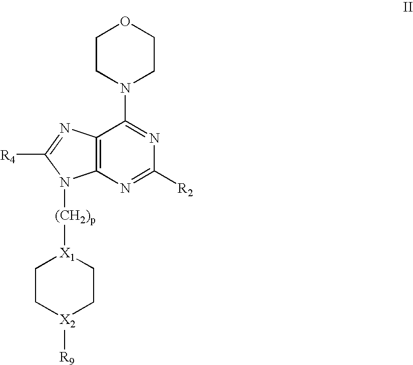
and pharmaceutically acceptable salts thereof; wherein
X₁ is —C(H)— or —N—;
X₂ is —C(H), —N—, —O—, or —S(O)ₙ—, provided that when X₂ is —O— or —S(O)ₙ—, R₉ is absent;
n is 0, 1, or 2;
p is 0, 1, 2, 3, 4, or 5, provided that when p is 0 a bond exists between the —N— and X₁ and X₁ cannot be —N—;
R₂ is:
(a) C₂-C₁₀alkenyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the double bond;
(b) C₂-C₁₀alkynyl, optionally substituted with one or more substituent independently selected from halogen, —NH₂, —NH(C₁-C₆alkyl), —N(C₁-C₆alkyl)(C₁-C₆alkyl), —N(C₁-C₃alkyl)C(O)(C₁-C₆alkyl), —NHC(O)(C₁-C₆alkyl), —NHC(O)H, —C(O)NH₂, —C(O)NH(C₁-C₆alkyl), —C(O)N(C₁-C₆alkyl)(C₁-C₆alkyl), —CN, —OH, —O(C₁-C₆alkyl), —C₁-C₆alkyl, —C(O)OH, —C(O)OC₁-C₆alkyl, —C(O)C₁-C₆alkyl, C₆-C₁₄aryl, C₁-C₉heteroaryl, and C₃-C₈carbocycle, provided that the substituent is not attached to a carbon of the triple bond;
(c) C₆-C₁₄aryl optionally substituted with from 1 to 3 substituents independently selected from:
(i) halogen,
(ii) C₁-C₆alkyl,
(iii) C₁-C₆alkoxy, optionally substituted with C₁-C₆alkoxy,
(iv) C₁-C₆hydroxylalkyl,
(v) C₂-C₆alkenyl,
(vi) C₂-C₆alkynyl,
(vii) C₃-C₈carbocycle,
(viii) C₆-C₁₄aryl,
(ix) C₁-C₉heteroaryl,
(x) C₁-C₆perfluoroalkyl-,
(xi) hydroxyl,
(xii) NR₁₂R₁₂,
(xiii) NO₂,
(xiv) CN,
(xv) CO₂H,
(xvi) CHO,
(xvii) C₆-C₁₄aryl-O—,
(xviii) (C₆-C₁₄aryl)alkyl-O—, optionally substituted with from 1 to 3 C₁-C₆alkoxy,
(xix) —C(O)NR₁₂R₁₂,
(xx) NHC(O)R₁₃,
(xxi) —NHC(O)NR₁₂R₁₂,
(xxii) —NHC(O)OR₁₃,
(xxiii) —NH(SO₂)—(C₁-C₆alkyl),
(xxiv) and —(SO₂)—(C₁-C₆alkyl);
where any two hydrogen atoms on adjacent carbon atoms of the C₆-C₁₄aryl can be replaced by an alkylenedioxy group so that the alkylenedioxy group, when taken together with the two carbon atoms to which it is attached, form a 5- to 7-membered heterocycle containing two oxygen atoms;
(d) or C₁-C₉heteroaryl optionally substituted with from 1 to 3 substituents independently selected from:
(i) halogen,
(ii) C₁-C₆alkyl,
(iii) C₁-C₆alkoxy, optionally substituted with C₁-C₆alkoxy,
(iv) C₁-C₆hydroxylalkyl,
(v) C₂-C₆alkenyl,
(vi) C₂-C₆alkynyl,
(vii) C₃-C₈carbocycle,
(viii) C₆-C₁₄aryl,
(ix) C₁-C₉heteroaryl,
(x) C₁-C₆perfluoroalkyl-,
(xi) hydroxyl,
(xii) NR₁₂R₁₂,
(xiii) NO₂,
(xiv) CN,
(xv) CO₂H,
(xvi) CHO,
(xvii) C₆-C₁₄aryl-O—,
(xviii) (C₆-C₁₄aryl)alkyl-O—, optionally substituted with from 1 to 3 C₁-C₆alkoxy,
(xix) —C(O)NR₁₂R₁₂,
(xx) NHC(O)R₁₃,
(xxi) —NHC(O)NR₁₂R₁₂,
(xxii) —NHC(O)OR₁₃,
(xxiii) —NH(SO₂)—(C₁-C₆alkyl),
(xxiv) and —(SO₂)—(C₁-C₆alkyl);
where any two hydrogen atoms on adjacent carbon atoms of the C₁-C₉heteroaryl can be replaced by an alkylenedioxy group so that the alkylenedioxy group, when taken together with the two carbon atoms to which it is attached, form a 5- to 7-membered heterocycle containing two oxygen atoms;
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl;
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
R₉ is:
(a) hydrogen;
(b) C₁-C₆alkyl;
(c) (C₁-C₆alkoxy)carbonyl;
(d) C₁-C₈acyl;
(e) (C₆-C₁₄aryl)alkyl, wherein the ring portion of the (C₆-C₁₄aryl)alkyl group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(f) heteroaryl(C₁-C₆alkyl), wherein the ring portion of the heteroaryl(C₁-C₆alkyl) group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(g) —C(O)OH;
(h) halogen;
(i) —NH₂;
(j) —NH(C₁-C₆alkyl);
(k) —N(C₁-C₆alkyl)(C₁-C₆alkyl);
(l) —N(C₁-C₃alkyl)C(O)(C₁-C₆alkyl);
(m) —NHC(O)(C₁-C₆alkyl);
(n) —NHC(O)H;
(o) —C(O)NH₂;
(p) —C(O)NH(C₁-C₆alkyl);
(q) —C(O)N(C₁-C₆alkyl)(C₁-C₆alkyl);
(r) —CN;
(s) —OH;
(t) —O(C₁-C₆alkyl);
(u) C₆-C₁₄aryl,
(v) C₁-C₉heteroaryl,
(w) or C₃-C₈carbocycle.

36. The compound of claim 35, wherein R₂ is C₆-C₁₄aryl optionally substituted with from 1 to 3 substituents as specified in claim 35.

37. The compound of claim 35, wherein R₂ is C₁-C₉heteroaryl optionally substituted with from 1 to 3 substituents as specified in claim 35.

38. The compound of claim 35, wherein R₂ is C₆-C₁₄aryl substituted with from 1 to 3 —NHC(O)NR₁₂R₁₂.

39. The compound of claim 35, wherein R₂ is C₆-C₁₄aryl substituted from 1 to 3 NHC(O)R₁₃.

40. The compound of claim 35, wherein R₄ is hydrogen.

41. The compound of claim 35, wherein X₁ is —N—.

42. The compound of claim 35, wherein X₁ is —C(H)—.

43. The compound of claim 35, wherein X₁ is —C(H)— and X₂ is —N—.

44. The compound of claim 35, wherein X₂ is —O— and R₉ is absent.

45. The compound of claim 35, wherein p is 0.

46. The compound of claim 35, wherein p is 1.

47. The compound of claim 35, wherein R₉ is:
(a) C₁-C₆alkyl;
(b) (C₁-C₆alkoxy)carbonyl;
(c) C₁-C₈acyl;
(d) (C₆-C₁₄aryl)alkyl, wherein the ring portion of the (C₆-C₁₄aryl)alkyl group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(e) heteroaryl(C₁-C₆alkyl), wherein the ring portion of the heteroaryl(C₁-C₆alkyl) group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(f) —C(O)OH;
(g) halogen;
(h) —NH₂;
(i) —NH(C₁-C₆alkyl);
(j) —N(C₁-C₆alkyl)(C₁-C₆alkyl);
(k) —N(C₁-C₃alkyl)C(O)(C₁-C₆alkyl);
(l) —NHC(O)(C₁-C₆alkyl);
(m) —NHC(O)H;
(n) —C(O)NH₂;
(o) —C(O)NH(C₁-C₆alkyl);
(p) —C(O)N(C₁-C₆alkyl)(C₁-C₆alkyl);
(q) —CN;
(r) —OH;
(s) —O(C₁-C₆alkyl);
(t) C₆-C₁₄aryl,
(u) C₁-C₉heteroaryl,
(v) or C₃-C₈carbocycle.

48. A compound of Formula Ia: 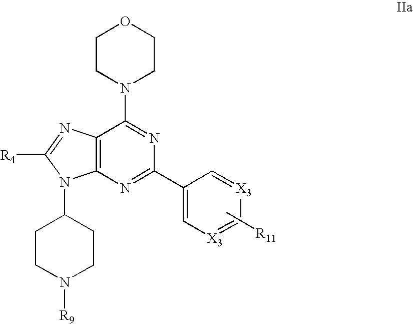
and pharmaceutically acceptable salts thereof, wherein
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
R₉ is:
(a) hydrogen;
(b) C₁-C₆alkyl;
(c) (C₁-C₆alkoxy)carbonyl;
(d) C₁-C₈acyl;
(e) (C₆-C₁₄aryl)alkyl, wherein the ring portion of the (C₆-C₁₄aryl)alkyl group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(f) heteroaryl(C₁-C₆alkyl), wherein the ring portion of the heteroaryl(C₁-C₆alkyl) group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(g) —C(O)OH;
(h) halogen;
(i) —NH₂;
(j) —NH(C₁-C₆alkyl);
(k) —N(C₁-C₆alkyl)(C₁-C₆alkyl);
(l) —N(C₁-C₃alkyl)C(O)(C₁-C₆alkyl);
(m) —NHC(O)(C₁-C₆alkyl);
(n) —NHC(O)H;
(o) —C(O)NH₂;
(p) —C(O)NH(C₁-C₆alkyl);
(q) —C(O)N(C₁-C₆alkyl)(C₁-C₆alkyl);
(r) —CN;
(s) —OH;
(t) —O(C₁-C₆alkyl);
(u) C₆-C₁₄aryl,
(v) C₁-C₉heteroaryl,
(w) or C₃-C₈carbocycle;
each X₃ is independently —N—, —C(H)—, —C((CH₂)_{w}—OH)—, or —C(C(O)H)—;
w is 0, 1, 2, 3, 4, or 5;
R₁₁ is
(a) halogen;
(b) C₁-C₆alkyl;
(c) C₁-C₆alkoxy; optionally substituted with C₁-C₆alkoxy;
(d) C₁-C₆hydroxylalkyl;
(e) C₂-C₆alkenyl;
(f) C₂-C₆alkynyl;
(g) C₃-C₈carbocycle;
(h) C₆-C₁₄aryl;
(i) C₁-C₉heteroaryl;
(j) C₁-C₆perfluoroalkyl-;
(k) hydroxyl;
(l) NR₁₂R₁₂;
(m) NO₂;
(n) CN;
(o) CO₂H;
(p) CHO;
(q) C₆-C₁₄aryl-O—;
(r) (C₆-C₁₄aryl)alkyl-O—; optionally substituted with from 1 to 3 C₁-C₆alkoxy;
(s) —C(O)NR₁₂R₁₂;
(t) NHC(O)R₁₃;
(u) —NHC(O)NR₁₂R₁₂;
(v) —NHC(O)OR₁₃;
(w) —NH(SO₂)—(C₁-C₆alkyl);
(x) or —(SO₂)—(C₁-C₆alkyl);
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl.

49. The compound of claim 48, wherein R₄ is hydrogen.

50. The compound of claim 48, wherein R₉ is C₁-C₆alkyl.

51. The compound of claim 48, wherein R₉ is (C₁-C₆alkoxy)carbonyl.

52. The compound of claim 48, wherein R₉ is C₁-C₈acyl.

53. The compound of claim 48, wherein R₉ is (C₆-C₁₄aryl)alkyl, wherein the ring portion of the (C₆-C₁₄aryl)alkyl group is optionally substituted by 1 to 3 substituents as specified in claim 48.

54. The compound of claim 48, wherein R₉ is heteroaryl(C₁-C₆alkyl), wherein the ring portion of the heteroaryl(C₁-C₆alkyl) group is optionally substituted by 1 to 3 substituents as specified in claim 48.

55. The compound of claim 48, wherein one or more X₃ is —N—.

56. The compound of claim 56, wherein one X₃ is —N—.

57. The compound of claim 48, wherein X₃ is —C(H)—.

58. The compound of claim 48, wherein X₃ is —C(C(O)H)—.

59. The compound of claim 48, wherein R₁₁ is hydrogen.

60. The compound of claim 48, wherein R₁₁ is hydroxyl.

61. The compound of claim 48, wherein R₁₁ is —NR₁₂R₁₂.

62. The compound of claim 48, wherein R₁₁ is —NHC(O)NR₁₂R₁₂.

63. The compound of claim 48, wherein R₁₁ is —NHC(O)OR₁₃.

64. The compound of claim 62, wherein R₁₂ is hydrogen.

65. The compound of claim 62, wherein R₁₂ is C₁-C₆alkyl.

66. The compound of claim 62, wherein R₁₂ is C₆-C₁₄aryl.

67. The compound of claim 64, wherein R₁₃ is C₁-C₆alkyl.

68. A compound of Formula IIb: 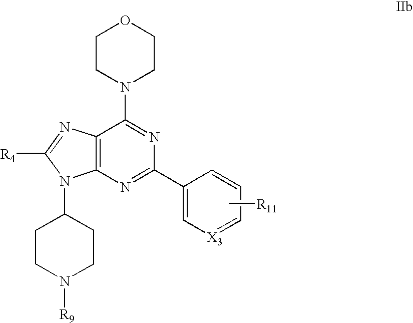
and pharmaceutically acceptable salts thereof, wherein
R₄ is hydrogen, C₁-C₆alkyl, C₂-C₁₀alkenyl, C₂-C₁₀alkynyl, or (C₆-C₁₄aryl)alkyl, wherein the C₁-C₆alkyl, C₂-C₁₀alkenyl, C₂-C₁₀alkynyl, and (C₆-C₁₄aryl)alkyl can be optionally substituted with hydroxyl, halogen, —NH₂, or —CN, provided that the substituent is not attached to a carbon of the C₂-C₁₀alkenyl double bond or the C₂-C₁₀alkynyl triple bond;
R₉ is:
(a) hydrogen;
(b) C₁-C₆alkyl;
(c) (C₁-C₆alkoxy)carbonyl;
(d) C₁-C₈acyl;
(e) (C₆-C₁₄aryl)alkyl, wherein the ring portion of the (C₆-C₁₄aryl)alkyl group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(f) heteroaryl(C₁-C₆alkyl), wherein the ring portion of the heteroaryl(C₁-C₆alkyl) group is optionally substituted by 1 to 3 substituents independently selected from:
(i) halogen,
(ii) C₁-C₆alkyl,
(iii) NH₂,
(iv) (C₁-C₆alkyl)amino,
(v) di(C₁-C₆alkyl)amino,
(vi) C₁-C₆alkoxy, wherein the C₁-C₆alkoxy is optionally substituted with NH₂, (C₁-C₆alkyl)amino, or di(C₁-C₆alkyl)amino,
(vii) C₁-C₉heterocycle,
(viii) C₆-C₁₄aryl,
(ix) and C₁-C₉heteroaryl;
(g) —C(O)OH;
(h) halogen;
(i) —NH₂;
(j) —NH(C₁-C₆alkyl);
(k) —N(C₁-C₆alkyl)(C₁-C₆alkyl);
(l) —N(C₁-C₃alkyl)C(O)(C₁-C₆alkyl);
(m) —NHC(O)(C₁-C₆alkyl);
(n) —NHC(O)H;
(o) —C(O)NH₂;
(p) —C(O)NH(C₁-C₆alkyl);
(q) —C(O)N(C₁-C₆alkyl)(C₁-C₆alkyl);
(r) —CN;
(s) —OH;
(t) —O(C₁-C₆alkyl);
(u) C₆-C₁₄aryl,
(v) C₁-C₉heteroaryl,
(w) or C₃-C₈carbocycle;
X₃ is —N—, or —C(H)—;
R₁₁ is:
(a) halogen,
(b) C₁-C₆alkyl,
(c) C₁-C₆alkoxy, optionally substituted with C₁-C₆alkoxy,
(d) C₁-C₆hydroxylalkyl,
(e) C₂-C₆alkenyl,
(f) C₂-C₆alkynyl,
(g) C₃-C₈carbocycle,
(h) C₆-C₁₄aryl,
(i) C₁-C₉heteroaryl,
(j) C₁-C₆perfluoroalkyl-,
(k) hydroxyl,
(l) NR₁₂R₁₂,
(m) NO₂,
(n) CN,
(o) CO₂H,
(p) CHO,
(q) C₆-C₁₄aryl-O—,
(r) (C₆-C₁₄aryl)alkyl-O—, optionally substituted with from 1 to 3 C₁-C₆alkoxy,
(s) —C(O)NR₁₂R₁₂,
(t) NHC(O)R₁₃,
(u) —NHC(O)NR₁₂R₁₂,
(v) —NHC(O)OR₁₃,
(w) —NH(SO₂)—(C₁-C₆alkyl),
(x) or —(SO₂)—(C₁-C₆alkyl);
each R₁₂ is each independently —H, —C₁-C₆alkyl, C₁-C₆alkoxy, C₆-C₁₄aryl, C₁-C₉heteroaryl, or —C₃-C₈carbocycle, or two R₁₂ radicals, when taken together with the nitrogen to which they are attached, can form a 3- to 7-membered nitrogen containing heterocycle wherein up to two of the carbon atoms of the heterocycle can be replaced by —N(R₈)—, —O—, —S—, —S(O)— or —S(O)₂—;
R₁₃ is independently —C₁-C₆alkyl, C₆-C₁₄aryl, C₁-C₉heteroaryl, or —C₃-C₈carbocycle;
R₈ is hydrogen, C₁-C₆alkyl, C₆-C₁₄aryl, or C₁-C₉heteroaryl.

69. The compound of claim 68, wherein R₄ is hydrogen.

70. The compound of claim 68, wherein R₉ is C₁-C₆alkyl.

71. The compound of claim 68, wherein R₉ is (C₁-C₆alkoxy)carbonyl.

72. The compound of claim 68, wherein R₉ is C₁-C₈acyl.

73. The compound of claim 68, wherein R₉ is (C₆-C₁₄aryl)alkyl, wherein the ring portion of the (C₆-C₁₄aryl)alkyl group is optionally substituted by 1 to 3 substituents as specified in claim 68.

74. The compound of claim 68, wherein R₉ is heteroaryl(C₁-C₆alkyl), wherein the ring portion of the heteroaryl(C₁-C₆alkyl) group is optionally substituted by 1 to 3 substituents as specified in claim 68.

75. The compound of claim 68, wherein X₃ is —N—.

76. The compound of claim 68, wherein X₃ is —C(H)—.

77. The compound of claim 68, wherein R₁₁ is hydrogen.

78. The compound of claim 68, wherein R₁₁ is hydroxyl.

79. The compound of claim 68, wherein R₁₁ is —NR₁₂R₁₂.

80. The compound of claim 68, wherein R₁₁ is —NHC(O)NR₁₂R₁₂.

81. The compound of claim 68, wherein R₁₁ is —NHC(O)OR₁₃.

82. The compound of claim 80, wherein one R₁₂ is hydrogen.

83. The compound of claim 80, wherein one R₁₂ is C₁-C₆alkyl.

84. The compound of claim 80, wherein one R₁₂ is C₆-C₁₄aryl.

85. The compound of claim 81, wherein R₁₃ is C₁-C₆alkyl.

86. A compound selected from the group consisting of:
6-morpholin-4-yl-2-(2-thienyl)-9H-purine;
6-morpholin-4-yl-2-(3-thienyl)-9H-purine;
2-(6-morpholin-4-yl-9H-purin-2-yl)phenol;
4-(6-morpholin-4-yl-9H-purin-2-yl)phenol;
[4-(6-morpholin-4-yl-9H-purin-2-yl)phenyl]methanol;
2-(1H-indol-5-yl)-6-morpholin-4-yl-9H-purine;
2-(1,3-benzodioxol-5-yl)-6-morpholin-4-yl-9H-purine;
6-morpholin-4-yl-2-(3-nitrophenyl)-9H-purine;
2-(1-benzothien-3-yl)-6-morpholin-4-yl-9H-purine;
6-morpholin-4-yl-2-[3-(trifluoromethyl)phenyl]-9H-purine;
2-(3-methylphenyl)-6-morpholin-4-yl-9H-purine;
2-(3-isopropylphenyl)-6-morpholin-4-yl-9H-purine;
3-(6-morpholin-4-yl-9H-purin-2-yl)benzonitrile;
2-biphenyl-3-yl-6-morpholin-4-yl-9H-purine;
N-(3-(6-morpholino-9H-purin-2-yl)phenyl)methanesulfonamide;
6-morpholin-4-yl-2-(2-phenoxyphenyl)-9H-purine;
N,N-dimethyl-4-(6-morpholin-4-yl-9H-purin-2-yl)benzamide;
2-(2,3-dihydro-1,4-benzodioxin-6-yl)-6-morpholin-4-yl-9H-purine;
2-(3-furyl)-6-morpholin-4-yl-9H-purine;
2-[3-(methylsulfonyl)phenyl]-6-morpholin-4-yl-9H-purine;
3-(6-Piperidin-1-yl-9H-purin-2-yl)-phenol;
2-(4-Methanesulfonyl-phenyl)-6-morpholin-4-yl-9H-purine;
2-[3-(3,5-Dimethoxy-benzyloxy)-phenyl]-6-morpholin-4-yl-9H-purine;
2-(4-Benzyloxy-3-chloro-phenyl)-6-morpholin-4-yl-9H-purine;
[3-(6-Morpholin-4-yl-9-piperidin-4-yl-9H-purin-2-yl)-phenyl]-methanol;
1-(4-(2-(3-(hydroxymethyl)phenyl)-6-morpholino-9H-purin-9-yl)piperidin-1-yl)ethanone;
3-(9-((1-benzylpiperidin-4-yl)methyl)-6-morpholino-9H-purin-2-yl)phenol;
3-(9-(1-benzylpiperidin-4-yl)-6-morpholino-9H-purin-2-yl)phenol;
(3-(9-((1-benzylpiperidin-4-yl)methyl)-6-morpholino-9H-purin-2-yl)phenyl)methanol;
5-(9-(1-benzylpiperidin-4-yl)-6-morpholino-9H-purin-2-yl)pyrimidin-2-amine;
5-[9-(1-benzylpiperidin-4-yl)-6-morpholin-4-yl-9H-purin-2-yl]pyridin-2-amine;
{3-[9-(1-benzylpiperidin-4-yl)-6-morpholin-4-yl-9H-purin-2-yl]phenyl}methanol;
5-[9-(1-benzylpiperidin-4-yl)-6-morpholin-4-yl-9H-purin-2-yl]nicotinaldehyde;
{5-[9-(1-benzylpiperidin-4-yl)-6-morpholin-4-yl-9H-purin-2-yl]pyridin-3-yl}methanol;
5-(6-morpholin-4-yl-9-piperidin-4-yl-9H-purin-2-yl)pyridin-3-ol;
5-(9-{1-[(5-methyl-2-thienyl)methyl]piperidin-4-yl}-6-morpholin-4-yl-9H-purin-2-yl)pyridin-3-ol;
5-{9-[1-(4-chlorobenzyl)piperidin-4-yl]-6-morpholin-4-yl-9H-purin-2-yl}pyridin-3-ol;
5-{6-morpholin-4-yl-9-[1-(1H-pyrrol-2-ylmethyl)piperidin-4-yl]-9H-purin-2-yl}pyridin-3-ol;
5-{9-[1-(4-methylbenzyl)piperidin-4-yl]-6-morpholin-4-yl-9H-purin-2-yl}pyridin-3-ol;
5-{9-[1-(6-fluoro-pyridin-3-ylmethyl)-piperidin-4-yl]-6-morpholin-4-yl-9H-purin-2-yl}-pyridin-3-ol;
2-(5-methoxypyridin-3-yl)-6-morpholin-4-yl-9H-purine;
5-(6-morpholin-4-yl-9H-purin-2-yl)pyridin-3-ol;
(3-{6-morpholin-4-yl-9-[1-(pyridin-2-ylmethyl)piperidin-4-yl]-9H-purin-2-yl}phenyl)methanol;
(3-{9-[1-(2-fluorobenzyl)piperidin-4-yl]-6-morpholin-4-yl-9H-purin-2-yl}phenyl)methanol;
(3-{9-[1-(4-fluorobenzyl)piperidin-4-yl]-6-morpholin-4-yl-9H-purin-2-yl}phenyl)methanol;
(3-{6-morpholin-4-yl-9-[1-(4-pyridin-4-ylbenzyl)piperidin-4-yl]-9H-purin-2-yl}phenyl)methanol;
{3-[9-(1-butylpiperidin-4-yl)-6-morpholin-4-yl-9H-purin-2-yl]phenyl}methanol;
(3-{9-[1-(2,4-difluorobenzyl)piperidin-4-yl]-6-morpholin-4-yl-9H-purin-2-yl}phenyl)methanol;
(3-{9-[1-(3-fluorobenzyl)piperidin-4-yl]-6-morpholin-4-yl-9H-purin-2-yl}phenyl)methanol;
{3-[9-(1-benzylpiperidin-4-yl)-6-morpholin-4-yl-9H-purin-2-yl]phenyl}methanol;
(3-{9-[1-(2-furylmethyl)piperidin-4-yl]-6-morpholin-4-yl-9H-purin-2-yl}phenyl)methanol;
4-(2-(3-methoxyphenyl)-9H-purin-6-yl)morpholine;
4-(2-phenyl-9H-purin-6-yl)morpholine];
3-(6-morpholino-9H-purin-2-yl)phenol;
tert-butyl 4-(2-choro-6-morpholino-9H-purin-9-yl)piperidine-1-carboxylate;
tert-butyl 4-{2-(3-hydroxyphenyl)-6-morpholino-9H-purin-9-yl}piperidine-1-carboxylate;
tert-butyl-4-{2-[5-(methoxymethoxy)pyridin-3-yl]-6-morpholin-4-yl-9H-purin-9-yl}piperidine-1-carboxylate;
tert-butyl 4-{2-[3-(hydroxymethyl)phenyl]-6-morpholin-4-yl-9H-purin-9-yl}piperidine-1-carboxylate;
(3-{6-morpholin-4-yl-9-[1-(2,4,6-trifluorobenzyl)piperidin-4-yl]-9H-purin-2-yl}phenyl)methanol;
[3-(9-{1-[(6-fluoropyridin-3-yl)methyl]piperidin-4-yl}-6-morpholin-4-yl-9H-purin-2-yl)phenyl]methanol;
(3-{9-[1-(3,4-difluorobenzyl)piperidin-4-yl]-6-morpholin-4-yl-9H-purin-2-yl}phenyl)methanol;
[3-(9-{1-[(6-chloropyridin-3-yl)methyl]piperidin-4-yl}-6-morpholin-4-yl-9H-purin-2-yl)phenyl]methanol;
[3-(9-{1-[(6-methoxypyridin-3-yl)methyl]piperidin-4-yl}-6-morpholin-4-yl-9H-purin-2-yl)phenyl]methanol;
[3-(9-{1-[(2,6-dimethoxypyridin-3-yl)methyl]piperidin-4-yl}-6-morpholin-4-yl-9H-purin-2-yl)phenyl]methanol;
[3-(9-{1-[(5-fluoropyridin-3-yl)methyl]piperidin-4-yl}-6-morpholin-4-yl-9H-purin-2-yl)phenyl]methanol;
[3-(9-{1-[(5-methyl-2-thienyl)methyl]piperidin-4-yl}-6-morpholin-4-yl-9H-purin-2-yl)phenyl]methanol;
(3-{6-morpholin-4-yl-9-[1-(1H-pyrrol-2-ylmethyl)piperidin-4-yl]-9H-purin-2-yl}phenyl)methanol;
(3-{9-[1-(2-chloro-4-fluorobenzyl)piperidin-4-yl]-6-morpholin-4-yl-9H-purin-2-yl}phenyl)methanol;
(3-{9-[1-(1H-imidazol-2-ylmethyl)piperidin-4-yl]-6-morpholin-4-yl-9H-purin-2-yl}phenyl)methanol;
[3-(9-{1-[(6-bromopyridin-2-yl)methyl]piperidin-4-yl}-6-morpholin-4-yl-9H-purin-2-yl)phenyl]methanol;
[3-(6-morpholin-4-yl-9-{1-[(6-morpholin-4-ylpyridin-2-yl)methyl]piperidin-4-yl}-9H-purin-2-yl)phenyl]methanol;
[3-(9-{1-[(5-fluoro-1H-indol-3-yl)methyl]piperidin-4-yl}-6-morpholin-4-yl-9H-purin-2-yl)phenyl]methanol;
(3-{9-[1-(5,6-dihydro-8H-imidazo[2,1-c][1,4]oxazin-3-ylmethyl)piperidin-4-yl]-6-morpholin-4-yl-9H-purin-2-yl}phenyl)methanol;
{3-[9-(1-{4-[3-dimethylamino)propoxy]benzyl}piperidin-4-yl)-6-morpholin-4-yl-9H-purin-2-yl]phenyl}methanol;
3-{6-morpholin-4-yl-9-[1-(pyridin-3-ylmethyl)piperidin-4-yl]-9H-purin-2-yl}phenol;
3-{6-morpholin-4-yl-9-[1-(pyridin-2-ylmethyl)piperidin-4-yl]-9H-purin-2-yl}phenol;
3-(9-{1-[(6-chloropyridin-3-yl)methyl]piperidin-4-yl}-6-morpholin-4-yl-9H-purin-2-yl)phenol;
3-(9-{1-[(6-methoxypyridin-3-yl)methyl]piperidin-4-yl}-6-morpholin-4-yl-9H-purin-2-yl)phenol;
3-(9-{1-[(2,6-dimethoxypyridin-4-yl)methyl]piperidin-4-yl}-6-morpholin-4-yl-9H-purin-2-yl)phenol;
3-(9-{1-[(6-bromopyridin-3-yl)methyl]piperidin-4-yl}-6-morpholin-4-yl-9H-purin-2-yl)phenol;
3-(6-morpholin-4-yl-9-{1-[(6-morpholin-4-ylpyridin-3-yl)methyl]piperidin-4-yl}-9H-purin-2-yl)phenol;
3-[9-(1-{[4-(dimethylamino)-1-naphthyl]methyl}piperidin-4-yl)-6-morpholin-4-yl-9H-purin-2-yl]phenol;
3-(9-{1-[(6-fluoropyridin-3-yl)methyl]piperidin-4-yl}-6-morpholin-4-yl-9H-purin-2-yl)phenol;
3-[6-morpholin-4-yl-9-(2-piperidin-1-ylethyl)-9H-purin-2-yl]phenol;
{3-[6-morpholin-4-yl-9-(2-piperidin-1-ylethyl)-9H-purin-2-yl]phenyl}methanol;
5-[9-(1-benzylpiperidin-4-yl)-6-morpholin-4-yl-9H-purin-2-yl]pyridin-3-ol;
5-(9-{1-[(6-fluoropyridin-3-yl)methyl]piperidin-4-yl}-6-morpholin-4-yl-9H-purin-2-yl)pyridin-3-ol;
1-methyl-3-(4-(6-morpholino-9-(1-(pyridin-3-ylmethyl)piperidin-4-yl)-9H-purin-2-yl)phenyl)urea;
1-ethyl-3-(4-(6-morpholino-9-(1-(pyridin-3-ylmethyl)piperidin-4-yl)-9H-purin-2-yl)phenyl)urea;
and 1-(2-(3-hydroxyphenyl)-6-morpholino-9H-purin-9-yl)prop-2-en-1-one.

87. A composition comprising the compound claim 1 and a pharmaceutically acceptable carrier.

88. The composition of claim 87, wherein the pharmaceutically acceptable carrier is suitable for oral administration and the composition comprises an oral dosage form.

89. A composition comprising the compound claim 18 and a pharmaceutically acceptable carrier.

90. The composition of claim 89, wherein the pharmaceutically acceptable carrier is suitable for oral administration and the composition comprises an oral dosage form.

91. A composition comprising the compound claim 35 and a pharmaceutically acceptable carrier.

92. The composition of claim 91, wherein the pharmaceutically acceptable carrier is suitable for oral administration and the composition comprises an oral dosage form.

93. A composition comprising the compound claim 48 and a pharmaceutically acceptable carrier.
The composition of claim 93, wherein the pharmaceutically acceptable carrier is suitable for oral administration and the composition comprises an oral dosage form.

94. A composition comprising the compound claim 68 and a pharmaceutically acceptable carrier.

95. The composition of claim 94, wherein the pharmaceutically acceptable carrier is suitable for oral administration and the composition comprises an oral dosage form.

96. A composition comprising the compound claim 1; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

97. The composition of claim 96, wherein the second compound is Avastin.

98. A composition comprising the compound claim 18; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

99. The composition of claim 98, wherein the second compound is Avastin.

100. A composition comprising the compound claim 35; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

101. The composition of claim 101, wherein the second compound is Avastin.

102. A composition comprising the compound claim 48; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

103. The composition of claim 102, wherein the second compound is Avastin.

104. A composition comprising the compound claim 68; a second compound selected from the group consisting of a topoisomerase I inhibitor, procarbazine, dacarbazine, gemcitabine, capecitabine, methotrexate, taxol, taxotere, mercaptopurine, thioguanine, hydroxyurea, cytarabine, cyclophosphamide, ifosfamide, nitrosoureas, cisplatin, carboplatin, mitomycin, dacarbazine, procarbizine, etoposide, teniposide, campathecins, bleomycin, doxorubicin, idarubicin, daunorubicin, dactinomycin, plicamycin, mitoxantrone, L-asparaginase, doxorubicin, epirubicin, 5-fluorouracil, docetaxel, paclitaxel, leucovorin, levamisole, irinotecan, estramustine, etoposide, nitrogen mustards, BCNU, carmustine, lomustine, vinblastine, vincristine, vinorelbine, cisplatin, carboplatin, oxaliplatin, imatinib mesylate, Avastin (bevacizumab), hexamethylmelamine, topotecan, tyrosine kinase inhibitors, tyrphostins, herbimycin A, genistein, erbstatin, and lavendustin A; and a pharmaceutically acceptable carrier.

105. The composition of claim 104, wherein the second compound is Avastin.

106. A method of treating a PI3K-related disorder, comprising administering to a mammal in need thereof the compound claim 1 in an amount effective to treat a PI3K-related disorder.

107. The method of claim 106, wherein the PI3K-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

108. The method of claim 107, wherein the PI3K-related disorder is cancer.

109. The method of claim 108, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

110. A method of treating an mTOR-related disorder, comprising administering to a mammal in need thereof the compound claim 1 in an amount effective to treat an mTOR-related disorder.

111. The method of claim 110, wherein the mTOR-related disorder is selected from restenosis, atherosclerosis, bone disorders, arthritis, diabetic retinopathy, psoriasis, benign prostatic hypertrophy, atherosclerosis, inflammation, angiogenesis, immunological disorders, pancreatitis, kidney disease, and cancer.

112. The method of claim 111, wherein the mTOR-related disorder is cancer.

113. The method of claim 112, wherein the cancer is selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer.

114. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 96 in an amount effective to treat the cancer.

115. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 98 in an amount effective to treat the cancer.

116. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 100 in an amount effective to treat the cancer.

117. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 102 in an amount effective to treat the cancer.

118. A method of treating a cancer selected from the group consisting of leukemia, skin cancer, bladder cancer, breast cancer, uterus cancer, ovary cancer, prostate cancer, lung cancer, colon cancer, pancreas cancer, renal cancer, gastric cancer, and brain cancer comprising administering to a mammal in need thereof the composition of claim 104 in an amount effective to treat the cancer.

119. A method of inhibiting mTOR in a subject, comprising administering to a subject in need thereof the compound claims 1 in an amount effective to inhibit mTOR.

120. A method of inhibiting PI3K in a subject, comprising administering to a subject in need thereof the compound claim 1 in an amount effective to inhibit PI3K.

121. A method of synthesizing a compound of claim 1 comprising reacting a compound of the formula F′: 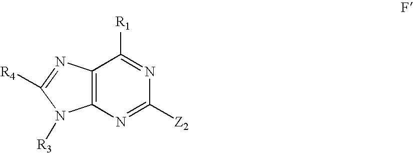
wherein Z₂ is halogen and R₁—R₄ are as defined above in claim 1 with a boronic acid of the formula R₂B(OH)₂ thereby providing a compound having the formula Ib: 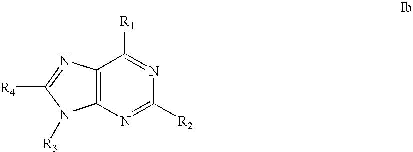
pharmaceutically acceptable salts thereof.

122. The method of claim 121 further comprising:
a) reacting the compound of Formula B′ with an amine of the formula R₁—H: 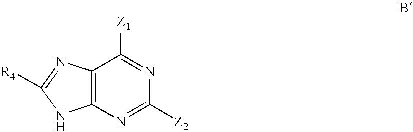
wherein Z₁ is halogen thereby providing a compound of the formula C′: 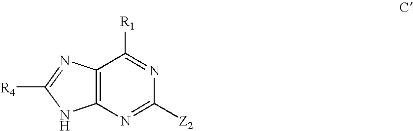
b) reacting a compound of the formula C′ with an alcohol R₃OH thereby providing a compound of formula F′.
